(19) United States
(12) Patent Application Publication
(10) Pub. No.: US 2025/0025427 A1
(43) Pub. Date: Jan. 23, 2025

(54) MIXED AMIDE ESTER CONTAINING LIPIDS FOR USE IN LIPID NANOPARTICLES

(71) Applicant: Acuitas Therapeutics, Inc., Vancouver (CA)

(72) Inventors: Xinyao Du, Richmond (CA); Polina Blagojevic, Vancouver (CA); Stephen Paul Arns, Vancouver (CA)

(21) Appl. No.: 18/744,413

(22) Filed: Jun. 14, 2024

Related U.S. Application Data
(60) Provisional application No. 63/508,778, filed on Jun. 16, 2023.

(51) Int. Cl. A61K 9/51; C07C 235/74; C07C 237/12; C07D 233/61; C07D 295/13

(52) U.S. Cl. CPC A61K 9/5123; C07C 235/74; C07C 237/12; C07D 233/61; C07D 295/13; C07C 2601/14

(57) ABSTRACT

Compounds are provided having the following Formula (I):

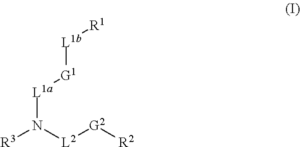

or a pharmaceutically acceptable salt, tautomer, or stereoisomer thereof, wherein G¹, G², R¹, R², R³, L^{1a}, L^{1b}, and L² are as defined herein. Use of the compounds as a component of lipid nanoparticle formulations for delivery of a therapeutic agent, compositions comprising the compounds and methods for their use and preparation are also provided.

### BACKGROUND

### Technical Field

The present disclosure generally relates to novel cationic lipids that can be used in combination with other lipid components, such as neutral lipids, cholesterol, and polymer conjugated lipids, to form lipid nanoparticles with oligonucleotides, to facilitate the intracellular delivery of therapeutic nucleic acids (e.g., oligonucleotides, messenger RNA) both in vitro and in vivo.

### Description of the Related Art

There are many challenges associated with the delivery of nucleic acids to affect a desired response in a biological system. Nucleic acid-based therapeutics have enormous potential but there remains a need for more effective delivery of nucleic acids to appropriate sites within a cell or organism to realize this potential. Therapeutic nucleic acids include, e.g., messenger RNA (mRNA), antisense oligonucleotides, ribozymes, DNAzymes, plasmids, immune stimulating nucleic acids, antagomir, antimir, mimic, supermir, and aptamers. Some nucleic acids, such as mRNA or plasmids, can be used to effect expression of specific cellular products as would be useful in the treatment of, for example, diseases related to a deficiency of a protein or enzyme. The therapeutic applications of translatable nucleotide delivery are extremely broad as constructs can be synthesized to produce any chosen protein sequence, whether indigenous to the system. The expression products of the nucleic acid can augment existing levels of protein, replace missing or non-functional versions of a protein, or introduce a new protein and associated functionality in a cell or organism.

Some nucleic acids, such as miRNA inhibitors, can be used to effect expression of specific cellular products that are regulated by miRNA as would be useful in the treatment of, for example, diseases related to deficiency of protein or enzyme. The therapeutic applications of miRNA inhibition are extremely broad as constructs can be synthesized to inhibit one or more miRNA that would in turn regulate the expression of mRNA products. The inhibition of endogenous miRNA can augment its downstream target endogenous protein expression and restore proper function in a cell or organism as a means to treat disease associated to a specific miRNA or a group of miRNA. Other nucleic acids can down-regulate intracellular levels of specific mRNA and, as a result, down-regulate the synthesis of the corresponding proteins through processes such as RNA interference (RNAi) or complementary binding of antisense RNA. The therapeutic applications of antisense oligonucleotide and RNAi are also extremely broad, since oligonucleotide constructs can be synthesized with any nucleotide sequence directed against a target mRNA. Targets may include mRNAs from normal cells, mRNAs associated with disease-states, such as cancer, and mRNAs of infectious agents, such as viruses. To date, antisense oligonucleotide constructs have shown the ability to specifically down-regulate target proteins through degradation of the cognate mRNA in both in vitro and in vivo models. In addition, antisense oligonucleotide constructs are currently being evaluated in clinical studies.

However, two problems currently face using oligonucleotides in therapeutic contexts. First, free RNAs are susceptible to nuclease digestion in plasma. Second, free RNAs have limited ability to gain access to the intracellular compartment where the relevant translation machinery resides. Lipid nanoparticles formed from cationic lipids with other lipid components, such as neutral lipids, cholesterol, PEG, PEGylated lipids, and oligonucleotides have been used to block degradation of the RNAs in plasma and facilitate the cellular uptake of the oligonucleotides.

There remains a need for improved cationic lipids and lipid nanoparticles for the delivery of oligonucleotides. Preferably, these lipid nanoparticles would provide optimal drug to lipid ratios, protect the nucleic acid from degradation and clearance in serum, be suitable for systemic delivery, and provide intracellular delivery of the nucleic acid. In addition, these lipid-nucleic acid particles should be well-tolerated and provide an adequate therapeutic index, such that patient treatment at an effective dose of the nucleic acid is not associated with unacceptable toxicity and/or risk to the patient. The present disclosure provides these and related advantages.

### BRIEF SUMMARY

In brief, the present disclosure provides lipid compounds, including stereoisomers, pharmaceutically acceptable salts, or tautomers thereof, which can be used alone or in combination with other lipid components such as neutral lipids, charged lipids, steroids (including for example, all sterols) and/or their analogs, and/or polymer conjugated lipids to form lipid nanoparticles for the delivery of therapeutic agents. In some instances, the lipid nanoparticles are used to deliver nucleic acids such as antisense and/or messenger RNA. Methods for use of such lipid nanoparticles for treatment of various diseases or conditions, such as those caused by infectious entities and/or insufficiency of a protein, are also provided.

In one embodiment, compounds having the following structure of Formula (I) are provided:

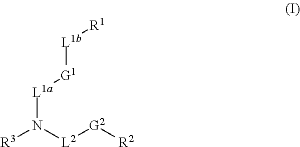

or a pharmaceutically acceptable salt, tautomer, or stereoisomer thereof, wherein G¹, G², R¹, R², R³, L^{1a}, L^{1b}, and L² are as defined herein.

Pharmaceutical compositions comprising one or more of the foregoing compounds of Formula (I) and a therapeutic agent are also provided. In some embodiments, the pharmaceutical compositions further comprise one or more components selected from neutral lipids, charged lipids, steroids, and polymer conjugated lipids. Such compositions are useful for formation of lipid nanoparticles for the delivery of the therapeutic agent.

In other embodiments, the present disclosure provides a method for administering a therapeutic agent to a patient in need thereof, the method comprising preparing a composition of lipid nanoparticles comprising the compound of Formula (I) and a therapeutic agent and delivering the composition to the patient. Such methods are useful for inducing expression of a protein in a subject, for example for expressing an antigen for purposes of vaccination or a gene editing protein.

These and other aspects of the disclosure will be apparent upon reference to the following detailed description.

### DETAILED DESCRIPTION

In the following description, certain specific details are set forth to provide a thorough understanding of various embodiments of the disclosure. However, one skilled in the art will understand that the embodiments of the disclosure may be practiced without these details.

The present disclosure is based, in part, upon the discovery of novel cationic lipids that provide advantages when used in lipid nanoparticles for the in vivo delivery of an active or therapeutic agent such as a nucleic acid into a cell of a mammal. Embodiments of the present disclosure provide nucleic acid-lipid nanoparticle compositions comprising one or more of the novel cationic lipids described herein that provide increased activity of the nucleic acid and improved tolerability of the compositions in vivo, resulting in a significant increase in the therapeutic index as compared to nucleic acid-lipid nanoparticle compositions previously described.

In certain embodiments, the present disclosure provides novel cationic lipids that enable the formulation of improved compositions for the in vitro and in vivo delivery of mRNA and/or other oligonucleotides. In some embodiments, these improved lipid nanoparticle compositions are useful for expression of protein encoded by mRNA. In other embodiments, these improved lipid nanoparticles compositions are useful for upregulation of endogenous protein expression by delivering miRNA inhibitors targeting one specific miRNA or a group of miRNA regulating one target mRNA or several mRNA. In other embodiments, these improved lipid nanoparticle compositions are useful for down-regulating (e.g., silencing) the protein levels and/or mRNA levels of target genes. In some other embodiments, the lipid nanoparticles are also useful for delivery of mRNA and plasmids for expression of transgenes. In yet other embodiments, the lipid nanoparticle compositions are useful for inducing a pharmacological effect resulting from expression of a protein, e.g., increased production of red blood cells through the delivery of a suitable erythropoietin mRNA, or protection against infection through delivery of mRNA encoding for a suitable antibody.

The lipid nanoparticles and compositions of the present disclosure may be used for a variety of purposes, including the delivery of encapsulated or associated (e.g., complexed) therapeutic agents such as nucleic acids to cells, both in vitro and in vivo. Accordingly, embodiments of the present disclosure provide methods of treating or preventing diseases or disorders in a subject in need thereof by contacting the subject with a lipid nanoparticle that encapsulates or is associated with a suitable therapeutic agent, wherein the lipid nanoparticle comprises one or more of the novel cationic lipids described herein.

As described herein, embodiments of the lipid nanoparticles of the present disclosure are particularly useful for the delivery of nucleic acids, including, e.g., mRNA, antisense oligonucleotide, plasmid DNA, microRNA (miRNA), miRNA inhibitors (antagomirs/antimirs), messenger-RNA-interfering complementary RNA (micRNA), DNA, multivalent RNA, dicer substrate RNA, complementary DNA (cDNA), etc. Therefore, the lipid nanoparticles and compositions of the present disclosure may be used to induce expression of a desired protein both in vitro and in vivo by contacting cells with a lipid nanoparticle comprising one or more novel cationic lipids described herein, wherein the lipid nanoparticle encapsulates or is associated with a nucleic acid that is expressed to produce the desired protein (e.g., a messenger RNA or plasmid encoding the desired protein). Alternatively, the lipid nanoparticles and compositions of the present disclosure may be used to decrease the expression of target genes and proteins both in vitro and in vivo by contacting cells with a lipid nanoparticle comprising one or more novel cationic lipids described herein, wherein the lipid nanoparticle encapsulates or is associated with a nucleic acid that reduces target gene expression (e.g., an antisense oligonucleotide or small interfering RNA (siRNA)). The lipid nanoparticles and compositions of the present disclosure may also be used for co-delivery of different nucleic acids (e.g., mRNA and plasmid DNA) separately or in combination, such as may be useful to provide an effect requiring co-localization of different nucleic acids (e.g., mRNA encoding for a suitable gene modifying enzyme and DNA segment(s) for incorporation into the host genome).

Nucleic acids for use with this disclosure may be prepared according to any available technique. For mRNA, the primary methodology of preparation is, but not limited to, enzymatic synthesis (also termed in vitro transcription) which currently represents the most efficient method to produce long sequence-specific mRNA. In vitro transcription describes a process of template-directed synthesis of RNA molecules from an engineered DNA template comprised of an upstream bacteriophage promoter sequence (e.g., including but not limited to that from the T7, T3, and SP6 coliphage) linked to a downstream sequence encoding the gene of interest. Template DNA can be prepared for in vitro transcription from a number of sources with appropriate techniques which are well known in the art including, but not limited to, plasmid DNA and polymerase chain reaction amplification (see Linpinsel, J. L and Conn, G. L., General protocols for preparation of plasmid DNA template and Bowman, J. C., Azizi, B., Lenz, T. K., Ray, P., and Williams, L. D. in RNA in vitro transcription and RNA purification by denaturing PAGE in Recombinant and in vitro RNA syntheses Methods v. 941 Conn G. L. (ed), New York, N. Y. Humana Press, 2012).

Transcription of the RNA occurs in vitro using the linearized DNA template in the presence of the corresponding RNA polymerase and adenosine, guanosine, uridine, and cytidine ribonucleoside triphosphates (rNTPs) under conditions that support polymerase activity while minimizing potential degradation of the resultant mRNA transcripts. In vitro transcription can be performed using a variety of commercially available kits including, but not limited to RiboMax Large Scale RNA Production System (Promega), MegaScript Transcription kits (Life Technologies) as well as with commercially available reagents including RNA polymerases and rNTPs. The methodology for in vitro transcription of mRNA is well known in the art. (see, e.g., Losick, R., 1972, In vitro transcription, Ann Rev Biochem v. 41 409-46; Kamakaka, R. T. and Kraus, W. L. 2001. In Vitro Transcription. Current Protocols in Cell Biology. 2:11.6:11.6.1-11.6.17; Beckert, B. And Masquida, B., (2010) Synthesis of RNA by In Vitro Transcription in RNA in Methods in Molecular Biology v. 703 (Neilson, H. Ed), New York, N.Y. Humana Press, 2010; Brunelle, J. L. and Green, R., 2013, Chapter Five—In vitro transcription from plasmid or PCR-amplified DNA, Methods in Enzymology v. 530, 101-114; all of which are incorporated herein by reference).

The desired in vitro transcribed mRNA is then purified from the undesired components of the transcription or associated reactions (including unincorporated rNTPs, protein enzyme, salts, short RNA oligos etc.). Techniques for the isolation of the mRNA transcripts are well known in the art. Well known procedures include phenol/chloroform extraction or precipitation with either alcohol (ethanol, isopropanol) in the presence of monovalent cations or lithium chloride. Additional, non-limiting examples of purification procedures which can be used include size exclusion chromatography (Lukavsky, P. J. and Puglisi, J. D., 2004, Large-scale preparation and purification of polyacrylamide-free RNA oligonucleotides, RNA v. 10, 889-893), silica-based affinity chromatography and polyacrylamide gel electrophoresis (Bowman, J. C., Azizi, B., Lenz, T. K., Ray, P., and Williams, L. D. in RNA in vitro transcription and RNA purification by denaturing PAGE in Recombinant and in vitro RNA syntheses Methods v. 941 Conn G. L. (ed), New York, N. Y. Humana Press, 2012). Purification can be performed using a variety of commercially available kits including, but not limited to SV Total Isolation System (Promega) and In Vitro Transcription Cleanup and Concentration Kit (Norgen Biotek).

Furthermore, while reverse transcription can yield large quantities of mRNA, the products can contain several aberrant RNA impurities associated with undesired polymerase activity which may need to be removed from the full-length mRNA preparation. These include short RNAs that result from abortive transcription initiation as well as double-stranded RNA (dsRNA) generated by RNA-dependent RNA polymerase activity, RNA-primed transcription from RNA templates and self-complementary 3′ extension. It has been demonstrated that these contaminants with dsRNA structures can lead to undesired immunostimulatory activity through interaction with various innate immune sensors in eukaryotic cells that function to recognize specific nucleic acid structures and induce potent immune responses. This in turn, can dramatically reduce mRNA translation since protein synthesis is reduced during the innate cellular immune response. Therefore, additional techniques to remove these dsRNA contaminants have been developed and are known in the art including but not limited to scalable HPLC purification (see, e.g., Kariko, K., Muramatsu, H., Ludwig, J. And Weissman, D., 2011, Generating the optimal mRNA for therapy: HPLC purification eliminates immune activation and improves translation of nucleoside-modified, protein-encoding mRNA, Nucl Acid Res, v. 39 e142; Weissman, D., Pardi, N., Muramatsu, H., and Kariko, K., HPLC Purification of in vitro transcribed long RNA in Synthetic Messenger RNA and Cell Metabolism Modulation in Methods in Molecular Biology v. 969 (Rabinovich, P. H. Ed), 2013). HPLC purified mRNA has been reported to be translated at much greater levels, particularly in primary cells and in vivo.

A significant variety of modifications have been described in the art which are used to alter specific properties of in vitro transcribed mRNA and improve its utility. These include but are not limited to modifications to the 5′ and 3′ termini of the mRNA. Endogenous eukaryotic mRNA typically contains a cap structure on the 5′-end of a mature molecule which plays an important role in mediating binding of the mRNA Cap Binding Protein (CBP), which is in turn responsible for enhancing mRNA stability in the cell and efficiency of mRNA translation. Therefore, highest levels of protein expression are achieved with capped mRNA transcripts. The 5′-cap contains a 5′-5′-triphosphate linkage between the 5′-most nucleotide and guanine nucleotide. The conjugated guanine nucleotide is methylated at the N7 position. Additional modifications include methylation of the ultimate and penultimate most 5′-nucleotides on the 2′-hydroxyl group.

Multiple distinct cap structures can be used to generate the 5′-cap of in vitro transcribed synthetic mRNA. 5′-capping of synthetic mRNA can be performed co-transcriptionally with chemical cap analogs (i.e., capping during in vitro transcription). For example, the Anti-Reverse Cap Analog (ARCA) cap contains a 5′-5′-triphosphate guanine-guanine linkage where one guanine contains an N7 methyl group as well as a 3′-O-methyl group. However, up to 20% of transcripts remain uncapped during this co-transcriptional process and the synthetic cap analog is not identical to the 5′-cap structure of an authentic cellular mRNA, potentially reducing translatability and cellular stability. Alternatively, synthetic mRNA molecules may also be enzymatically capped post-transcriptionally. These may generate a more authentic 5′-cap structure that more closely mimics, either structurally or functionally, the endogenous 5′-cap which have enhanced binding of cap binding proteins, increased half-life, reduced susceptibility to 5′ endonucleases and/or reduced 5′ decapping. Numerous synthetic 5′-cap analogs have been developed and are known in the art to enhance mRNA stability and translatability (see, e.g., Grudzien-Nogalska, E., Kowalska, J., Su, W., Kuhn, A. N., Slepenkov, S. V., Darynkiewicz, E., Sahin, U., Jemielity, J., and Rhoads, R. E., Synthetic mRNAs with superior translation and stability properties in Synthetic Messenger RNA and Cell Metabolism Modulation in Methods in Molecular Biology v. 969 (Rabinovich, P. H. Ed), 2013).

On the 3′-terminus, a long chain of adenine nucleotides (poly-A tail) is normally added to mRNA molecules during RNA processing. Immediately after transcription, the 3′ end of the transcript is cleaved to free a 3′ hydroxyl to which poly-A polymerase adds a chain of adenine nucleotides to the RNA in a process called polyadenylation. The poly-A tail has been extensively shown to enhance both translational efficiency and stability of mRNA (see Bernstein, P. and Ross, J., 1989, Poly (A), poly (A) binding protein and the regulation of mRNA stability, Trends Bio Sci v. 14 373-377; Guhaniyogi, J. And Brewer, G., 2001, Regulation of mRNA stability in mammalian cells, Gene, v. 265, 11-23; Dreyfus, M. And Regnier, P., 2002, The poly (A) tail of mRNAs: Bodyguard in eukaryotes, scavenger in bacteria, Cell, v. 111, 611-613).

Poly (A) tailing of in vitro transcribed mRNA can be achieved using various approaches including, but not limited to, cloning of a poly (T) tract into the DNA template or by post-transcriptional addition using Poly (A) polymerase. The first case allows in vitro transcription of mRNA with poly (A) tails of defined length, depending on the size of the poly (T) tract, but requires additional manipulation of the template. The latter case involves the enzymatic addition of a poly (A) tail to in vitro transcribed mRNA using poly (A) polymerase which catalyzes the incorporation of adenine residues onto the 3′termini of RNA, requiring no additional manipulation of the DNA template, but results in mRNA with poly(A) tails of heterogenous length. 5′-capping and 3′-poly (A) tailing can be performed using a variety of commercially available kits including, but not limited to Poly (A) Polymerase Tailing kit (EpiCenter), mMESSAGE mMACHINE T7 Ultra kit and Poly (A) Tailing kit (Life Technologies) as well as with commercially available reagents, various ARCA caps, Poly (A) polymerase, etc.

In addition to 5′ cap and 3′ poly adenylation, other modifications of the in vitro transcripts have been reported to provide benefits as related to efficiency of translation and stability. It is well known in the art that pathogenic DNA and RNA can be recognized by a variety of sensors within eukaryotes and trigger potent innate immune responses. The ability to discriminate between pathogenic and self-DNA and RNA has been shown to be based, at least in part, on structure and nucleoside modifications since most nucleic acids from natural sources contain modified nucleosides. In contrast, in vitro synthesized RNA lacks these modifications, thus rendering it immunostimulatory which in turn can inhibit effective mRNA translation as outlined above. The introduction of modified nucleosides into in vitro transcribed mRNA can be used to prevent recognition and activation of RNA sensors, thus mitigating this undesired immunostimulatory activity and enhancing translation capacity (see, e.g., Kariko, K. And Weissman, D. 2007, Naturally occurring nucleoside modifications suppress the immunostimulatory activity of RNA: implication for therapeutic RNA development, Curr Opin Drug Discov Devel, v. 10 523-532; Pardi, N., Muramatsu, H., Weissman, D., Kariko, K., In vitro transcription of long RNA containing modified nucleosides in Synthetic Messenger RNA and Cell Metabolism Modulation in Methods in Molecular Biology v. 969 (Rabinovich, P. H. Ed), 2013); Kariko, K., Muramatsu, H., Welsh, F. A., Ludwig, J., Kato, H., Akira, S., Weissman, D., 2008, Incorporation of Pseudouridine Into mRNA Yields Superior Nonimmunogenic Vector With Increased Translational Capacity and Biological Stability, Mol Ther v. 16, 1833-1840. The modified nucleosides and nucleotides used in the synthesis of modified RNAs can be prepared monitored and utilized using general methods and procedures known in the art. A large variety of nucleoside modifications are available that may be incorporated alone or in combination with other modified nucleosides to some extent into the in vitro transcribed mRNA (see, e.g., US Publication No. 2012/0251618). In vitro synthesis of nucleoside-modified mRNA has been reported to have reduced ability to activate immune sensors with a concomitant enhanced translational capacity.

Other components of mRNA which can be modified to provide benefit in terms of translatability and stability include the 5′ and 3′ untranslated regions (UTR). Optimization of the UTRs (favorable 5′ and 3′ UTRs can be obtained from cellular or viral RNAs), either both or independently, have been shown to increase mRNA stability and translational efficiency of in vitro transcribed mRNA (see, e.g., Pardi, N., Muramatsu, H., Weissman, D., Kariko, K., In vitro transcription of long RNA containing modified nucleosides in Synthetic Messenger RNA and Cell Metabolism Modulation in Methods in Molecular Biology v. 969 (Rabinovich, P. H. Ed), 2013).

In addition to mRNA, other nucleic acid payloads may be used for this disclosure. For oligonucleotides, methods of preparation include but are not limited to chemical synthesis and enzymatic, chemical cleavage of a longer precursor, in vitro transcription as described above, etc. Methods of synthesizing DNA and RNA nucleotides are widely used and well known in the art (see, e.g., Gait, M. J. (ed.) Oligonucleotide synthesis: a practical approach, Oxford [Oxfordshire], Washington, D.C.: IRL Press, 1984; and Herdewijn, P. (ed.) Oligonucleotide synthesis: methods and applications, Methods in Molecular Biology, v. 288 (Clifton, N.J.) Totowa, N.J.: Humana Press, 2005; both of which are incorporated herein by reference).

For plasmid DNA, preparation for use with this disclosure commonly utilizes but is not limited to expansion and isolation of the plasmid DNA in vitro in a liquid culture of bacteria containing the plasmid of interest. The presence of a gene in the plasmid of interest that encodes resistance to a particular antibiotic (penicillin, kanamycin, etc.) allows those bacteria containing the plasmid of interest to selective grow in antibiotic-containing cultures. Methods of isolating plasmid DNA are widely used and well known in the art (see, e.g., Heilig, J., Elbing, K. L. and Brent, R (2001) Large-Scale Preparation of Plasmid DNA. Current Protocols in Molecular Biology. 41:II:1.7:1.7.1-1.7.16; Rozkov, A., Larsson, B., Gillström, S., BjOrnestedt, R. and Schmidt, S. R. (2008), Large-scale production of endotoxin-free plasmids for transient expression in mammalian cell culture. Biotechnol. Bioeng., 99: 557-566; and U.S. Pat. No. 6,197,553B1). Plasmid isolation can be performed using a variety of commercially available kits including, but not limited to Plasmid Plus (Qiagen), GenJET plasmid MaxiPrep (Thermo) and PureYield MaxiPrep (Promega) kits as well as with commercially available reagents.

Various exemplary embodiments of the cationic lipids of the present disclosure, lipid nanoparticles and compositions comprising the same, and their use to deliver active or therapeutic agents such as nucleic acids to modulate gene and protein expression, are described in further detail below.

As used herein, the following terms have the meanings ascribed to them unless specified otherwise.

Unless the context requires otherwise, throughout the present specification and claims, the word “comprise” and variations thereof, such as, “comprises” and “comprising” are to be construed in an open and inclusive sense, that is, as “including, but not limited to”.

Reference throughout this specification to “one embodiment” or “an embodiment” means that a particular feature, structure, or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the features, structures, or characteristics may be combined in any suitable manner in one or more embodiments.

Unless defined otherwise, all technical and scientific terms used herein have the same meaning as is commonly understood by one of skill in the art to which this disclosure belongs. As used in the specification and claims, the singular form “a”, “an” and “the” include plural references unless the context clearly dictates otherwise.

The phrase “induce expression of a desired protein” refers to the ability of a nucleic acid to increase expression of the desired protein. To examine the extent of protein expression, a test sample (e.g., a sample of cells in culture expressing the desired protein) or a test mammal (e.g., a mammal such as a human or an animal model such as a rodent (e.g., mouse) or a non-human primate (e.g., monkey) model) is contacted with a nucleic acid (e.g., nucleic acid in combination with a lipid of the present disclosure). Expression of the desired protein in the test sample or test animal is compared to expression of the desired protein in a control sample (e.g., a sample of cells in culture expressing the desired protein) or a control mammal (e.g., a mammal such as a human or an animal model such as a rodent (e.g., mouse) or non-human primate (e.g., monkey) model) that is not contacted with or administered the nucleic acid. When the desired protein is present in a control sample or a control mammal, the expression of a desired protein in a control sample or a control mammal may be assigned a value of 1.0. In some embodiments, inducing expression of a desired protein is achieved when the ratio of desired protein expression in the test sample or the test mammal to the level of desired protein expression in the control sample or the control mammal is greater than 1, for example, about 1.1, 1.5, 2.0. 5.0 or 10.0. When a desired protein is not present in a control sample or a control mammal, inducing expression of a desired protein is achieved when any measurable level of the desired protein in the test sample or the test mammal is detected. One of ordinary skill in the art will understand appropriate assays to determine the level of protein expression in a sample, for example dot blots, northern blots, in situ hybridization, ELISA, immunoprecipitation, enzyme function, and phenotypic assays, or assays based on reporter proteins that can produce fluorescence or luminescence under appropriate conditions.

The phrase “inhibiting expression of a target gene” refers to the ability of a nucleic acid to silence, reduce, or inhibit the expression of a target gene. To examine the extent of gene silencing, a test sample (e.g., a sample of cells in culture expressing the target gene) or a test mammal (e.g., a mammal such as a human or an animal model such as a rodent (e.g., mouse) or a non-human primate (e.g., monkey) model) is contacted with a nucleic acid that silences, reduces, or inhibits expression of the target gene. Expression of the target gene in the test sample or test animal is compared to expression of the target gene in a control sample (e.g., a sample of cells in culture expressing the target gene) or a control mammal (e.g., a mammal such as a human or an animal model such as a rodent (e.g., mouse) or non-human primate (e.g., monkey) model) that is not contacted with or administered the nucleic acid. The expression of the target gene in a control sample or a control mammal may be assigned a value of 100%. In particular embodiments, silencing, inhibition, or reduction of expression of a target gene is achieved when the level of target gene expression in the test sample or the test mammal relative to the level of target gene expression in the control sample or the control mammal is about 95%, 90%, 85%, 80%, 75%, 70%, 65%, 60%, 55%, 50%, 45%, 40%, 35%, 30%, 25%, 20%, 15%, 10%, 5%, or 0%. In other words, the nucleic acids are capable of silencing, reducing, or inhibiting the expression of a target gene by at least about 5%, 10%, 15%, 20%, 25%, 30%, 35%, 40%, 45%, 50%, 55%, 60%, 65%, 70%, 75%, 80%, 85%, 90%, 95%, or 100% in a test sample or a test mammal relative to the level of target gene expression in a control sample or a control mammal not contacted with or administered the nucleic acid. Suitable assays for determining the level of target gene expression include, without limitation, examination of protein or mRNA levels using techniques known to those of skill in the art, such as, e.g., dot blots, northern blots, in situ hybridization, ELISA, immunoprecipitation, enzyme function, as well as phenotypic assays known to those of skill in the art.

An “effective amount” or “therapeutically effective amount” of an active agent or therapeutic agent such as a therapeutic nucleic acid is an amount sufficient to produce the desired effect, e.g., an increase or inhibition of expression of a target sequence in comparison to the normal expression level detected in the absence of the nucleic acid. An increase in expression of a target sequence is achieved when any measurable level is detected in the case of an expression product that is not present in the absence of the nucleic acid. In the case where the expression product is present at some level prior to contact with the nucleic acid, an in increase in expression is achieved when the fold increase in value obtained with a nucleic acid such as mRNA relative to control is about 1.05, 1.1, 1.2, 1.3, 1.4, 1.5, 1.75, 2, 2.5, 3, 4, 5, 6, 7, 8, 9, 10, 15, 20, 25, 30, 40, 50, 75, 100, 250, 500, 750, 1000, 5000, 10000 or greater. Inhibition of expression of a target gene or target sequence is achieved when the value obtained with a nucleic acid such as antisense oligonucleotide relative to the control is about 95%, 90%, 85%, 80%, 75%, 70%, 65%, 60%, 55%, 50%, 45%, 40%, 35%, 30%, 25%, 20%, 15%, 10%, 5%, or 0%. Suitable assays for measuring expression of a target gene or target sequence include, e.g., examination of protein or RNA levels using techniques known to those of skill in the art such as dot blots, northern blots, in situ hybridization, ELISA, immunoprecipitation, enzyme function, fluorescence or luminescence of suitable reporter proteins, as well as phenotypic assays known to those of skill in the art.

The term “nucleic acid” as used herein refers to a polymer containing at least two deoxyribonucleotides or ribonucleotides in either single- or double-stranded form and includes DNA, RNA, and hybrids thereof. DNA may be in the form of antisense molecules, plasmid DNA, cDNA, PCR products, or vectors. RNA may be in the form of small hairpin RNA (shRNA), messenger RNA (mRNA), antisense RNA, miRNA, micRNA, multivalent RNA, dicer substrate RNA or viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, single nucleotide polymorphisms, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)). “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

The term “gene” refers to a nucleic acid (e.g., DNA or RNA) sequence that comprises partial length or entire length coding sequences necessary to produce a polypeptide or precursor polypeptide.

“Gene product,” as used herein, refers to a product of a gene such as an RNA transcript or a polypeptide.

The term “lipid” refers to a group of organic compounds that include, but are not limited to, esters of fatty acids and are generally characterized by being poorly soluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

A “steroid” is a compound comprising the following carbon skeleton:

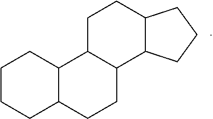 [SMILES: C1CCC2C(C1)CCC1C3CCCC3CCC21]

Non-limiting examples of steroids include cholesterol, and the like.

A “cationic lipid” refers to a lipid capable of being positively charged. Exemplary cationic lipids include one or more amine group(s) which bear the positive charge. Preferred cationic lipids are ionizable such that they can exist in a positively charged or neutral form depending on pH. The ionization of the cationic lipid affects the surface charge of the lipid nanoparticle under different pH conditions. This charge state can influence plasma protein absorption, blood clearance and tissue distribution (Semple, S. C., et al., Adv. Drug Deliv Rev 32:3-17 (1998)) as well as the ability to form endosomolytic non-bilayer structures (Hafez, I. M., et al., Gene Ther 8:1188-1196 (2001)) critical to the intracellular delivery of nucleic acids.

The term “lipid nanoparticle” refers to particles having at least one dimension on the order of nanometers (e.g., 1-1,000 nm) which include one or more of the compounds of Formula (I) or other specified components. In some embodiments, lipid nanoparticles are included in a formulation that can be used to deliver an active agent or therapeutic agent, such as a nucleic acid (e.g., mRNA) to a target site of interest (e.g., cell, tissue, organ, tumor, and the like). In some embodiments, the lipid nanoparticles of the disclosure comprise a nucleic acid. Such lipid nanoparticles typically comprise a compound of Formula (I) and one or more excipient selected from neutral lipids, charged lipids, steroids, and polymer conjugated lipids. In some embodiments, the active agent or therapeutic agent, such as a nucleic acid, may be encapsulated in the lipid portion of the lipid nanoparticle or an aqueous space enveloped by some or all the lipid portion of the lipid nanoparticle, thereby protecting it from enzymatic degradation or other undesirable effects induced by the mechanisms of the host organism or cells, e.g., an adverse immune response.

In various embodiments, the lipid nanoparticles have a mean diameter of from about 30 nm to about 150 nm, from about 40 nm to about 150 nm, from about 40 nm to 100 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. In certain embodiments, nucleic acids, when present in the lipid nanoparticles, are resistant in aqueous solution to degradation with a nuclease. Lipid nanoparticles comprising nucleic acids and their method of preparation are disclosed in, e.g., U.S. Patent Publication Nos. 2004/0142025, 2007/0042031 and PCT Pub. Nos. WO 2013/016058 and WO 2013/086373, the full disclosures of which are herein incorporated by reference in their entirety for all purposes.

As used herein, “lipid encapsulated” refers to a lipid nanoparticle that provides an active agent or therapeutic agent, such as a nucleic acid (e.g., mRNA), with full encapsulation, partial encapsulation, or both. In an embodiment, the nucleic acid (e.g., mRNA) is fully encapsulated in the lipid nanoparticle.

The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-DMG) and the like.

The term “neutral lipid” refers to any of several lipid species that exist either in an uncharged or neutral zwitterionic form at a selected pH. At physiological pH, such lipids include, but are not limited to, phosphotidylcholines such as 1,2-Distearoyl-sn-glycero-3-phosphocholine (DSPC), 1,2-Dipalmitoyl-sn-glycero-3-phosphocholine (DPPC), 1,2-Dimyristoyl-sn-glycero-3-phosphocholine (DMPC), 1-Palmitoyl-2-oleoyl-sn-glycero-3-phosphocholine (POPC), 1,2-dioleoyl-sn-glycero-3-phosphocholine (DOPC), phophatidylethanolamines such as 1,2-Dioleoyl-sn-glycero-3-phosphoethanolamine (DOPE), sphingomyelins (SM), ceramides, steroids such as sterols and their derivatives. Neutral lipids may be synthetic or naturally derived.

The term “charged lipid” refers to any of several lipid species that exist in either a positively charged or negatively charged form independent of the pH within a useful physiological range, e.g., pH ˜3 to pH ˜9. Charged lipids may be synthetic or naturally derived. Examples of charged lipids include phosphatidylserines, phosphatidic acids, phosphatidylglycerols, phosphatidylinositols, sterol hemisuccinates, dialkyl trimethylammonium-propanes, (e.g., DOTAP, DOTMA), dialkyl dimethylaminopropanes, ethyl phosphocholines, dimethylaminoethane carbamoyl sterols (e.g., DC-Chol).

As used herein, the term “aqueous solution” refers to a composition comprising water.

“Serum-stable” in relation to nucleic acid-lipid nanoparticles means that the nucleotide is not significantly degraded after exposure to a serum or nuclease assay that would significantly degrade free DNA or RNA. Suitable assays include, for example, a standard serum assay, a DNAse assay, or an RNAse assay.

“Systemic delivery,” as used herein, refers to delivery of a therapeutic product that can result in a broad exposure of an active agent within an organism. Some techniques of administration can lead to the systemic delivery of certain agents, but not others. Systemic delivery means that a useful, preferably therapeutic, amount of an agent is exposed to most parts of the body. Systemic delivery of lipid nanoparticles can be by any means known in the art including, for example, intravenous, intraarterial, subcutaneous, and intraperitoneal delivery. In some embodiments, systemic delivery of lipid nanoparticles is by intravenous delivery.

“Local delivery,” as used herein, refers to delivery of an active agent directly to a target site within an organism. For example, an agent can be locally delivered by direct injection into a disease site such as a tumor, other target site such as a site of inflammation, or a target organ such as the liver, heart, pancreas, kidney, and the like. Local delivery can also include topical applications or localized injection techniques such as intramuscular, subcutaneous, or intradermal injection. Local delivery does not preclude a systemic pharmacological effect.

“Alkyl” refers to a straight or branched hydrocarbon chain radical consisting solely of carbon and hydrogen atoms that is saturated (i.e., contains no double and/or triple bonds), having from one to twenty-four carbon atoms (C₁-C₂₄ alkyl), one to sixteen carbon atoms (C₁-C₁₆ alkyl), one to twelve carbon atoms (C₁-C₁₂ alkyl), six to twenty-four carbon atoms (C₆-C₂₄ alkyl), one to eight carbon atoms (C₁-C₈ alkyl) or one to six carbon atoms (C₁-C₆ alkyl) and which is attached to the rest of the molecule by a single bond, e.g., methyl, ethyl, n-propyl, 1-methylethyl (iso propyl), n-butyl, n-pentyl, 1,1-dimethylethyl (t-butyl), 3-methylhexyl, 2-methylhexyl, and the like. Unless stated otherwise specifically in the specification, an alkyl group is optionally substituted.

“Alkenyl” refers to a straight or branched hydrocarbon chain radical consisting solely of carbon and hydrogen atoms that contains at least one carbon-carbon double, having from one to twenty-four carbon atoms (C₂-C₂₄ alkenyl), one to twelve carbon atoms (C₂-C₁₂ alkenyl), six to twenty-four carbon atoms (C₆-C₂₄ alkenyl), two to sixteen carbon atoms (C₂-C₁₆ alkenyl), four to twelve carbon atoms (C₄-C₁₂ alkenyl), one to eight carbon atoms (C₂-C₅ alkenyl) or one to six carbon atoms (C₂-C₆ alkenyl) and which is attached to the rest of the molecule by a single bond, e.g., ethenyl, n-propenyl, 1-methylethenyl, n-butenyl, n-pentenyl, 1,1-dimethylethenyl, 3-methylhexenyl, 2-methylhexenyl, and the like. Unless stated otherwise specifically in the specification, an alkenyl group is optionally substituted.

“Alkynyl” refers to a straight or branched hydrocarbon chain radical consisting solely of carbon and hydrogen atoms that contains at least one carbon-carbon triple bond, having from one to twenty-four carbon atoms (C₂-C₂₄ alkynyl), one to twelve carbon atoms (C₂-C₁₂ alkynyl), one to eight carbon atoms (C₂-C₅ alkynyl) or one to six carbon atoms (C₂-C₆ alkynyl) and which is attached to the rest of the molecule by a single bond, e.g., ethynyl, n-propynyl, 1-methylethynyl, n-butynyl, n-pentynyl, 1,1-dimethylethynyl, 3-methylhexynyl, 2-methylhexynyl, and the like. Unless stated otherwise specifically in the specification, an alkynyl group is optionally substituted.

“Alkylene” or “alkylene chain” refers to a straight or branched divalent saturated hydrocarbon chain linking the rest of the molecule to a radical group, consisting solely of carbon and hydrogen. In some embodiments, an alkylene chain has from one to twenty-four carbon atoms (C₁-C₂₄ alkylene), one to fifteen carbon atoms (C₁-C₁₅ alkylene), one to twelve carbon atoms (C₁-C₁₂ alkylene), one to eight carbon atoms (C₁-C₅ alkylene), one to six carbon atoms (C₁-C₆ alkylene), four to six carbon atoms (C₄-C₆ alkylene), two to four carbon atoms (C₂-C₄ alkylene), one to two carbon atoms (C₁-C₂ alkylene), e.g., methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain is optionally substituted.

“Alkenylene” or “alkenylene chain” refers to a straight or branched divalent hydrocarbon chain linking the rest of the molecule to a radical group, consisting solely of carbon and hydrogen and which comprises at least one carbon-carbon double bond. In some embodiments, an alkenylene chain has from two to twenty-four carbon atoms (C₂-C₂₄ alkenylene), two to fifteen carbon atoms (C₂-C₁₅ alkenylene), two to twelve carbon atoms (C₂-C₁₂ alkenylene), two to eight carbon atoms (C₂-C₈ alkenylene), two to six carbon atoms (C₂-C₆ alkenylene), four to six carbon atoms (C₄-C₆ alkenylene), two to four carbon atoms (C₂-C₄ alkenylene), e.g, ethenylene, propenylene, n-butenylene, and the like. The alkenylene chain is attached to the rest of the molecule through a single bond and to the radical group through a single bond. The points of attachment of the alkenylene chain to the rest of the molecule and to the radical group can be through one carbon or any two carbons within the chain. Unless stated otherwise specifically in the specification, an alkenylene chain is optionally substituted.

“Alkoxy” refers to a radical of the formula —ORₐ where Rₐ is an alkyl radical as defined above containing one to twelve carbon atoms (C₁-C₁₂ alkoxy), one to eight carbon atoms (C₁-C₅ alkoxy), or one to six carbon atoms (C₁-C₄ alkoxy), or any value within these ranges. Unless stated otherwise specifically in the specification, an alkoxy group is optionally substituted.

“Cycloalkyl” or “carbocyclic ring” refers to a stable non aromatic monocyclic or polycyclic hydrocarbon radical consisting solely of carbon and hydrogen atoms, which may include fused or bridged ring systems, having from three to fifteen ring carbon atoms (C₃-C₁₅), from three to ten ring carbon atoms (C₃-C₁₀) or from three to eight ring carbon atoms (C₃-C₅), and which is saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl bicyclo[2.2.1]heptanyl, and the like. Unless otherwise stated specifically in the specification, a cycloalkyl group is optionally substituted.

“Aryl” refers to a carbocyclic ring system radical comprising hydrogen, 6 to 18 carbon atoms and at least one aromatic ring. For purposes of this disclosure, the aryl radical is a monocyclic, bicyclic, tricyclic, or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene.

“Arylalkyl” refers to a radical of the formula —R_{b}—R_{c} where R_{b} is an alkylene or alkenylene as defined above and R_{c} is one or more aryl radicals as defined above, for example, benzyl, diphenylmethyl and the like. Unless stated otherwise specifically in the specification, an arylalkyl group is optionally substituted.

“Heterocyclyl” or “heterocyclic ring” refers to a stable 3- to 18-membered non-aromatic ring radical having one to twelve ring carbon atoms (e.g., two to twelve) and from one to six ring heteroatoms selected from the group consisting of nitrogen, oxygen, and sulfur. Unless stated otherwise specifically in the specification, the heterocyclyl radical is a monocyclic, bicyclic, tricyclic, or tetracyclic ring system, which may include fused, spirocyclic (“spiro-heterocyclyl”) and/or bridged ring systems; and the nitrogen, carbon or sulfur atoms in the heterocyclyl radical is optionally oxidized; the nitrogen atom is optionally quaternized; and the heterocyclyl radical is partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group is optionally substituted.

“Heteroaryl” refers to a 5- to 14-membered ring system radical comprising hydrogen atoms, one to thirteen carbon atoms, one to six heteroatoms selected from the group consisting of nitrogen, oxygen, and sulfur, and at least one aromatic ring. For purposes of this disclosure, the heteroaryl radical may be a monocyclic, bicyclic, tricyclic, or tetracyclic ring system, which may include fused or bridged ring systems; and the nitrogen, carbon, or sulfur atoms in the heteroaryl radical may be optionally oxidized; the nitrogen atom may be optionally quaternized. Examples include, but are not limited to, azepinyl, acridinyl, benzimidazolyl, benzthiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, benzoxazolinonyl, benzimidazolthionyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, pteridinonyl, purinyl, pyrrolyl, pyrazolyl, pyridinyl, pyridinonyl, pyrazinyl, pyrimidinyl, pryrimidinonyl, pyridazinyl, pyrrolyl, pyrido[2,3-d]pyrimidinonyl, quinazolinyl, quinazolinonyl, quinoxalinyl, quinoxalinonyl, quinolinyl, isoquinolinyl, tetrahydroquinolinyl, thiazolyl, thiadiazolyl, thieno[3,2-d]pyrimidin-4-onyl, thieno[2,3-d]pyrimidin-4-onyl, triazolyl, tetrazolyl, triazinyl, and thiophenyl (i.e., thienyl). Unless stated otherwise specifically in the specification, a heteroaryl group may be optionally substituted.

The term “substituted” used herein means any of the above groups (e.g., alkyl, alkylhydroxyl, alkenyl, alkynyl, alkylene, cycloalkyl, aryl, aralkyl or heterocyclyl) wherein at least one hydrogen atom is replaced by a bond to a non-hydrogen atoms such as, but not limited to: a halogen atom such as F, Cl, Br, and I; oxo groups (═O); hydroxyl groups (—OH); alkoxy groups (—OR^{a}, where R^{a} is C₁-C₁₂ alkyl or cycloalkyl); carboxyl groups (—OC(═O)R^{a} or —C(═O)OR^{a}, where R^{a} is H, C₁-C₁₂ alkyl or cycloalkyl); amine groups (—NR^{a}R^{b}, where R^{a} and R^{b} are each independently H, C₁-C₁₂ alkyl or cycloalkyl); C₁-C₁₂ alkyl groups; and cycloalkyl groups. In some embodiments the substituent is a C₁-C₁₂ alkyl group. In other embodiments, the substituent is a cycloalkyl group. In other embodiments, the substituent is a halo group, such as fluoro. In other embodiments, the substituent is a oxo group. In other embodiments, the substituent is a hydroxyl group. In other embodiments, the substituent is an alkoxy group. In other embodiments, the substituent is a carboxyl group. In other embodiments, the substituent is an amine group.

“Optional” or “optionally” (e.g., optionally substituted) means that the subsequently described event of circumstances may or may not occur, and that the description includes instances where said event or circumstance occurs and instances in which it does not. For example, “optionally substituted alkyl” means that the alkyl radical may or may not be substituted and that the description includes both substituted alkyl radicals and alkyl radicals having no substitution. In some embodiments, “optionally substituted” means a particular radical is substituted with one or more substituents selected from the group consisting of halo (e.g., F, Cl, Br, and I), oxo (═O), hydroxyl (—OH), alkoxy (—OR^{a}, where R^{a} is C₁-C₁₂ alkyl), cycloalkoxy (—OR^{a}, where R^{a} is C₃-C₈ cycloalkyl), carboxyl (—OC(═O)R^{a} or —C(═O)OR^{a}, where R^{a} is H, C₁-C₁₂ alkyl, or C₃-C₈ cycloalkyl), amine (—NR^{a}R^{b}, where R^{a} and R^{b} are each independently H, C₁-C₁₂ alkyl, or C₃-C₈ cycloalkyl), C₁-C₁₂ alkyl, and C₃-C₈ cycloalkyl.

In some embodiments, “optionally substituted” means substituted with one or more halo substituents. In some embodiments, “optionally substituted” means substituted with one or more oxo substituents. In some embodiments, “optionally substituted” means substituted with one or more hydroxyl substituents. In certain embodiments, “optionally substituted” means substituted with one or more alkoxy substituents. In some embodiments, “optionally substituted” means substituted with one or more cycloalkoxy substituents. In certain embodiments, “optionally substituted” means substituted with one or more carboxy substituents. In some embodiments, “optionally substituted” means substituted with one or more amine substituents. In certain embodiments, “optionally substituted” means substituted with one or more C₁-C₁₂ alkyl substituents. In some embodiments, “optionally substituted” means substituted with one or more C₃-C₈ cycloalkyl substituents.

When a functional group is described as “optionally substituted,” and in turn, substituents on the functional group are also “optionally substituted” and so on, for the purposes of this disclosure, such iterations are limited to five, preferably such iterations are limited to two. In some embodiments, such iterations are limited to one. In some embodiments, such iterations are limited to zero.

This disclosure is also meant to encompass all pharmaceutically acceptable compounds of the compound of Formula (I) being isotopically labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabeled compounds could be useful to help determine or measure the effectiveness of the compounds, by characterizing, for example, the site or mode of action, or binding affinity to pharmacologically important site of action. Certain isotopically labelled compounds of Formula (I), for example, those incorporating a radioactive isotope, are useful in drug and/or substrate tissue distribution studies. The radioactive isotopes tritium, i.e., ³H, and carbon-14, i.e., ¹⁴C, are particularly useful for this purpose in view of their ease of incorporation and ready means of detection.

Substitution with heavier isotopes such as deuterium, i.e., ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O, and ¹³N, can be useful in Positron Emission Topography (PET) studies for examining substrate receptor occupancy. Isotopically labeled compounds of Formula (I) can generally be prepared by conventional techniques known to those skilled in the art or by processes analogous to those described in the Preparations and Examples as set out below using an appropriate isotopically labeled reagent in place of the non-labeled reagent previously employed.

This disclosure is also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the disclosure includes compounds produced by a process comprising administering a compound of this disclosure to a mammal for a period sufficient to yield a metabolic product thereof. Such products are typically identified by administering a radiolabeled compound of the disclosure in a detectable dose to an animal, such as rat, mouse, guinea pig, monkey, or to human, allowing sufficient time for metabolism to occur, and isolating its conversion products from the urine, blood, or other biological samples.

“Stable compound” and “stable structure” are meant to indicate a compound that is sufficiently robust to survive isolation to a useful degree of purity from a reaction mixture, and formulation into an efficacious therapeutic agent.

“Mammal” includes humans and both domestic animals such as laboratory animals and household pets (e.g., cats, dogs, swine, cattle, sheep, goats, horses, rabbits), and non-domestic animals such as wildlife and the like.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Often crystallizations produce a solvate of the compound of the disclosure. As used herein, the term “solvate” refers to an aggregate that comprises one or more molecules of a compound of the disclosure with one or more molecules of solvent. The solvent may be water, in which case the solvate may be a hydrate. Alternatively, the solvent may be an organic solvent. Thus, the compounds of the present disclosure may exist as a hydrate, including a monohydrate, dihydrate, hemihydrate, sesquihydrate, trihydrate, tetrahydrate and the like, as well as the corresponding solvated forms. The compound of the disclosure may be true solvates, while in other cases, the compound of the disclosure may merely retain adventitious water or be a mixture of water plus some adventitious solvent.

A “pharmaceutical composition” refers to a formulation of a compound of the disclosure and a medium generally accepted in the art for the delivery of the biologically active compound to mammals, e.g., humans. Such a medium includes all pharmaceutically acceptable carriers, diluents, or excipients therefor.

“Effective amount” or “therapeutically effective amount” refers to that amount of a compound of the disclosure which, when administered to a mammal, preferably a human, is sufficient to effect treatment in the mammal, preferably a human. The amount of a lipid nanoparticle of the disclosure which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

“Treating” or “treatment” as used herein covers the treatment of the disease or condition of interest in a mammal, preferably a human, having the disease or condition of interest, and includes:
(i) preventing the disease or condition from occurring in a mammal, in particular, when such mammal is predisposed to the condition but has not yet been diagnosed as having it;
(ii) inhibiting the disease or condition, i.e., arresting its development;
(iii) relieving the disease or condition, i.e., causing regression of the disease or condition; or
(iv) relieving the symptoms resulting from the disease or condition, i.e., relieving pain without addressing the underlying disease or condition. As used herein, the terms “disease” and “condition” may be used interchangeably or may be different in that the malady or condition may not have a known causative agent (so that etiology has not yet been worked out) and it is therefore not yet recognized as a disease but only as an undesirable condition or syndrome, wherein a more or less specific set of symptoms have been identified by clinicians.

The compounds of the disclosure, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high-pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are non-superimposable mirror images of one another.

A “tautomer” refers to a proton shift from one atom of a molecule to another atom of the same molecule. The present disclosure includes tautomers of any said compounds.

### Compounds

In an aspect, the disclosure provides novel lipid compounds which can combine with other lipid components such as neutral lipids, charged lipids, steroids, and/or polymer conjugated lipids to form lipid nanoparticles with oligonucleotides. Without wishing to be bound by theory, it is thought that these lipid nanoparticles shield oligonucleotides from degradation in the serum and provide for effective delivery of oligonucleotides to cells in vitro and in vivo.

One embodiment provides a compound having a structure of Formula (I):

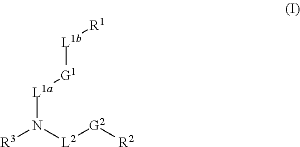

or a pharmaceutically acceptable salt, tautomer, or stereoisomer thereof, wherein:
G¹ is —NR^{1a}—C(═O)— or —C(═O)—NR^{1b}—;
G² is —NR^{2a}—C(═O)— or —C(═O)—NR^{2b}—;
R¹ is —O—C(═O)R^{1c} or —C(═O)—OR^{1c};
R² is C₁-C₁₂ alkyl, C₂-C₁₂ alkenyl, C₂-C₁₂ alkynyl, or -L^{2a}-R^{2c};
R^{1a}, R^{1b}, R^{2a}, and R^{2b} are each independently hydrogen, C₁-C₁₂ alkyl, C₂-C₁₂ alkenyl, or C₂-C₁₂ alkynyl;
R^{1c} is C₆-C₂₄ alkyl, C₆-C₂₄ alkenyl, or C₆-C₂₄ alkynyl;
R^{2c} is —O—C(═O)R^{2d} or —C(═O)—OR^{2d};
R^{2d} is C₁-C₂₄ alkyl, C₂-C₂₄ alkenyl, or C₂-C₂₄ alkynyl;
R³ is C₁-C₈ alkyl or C₃-C₈ cycloalkyl, each of which is optionally substituted with one or more substituents selected from the group consisting of oxo, —OH, —N(R^{3a})R^{3b}, cycloalkyl, heterocyclyl, or heteroaryl;
R^{3a} and R^{3b} are each independently hydrogen, C₁-C₄ alkyl, or C₃-C₈ cycloalkyl;
L^{1a} and L² are each independently C₄-C₁₂ alkylene;
L^{1b} is C₁-C₁₂ alkylene, C₂-C₁₂ alkenylene, or C₂-C₁₂ alkynylene; and
L^{2a} is C₁-C₁₂ alkylene, C₂-C₁₂ alkenylene, or C₂-C₁₂ alkynylene,
wherein each alkyl, alkenyl, alkynyl, alkylene, alkenylene, alkynylene, cycloalkyl, heterocyclyl and heteroaryl is optionally substituted with one or more fluoro.

One embodiment provides a compound having a structure of Formula (I):

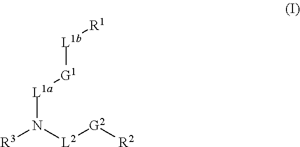

or a pharmaceutically acceptable salt, tautomer, or stereoisomer thereof, wherein:
G¹ is —NR^{1a}—C(═O)—, —C(═O)—NR^{1b}—, or —N(C(═O)R^{1d})—;
G² is —NR^{2a}—C(═O)—, —C(═O)—NR^{2b}—, or —N(C(═O)R^{2e})—;
R¹ is —O—C(═O)R^{1c} or —C(═O)—OR^{1c};
R² is C₁-C₁₂ alkyl, C₂-C₁₂ alkenyl, C₂-C₁₂ alkynyl, or -L^{2a}-R^{2c};
R^{1a}, R^{1b}, R^{2a}, and R^{2b} are each independently hydrogen, C₁-C₁₆ alkyl, C₂-C₁₂ alkenyl, or C₂-C₁₂ alkynyl;
R^{1c} is C₆-C₂₄ alkyl, C₆-C₂₄ alkenyl, or C₆-C₂₄ alkynyl;
R^{2c} is —O—C(═O)R^{2d} or —C(═O)—OR^{2d};
R^{2d} is C₁-C₂₄ alkyl, C₂-C₂₄ alkenyl, or C₂-C₂₄ alkynyl;
R^{1d} and R^{2e} are each independently C₁-C₁₂ alkyl;
R³ is C₁-C₈ alkyl or C₃-C₈ cycloalkyl, each of which is optionally substituted with one or more substituents selected from the group consisting of C₁-C₄ alkoxy, oxo, —OH, —N(R^{3a})R^{3b}, cycloalkyl, heterocyclyl, or heteroaryl;
R^{3a} and R^{3b} are each independently hydrogen, C₁-C₄ alkyl, or C₃-C₈ cycloalkyl;
L^{1a} and L² are each independently C₂-C₁₂ alkylene;
L^{1b} is C₁-C₁₂ alkylene, C₂-C₁₂ alkenylene, or C₂-C₁₂ alkynylene; and
L^{2a} is C₁-C₁₂ alkylene, C₂-C₁₂ alkenylene, or C₂-C₁₂ alkynylene,
wherein each alkyl, alkenyl, alkynyl, alkylene, alkenylene, alkynylene, cycloalkyl, heterocyclyl and heteroaryl is optionally substituted with one or more fluoro.

In some embodiments, G¹ is —NR^{1a}—C(═O)—. In certain embodiments, R^{1a} is hydrogen. In some embodiments, R^{1a} is C₁-C₄ alkyl. In some embodiments, R^{1a} is unsubstituted n-hexyl. In certain embodiments, G¹ is —C(═O)—NR^{1b}—. In some embodiments, R^{1b} is hydrogen. In some embodiments, R^{1b} is C₁-C₄ alkyl (e.g., methyl, ethyl, n-propyl, isopropyl, n-butyl, etc.). In some embodiments, R^{1b} is unsubstituted ethyl. In some embodiments, G¹ is —NH—C(═O)—. In certain embodiments, G¹ is —C(═O)—NH—. In some embodiments, R^{1b} is C₁-C₆ alkyl. In certain embodiments, R^{1b} is unsubstituted methyl, unsubstituted ethyl, unsubstituted isopropyl, or unsubstituted n-hexyl.

In some embodiments, R¹ is —O—C(═O)R^{1c}. In certain embodiments, R¹ is —C(═O)—OR^{1c}. In some embodiments, R^{1c} is C₆-C₂₄ alkyl. In certain embodiments, R^{1c} is branched and unsubstituted C₆-C₂₄ alkyl. In certain embodiments, R^{1c} is branched and unsubstituted C₁₂-Cis alkyl.

In some embodiments, G¹ is —NR^{1a}—C(═O)— or —C(═O)—NR^{1b}— and G² is —NR^{2a}—C(═O)— or —C(═O)—NR^{2b}—. In some embodiments, G¹ is —N(C(═O)R^{1d})—. In certain embodiments, G² is —N(C(═O)R^{2e})—. In some embodiments, G¹ is —N(C(═O)R^{1d})— and G² is —N(C(═O)R^{2e})—.

In some embodiments, G¹ is —N(C(═O)R^{1d})— and R^{1d} is unbranched and unsubstituted C₃, C₅, or C₉ alkyl. In certain embodiments, G² is —N(C(═O)R^{2e})— and R^{2e} is unbranched and unsubstituted C₃, C₅, or C₉ alkyl.

In certain embodiments, R¹ has the following structure:

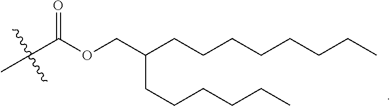 [SMILES: *C(=O)OCC(CCCCCC)CCCCCCCC]

In some embodiments, R¹ has one of the following structures:

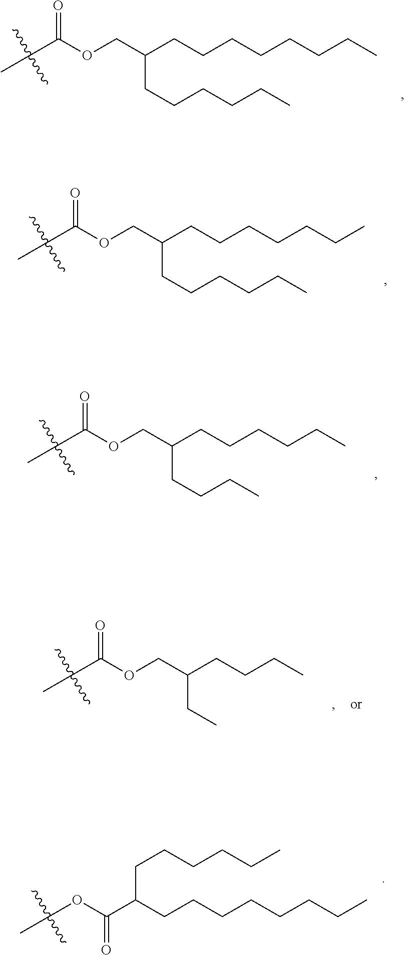 [SMILES: *C(=O)OCC(CCCCCC)CCCCCCCC ; *C(=O)OCC(CCCCCC)CCCCCCC ; *C(=O)OCC(CCCC)CCCCCC ; *C(=O)OCC(CC)CCCC ; *OC(=O)C(CCCCCC)CCCCCCCC]

In certain embodiments, G² is —NR^{2a}—C(═O)—. In some embodiments, R^{2a} is hydrogen. In certain embodiments, R^{2a} is C₁-C₆ alkyl. In some embodiments, R^{2a} is unsubstituted n-hexyl. In some embodiments, R^{2a} is C₁-C₄ alkyl (e.g., methyl, ethyl, n-propyl, isopropyl, n-butyl, etc.). In some embodiments, G² is —C(═O)—NR^{2b}—. In some embodiments, R^{2b} is hydrogen. In some embodiments, R^{2b} is C₁-C₆ alkyl. In certain embodiments, R^{2b} is unsubstituted n-hexyl. In some embodiments, R^{2b} is C₁-C₄ alkyl. In certain embodiments, R^{2b} is unsubstituted ethyl. In some embodiments, R^{2b} is C₁-C₆ alkyl. In certain embodiments, R^{2b} is unsubstituted methyl, unsubstituted ethyl, or unsubstituted isopropyl. In certain embodiments, R^{2b} is unsubstituted methyl, unsubstituted ethyl, unsubstituted isopropyl, or unsubstituted n-hexyl.

In certain embodiments, R^{2b} is C₈-C₁₂ alkyl. In some embodiments, R^{2b} is C₈-C₁₆ alkyl. In certain embodiments, R^{2b} is unbranched and unsubstituted C₈-C₁₂ alkyl. In some embodiments, R^{2b} is unbranched and unsubstituted C₁₀ alkyl. In some embodiments, R^{2b} is unbranched and unsubstituted C₁₀ alkyl or unsubstituted and unbranched C₁₆ alkyl. In certain embodiments, R² is C₁-C₁₂ alkyl or -L^{2a}-R^{2c}. In some embodiments, R² is C₁-C₁₂ alkyl. In some embodiments, R² is C₈-C₁₂ alkyl. In certain embodiments, R² is unbranched and unsubstituted C₈-C₁₂ alkyl. In certain embodiments, R² is unbranched and unsubstituted C₁₀ alkyl.

In certain embodiments, R² is -L^{2a}-R^{2c}. In some embodiments, L^{2a} is C₁-C₁₂ alkylene. In some embodiments, L^{2a} is unbranched and unsubstituted C₁-C₁₂ alkylene (e.g., C₂-C₁₁, C₃-C₁₀, C₄-C₉, C₅-C₈, C₆-C₇, or C₃-C₇ alkylene). In some embodiments, L^{2a} is unsubstituted C₁ or C₂ alkylene. In some embodiments, L^{2a} is unbranched and unsubstituted C₅, C₇, or C₉ alkylene. In certain embodiments, L^{2a} is unbranched and unsubstituted C₅, C₇, C₅, or C₉ alkylene. In some embodiments, L^{2a} is unbranched and unsubstituted C₄, C₅, C₆, C₇, C₈, or C₉ alkylene. In certain embodiments, R^{2c} is —O—C(═O)R^{2d}. In some embodiments, R^{2c} is —C(═O)—OR^{2d}. In some embodiments, R^{2d} is C₁-C₂₄ alkyl. In some embodiments, R^{2d} is C₈-C₁₈ alkyl. In some embodiments, R^{2d} is branched and unsubstituted C₁-C₂₄ alkyl. In certain embodiments, R^{2d} is branched and unsubstituted C₈-C₁₈ alkyl. In certain specific embodiments, R^{2c} has the following structure:

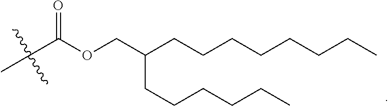 [SMILES: *C(=O)OCC(CCCCCC)CCCCCCCC]

In certain embodiments, R^{2c} has one of the following structures:

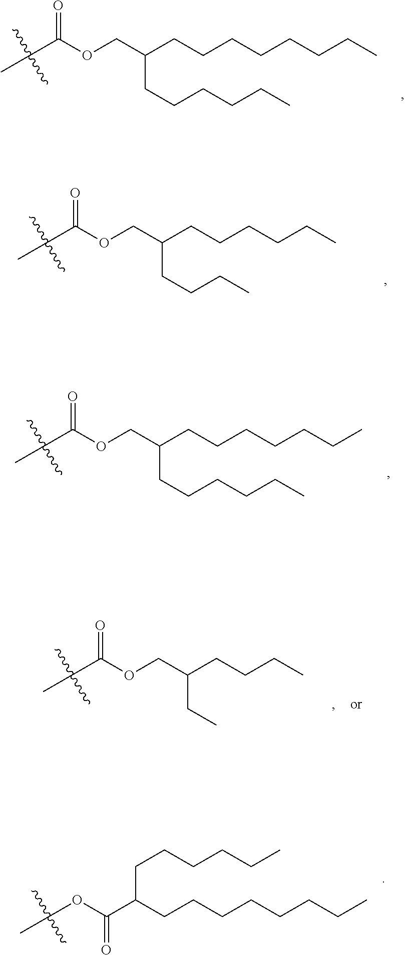 [SMILES: *C(=O)OCC(CCCCCC)CCCCCCCC ; *C(=O)OCC(CCCC)CCCCCC ; *C(=O)OCC(CCCCCC)CCCCCCC ; *C(=O)OCC(CC)CCCC ; *OC(=O)C(CCCCCC)CCCCCCCC]

In some embodiments, R³ is C₁-C₄ alkyl (e.g., methyl, ethyl, n-propyl, isopropyl, n-butyl, etc.). In certain embodiments, R³ is methyl or ethyl optionally substituted with —OH. In some embodiments, R³ is C₁-C₄ alkyl optionally substituted with one or more —OH, 5-6-membered heterocyclyl, 5-6-membered heteroaryl, —N(R^{3a})R^{3b}, or C₁-C₂ alkoxy. In some embodiments, R³ is methyl, ethyl, or n-propyl optionally substituted with —OH, 5-membered heterocyclyl, 5-membered heteroaryl, —N(CH₃)₂, or methoxy.

In some embodiments, R³ is C₃-C₈ cycloalkyl. In certain embodiments, R³ is C₅-C₆ cycloalkyl. In certain embodiments, R³ is C₅-C₆ cycloalkyl substituted with —OH. In some embodiments, R³ is —CH₃ or has one of the following structures:

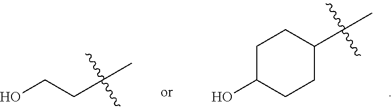

In certain embodiments, R³ is —CH₃ or has one of the following structures:

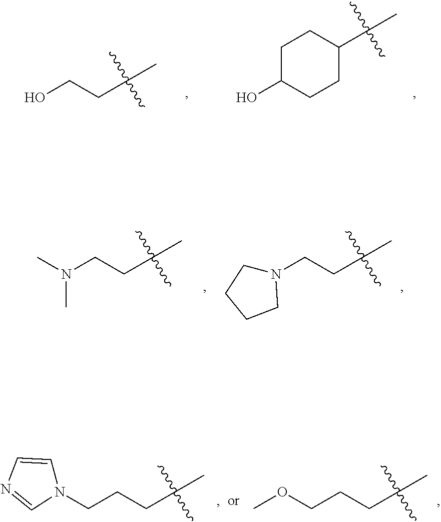

In certain embodiments, L^{1a} is C₃-C₁₂ alkylene. In certain embodiments, L^{1a} is C₂-C₁₂ alkylene. In some embodiments, L² is C₂-C₁₂ alkylene. In some embodiments, L^{1a} is C₃-C₁₂ alkylene and L² is C₂-C₁₂ alkylene.

In some embodiments, L^{1a} is unbranched and unsubstituted C₄-C₁₂ alkylene (e.g., C₄-C₁₁, C₅-C₁₀, C₆-C₉, C₇-C₈ alkylene). In certain embodiments, L² is unbranched and unsubstituted C₄-C₁₂ alkylene (e.g., C₄-C₁₁, C₅-C₁₀, C₆-C₉, C₇-C₈ alkylene). In some embodiments, L^{1a} is unbranched and unsubstituted C₆-C₁₂ alkylene (e.g., C₆-C₁₁, C₇-C₁₀, C₈-C₉ alkylene). In certain embodiments, L² is unbranched and unsubstituted C₆-C₁₂ alkylene (e.g., C₆-C₁₁, C₇-C₁₀, C₈-C₉ alkylene).

In some embodiments, L^{1a} is unbranched and unsubstituted C₂, C₃, C₄, C₅, C₆, C₇, C₈, or C₉ alkylene. In some embodiments, L² is unbranched and unsubstituted C₂, C₃, C₄, C₅, C₆, C₇, C₈, or C₉ alkylene.

In certain embodiments, L^{1a} is unbranched and unsubstituted C₇ alkylene and L² is unbranched and unsubstituted C₂ or C₃ alkylene.

In some embodiments, L^{1a} is unbranched and unsubstituted C₆, C₇, or C₉ alkylene. In certain embodiments, L² is unbranched and unsubstituted C₆, C₇, or C₉ alkylene. In some embodiments, L¹a and L² are both unbranched and unsubstituted C₆ alkylene. In some embodiments, L^{1a} and L² are both unbranched and unsubstituted C₇ alkylene. In certain embodiments, L^{1a} and L² are both unbranched and unsubstituted C₉ alkylene. In some embodiments, L^{1a} is unbranched and unsubstituted C₄, C₅, C₆, C₇, C₈, or C₉ alkylene. In certain embodiments, L² is unbranched and unsubstituted C₄, C₅, C₆, C₇, C₈, or C₉ alkylene.

In certain embodiments, L^{1a} and L² are both unbranched and unsubstituted C₂, C₃, C₄, or C₅ alkylene.

In some embodiments, L^{1a} and L² are both unbranched and unsubstituted C₄ or C₅ alkylene. In certain embodiments, L^{1a} and L² are both unbranched and unsubstituted C₆ or C₇ alkylene. In some embodiments, L^{1a} and L² are both unbranched and unsubstituted C₈ or C₉ alkylene.

In some embodiments, L^{1b} is C₁-C₁₂ alkylene (e.g., C₂-C₁₁, C₃-C₁₀, C₄-C₉, C₅-C₈, C₆-C₇, or C₃-C₇ alkylene). In some embodiments, L^{1b} is unbranched and unsubstituted C₁-C₁₂ alkylene (e.g., C₂-C₁₁, C₃-C₁₀, C₄-C₉, C₅-C₈, C₆-C₇, or C₃-C₇ alkylene). In certain embodiments, L^{1b} is unbranched and unsubstituted C₅, C₇, or C₉ alkylene. In some embodiments, L^{1b} is unbranched and unsubstituted C₁, C₂, C₅, C₆, C₇, C₈, or C₉ alkylene. In some embodiments, L^{1b} is unbranched and unsubstituted C₁, C₂, C₄, C₅, C₆, C₇, C₈, or C₉ alkylene. In certain embodiments, L^{1b} is unsubstituted C₁ or C₂ alkylene. In some embodiments, L^{1b} is unbranched and unsubstituted C₅ alkylene. In certain embodiments, Lib is unbranched and unsubstituted C₅ or C₆ alkylene. In certain embodiments, L^{1b} is unbranched and unsubstituted C₄, C₅, or C₆ alkylene. In some embodiments, L^{1b} is unbranched and unsubstituted C₇ alkylene. In certain embodiments, Lib is unbranched and unsubstituted C₉ alkylene. In some embodiments, L^{1b} is unbranched and unsubstituted C₅, C₇, C₈, or C₉ alkylene. In certain embodiments, L^{1b} is unbranched and unsubstituted C₈ or C₉ alkylene.

In various embodiments, the compound has one of the structures set forth in Table 1 below or a pharmaceutically acceptable salt, tautomer, or stereoisomer thereof.

TABLE 1
Representative compounds of Formula (I)

| No. | Structure |
|---|---|
| I-1 | 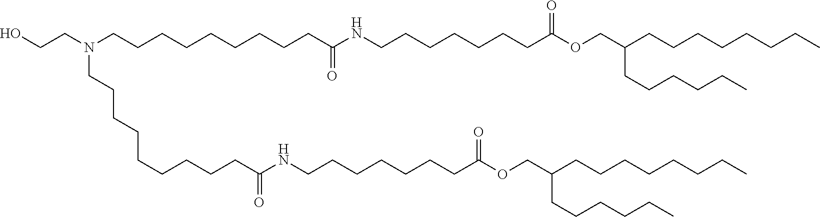 [SMILES: CCCCCCCCC(CCCCCC)COC(=O)CCCCCCCNC(=O)CCCCCCCCCN(CCO)CCCCCCCCCC(=O)NCCCCCCCC(=O)OCC(CCCCCC)CCCCCCCC] |
| I-2 | 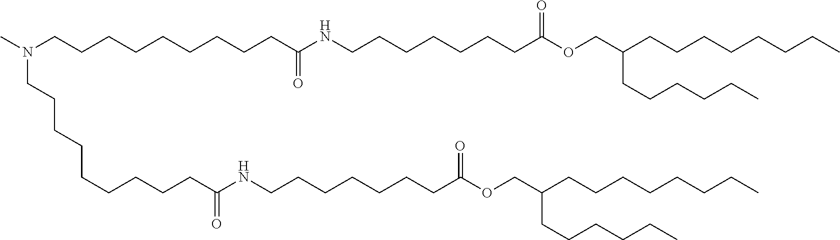 [SMILES: CCCCCCCCC(CCCCCC)COC(=O)CCCCCCCNC(=O)CCCCCCCCCN(C)CCCCCCCCCC(=O)NCCCCCCCC(=O)OCC(CCCCCC)CCCCCCCC] |
| I-3 | 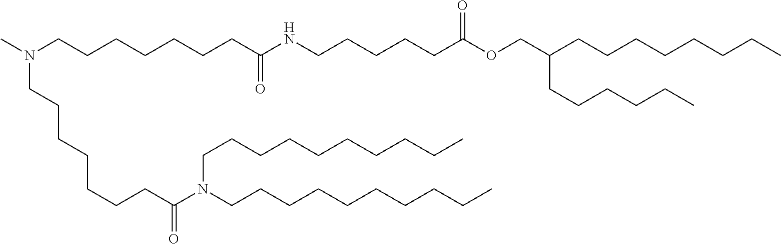 [SMILES: CCCCCCCCCCN(CCCCCCCCCC)C(=O)CCCCCCCN(C)CCCCCCCC(=O)NCCCCCC(=O)OCC(CCCCCC)CCCCCCCC] |
| I-4 | 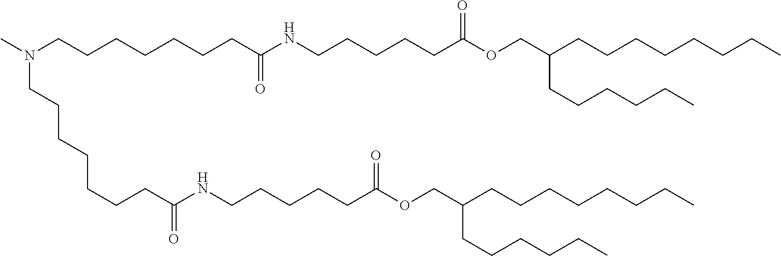 [SMILES: CCCCCCCCC(CCCCCC)COC(=O)CCCCCNC(=O)CCCCCCCN(C)CCCCCCCC(=O)NCCCCCC(=O)OCC(CCCCCC)CCCCCCCC] |
| I-5 | 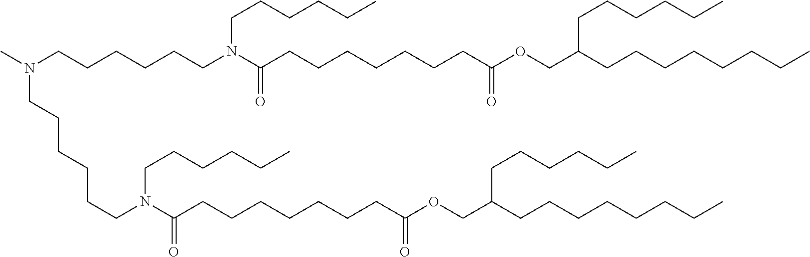 [SMILES: CCCCCCCCC(CCCCCC)COC(=O)CCCCCCCC(=O)N(CCCCCC)CCCCCCN(C)CCCCCCN(CCCCCC)C(=O)CCCCCCCC(=O)OCC(CCCCCC)CCCCCCCC] |
| I-6 | 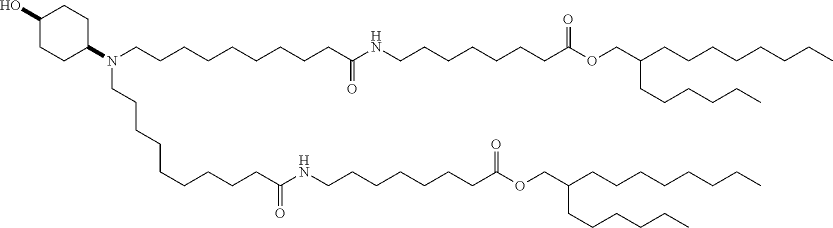 [SMILES: CCCCCCCCC(CCCCCC)COC(=O)CCCCCCCNC(=O)CCCCCCCCCN(CCCCCCCCCC(=O)NCCCCCCCC(=O)OCC(CCCCCC)CCCCCCCC)[C@H]1CC[C@@H](O)CC1] |
| I-7 | 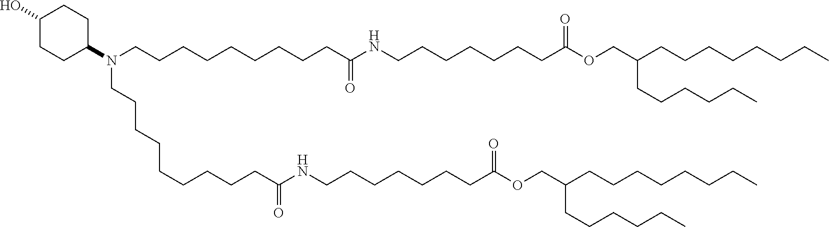 [SMILES: CCCCCCCCC(CCCCCC)COC(=O)CCCCCCCNC(=O)CCCCCCCCCN(CCCCCCCCCC(=O)NCCCCCCCC(=O)OCC(CCCCCC)CCCCCCCC)[C@H]1CC[C@H](O)CC1] |
| I-8 | 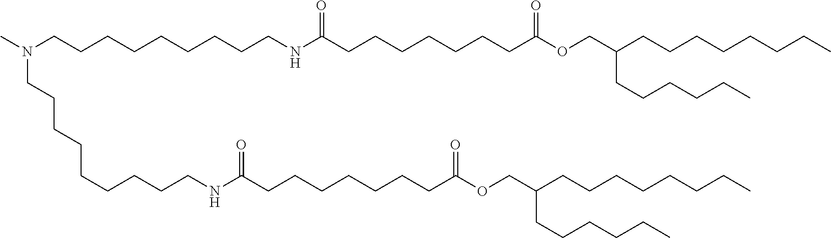 [SMILES: CCCCCCCCC(CCCCCC)COC(=O)CCCCCCCC(=O)NCCCCCCCCCN(C)CCCCCCCCCNC(=O)CCCCCCCC(=O)OCC(CCCCCC)CCCCCCCC] |
| I-9 | 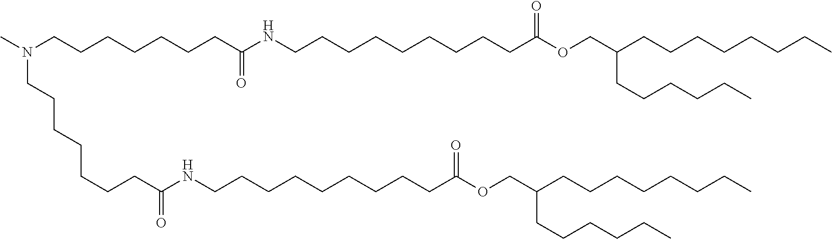 [SMILES: CCCCCCCCC(CCCCCC)COC(=O)CCCCCCCCCNC(=O)CCCCCCCN(C)CCCCCCCC(=O)NCCCCCCCCCC(=O)OCC(CCCCCC)CCCCCCCC] |
| I-10 | 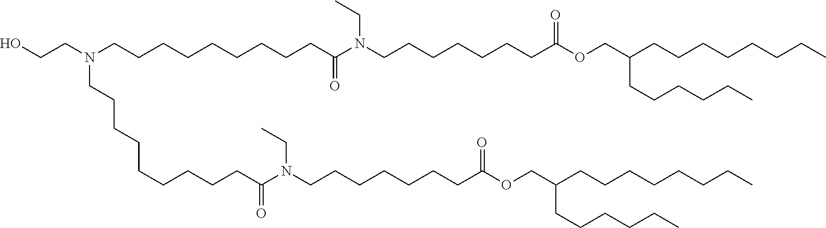 [SMILES: CCCCCCCCC(CCCCCC)COC(=O)CCCCCCCN(CC)C(=O)CCCCCCCCCN(CCO)CCCCCCCCCC(=O)N(CC)CCCCCCCC(=O)OCC(CCCCCC)CCCCCCCC] |
| I-11 | 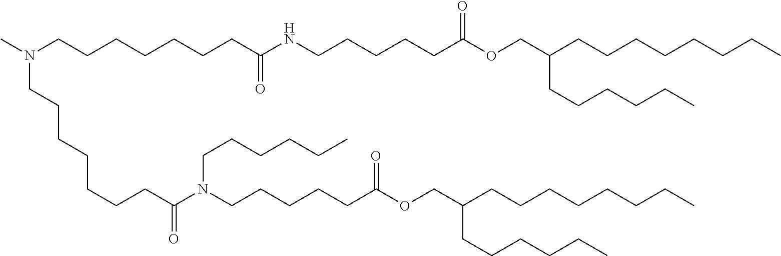 [SMILES: CCCCCCCCC(CCCCCC)COC(=O)CCCCCNC(=O)CCCCCCCN(C)CCCCCCCC(=O)N(CCCCCC)CCCCCC(=O)OCC(CCCCCC)CCCCCCCC] |
| I-12 | 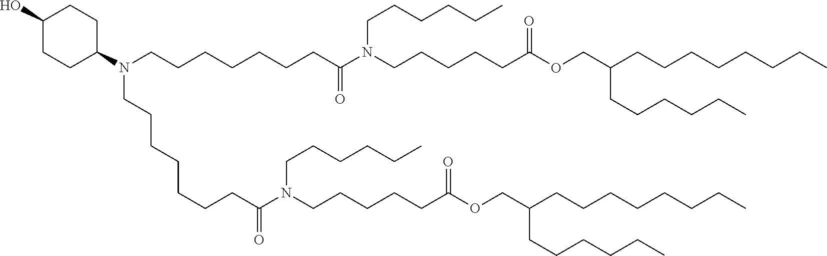 [SMILES: CCCCCCCCC(CCCCCC)COC(=O)CCCCCN(CCCCCC)C(=O)CCCCCCCN(CCCCCCCC(=O)N(CCCCCC)CCCCCC(=O)OCC(CCCCCC)CCCCCCCC)[C@H]1CC[C@@H](O)CC1] |
| I-13 | 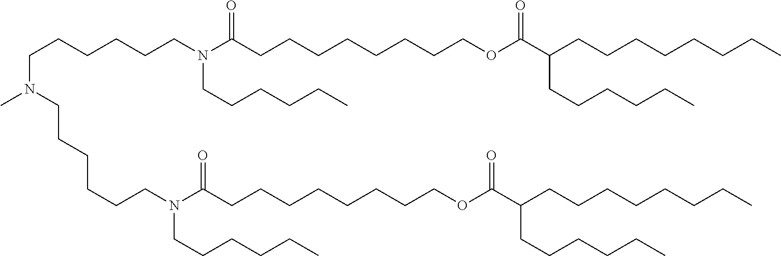 [SMILES: CCCCCCCCC(CCCCCC)C(=O)OCCCCCCCCC(=O)N(CCCCCC)CCCCCCN(C)CCCCCCN(CCCCCC)C(=O)CCCCCCCCOC(=O)C(CCCCCC)CCCCCCCC] |
| I-14 | 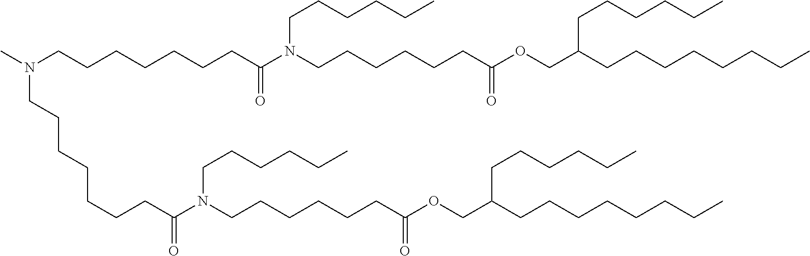 [SMILES: CCCCCCCCC(CCCCCC)COC(=O)CCCCCCN(CCCCCC)C(=O)CCCCCCCN(C)CCCCCCCC(=O)N(CCCCCC)CCCCCCC(=O)OCC(CCCCCC)CCCCCCCC] |
| I-15 | 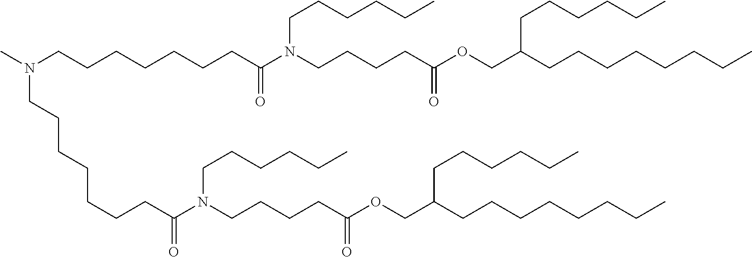 [SMILES: CCCCCCCCC(CCCCCC)COC(=O)CCCCN(CCCCCC)C(=O)CCCCCCCN(C)CCCCCCCC(=O)N(CCCCCC)CCCCC(=O)OCC(CCCCCC)CCCCCCCC] |
| I-16 | 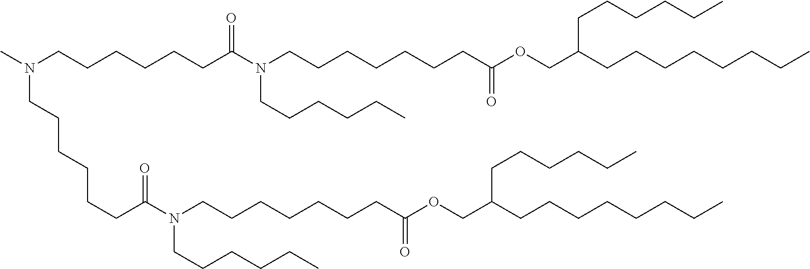 [SMILES: CCCCCCCCC(CCCCCC)COC(=O)CCCCCCCN(CCCCCC)C(=O)CCCCCCN(C)CCCCCCC(=O)N(CCCCCC)CCCCCCCC(=O)OCC(CCCCCC)CCCCCCCC] |
| I-17 | 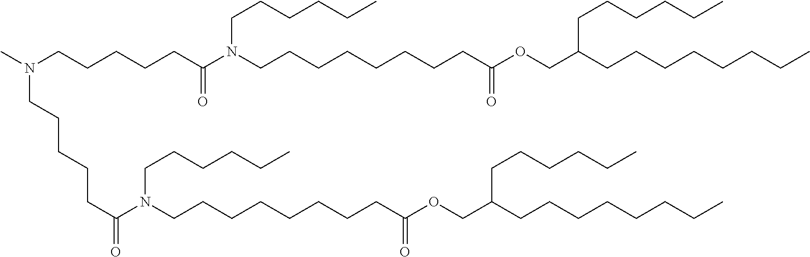 [SMILES: CCCCCCCCC(CCCCCC)COC(=O)CCCCCCCCN(CCCCCC)C(=O)CCCCCN(C)CCCCCC(=O)N(CCCCCC)CCCCCCCCC(=O)OCC(CCCCCC)CCCCCCCC] |
| I-18 | 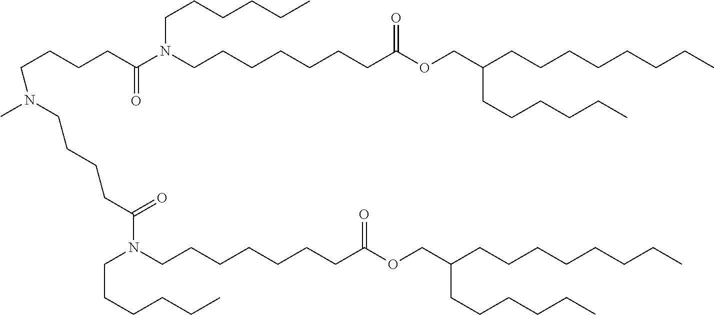 [SMILES: CCCCCCCCC(CCCCCC)COC(=O)CCCCCCCN(CCCCCC)C(=O)CCCCN(C)CCCCC(=O)N(CCCCCC)CCCCCCCC(=O)OCC(CCCCCC)CCCCCCCC] |
| I-19 | 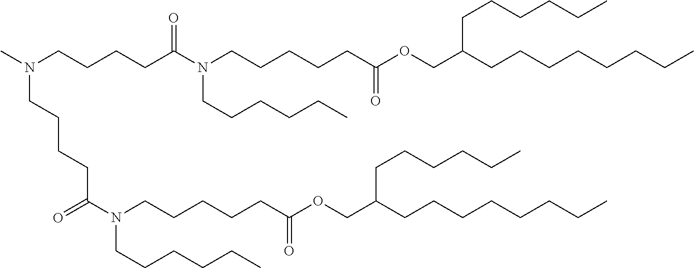 [SMILES: CCCCCCCCC(CCCCCC)COC(=O)CCCCCN(CCCCCC)C(=O)CCCCN(C)CCCCC(=O)N(CCCCCC)CCCCCC(=O)OCC(CCCCCC)CCCCCCCC] |
| I-20 | 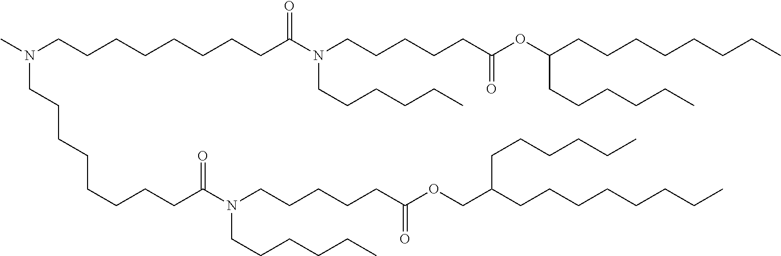 [SMILES: CCCCCCCCC(CCCCCC)COC(=O)CCCCCN(CCCCCC)C(=O)CCCCCCCCN(C)CCCCCCCCC(=O)N(CCCCCC)CCCCCC(=O)OC(CCCCCC)CCCCCCCC] |
| I-21 | 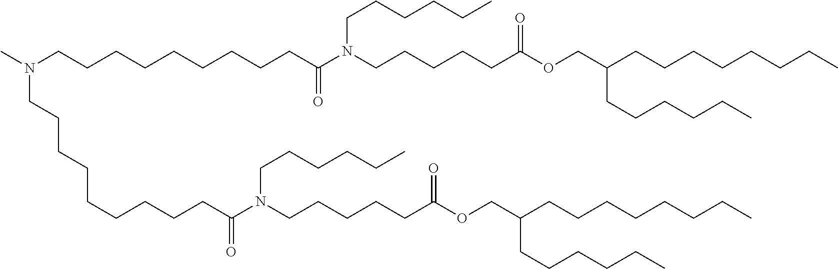 [SMILES: CCCCCCCCC(CCCCCC)COC(=O)CCCCCN(CCCCCC)C(=O)CCCCCCCCCN(C)CCCCCCCCCC(=O)N(CCCCCC)CCCCCC(=O)OCC(CCCCCC)CCCCCCCC] |
| I-22 | 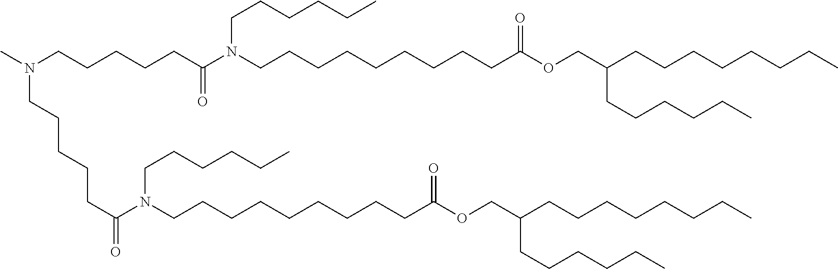 [SMILES: CCCCCCCCC(CCCCCC)COC(=O)CCCCCCCCCN(CCCCCC)C(=O)CCCCCN(C)CCCCCC(=O)N(CCCCCC)CCCCCCCCCC(=O)OCC(CCCCCC)CCCCCCCC] |
| I-23 | 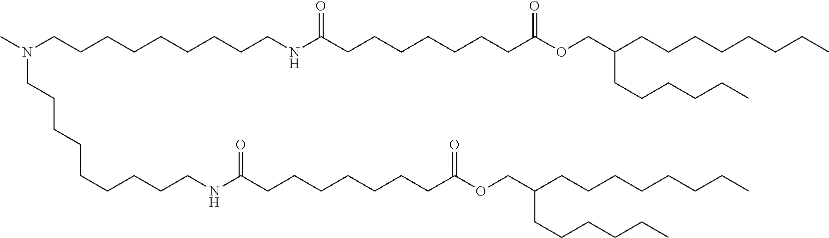 [SMILES: CCCCCCCCC(CCCCCC)COC(=O)CCCCCCCC(=O)NCCCCCCCCCN(C)CCCCCCCCCNC(=O)CCCCCCCC(=O)OCC(CCCCCC)CCCCCCCC] |
| I-24 | 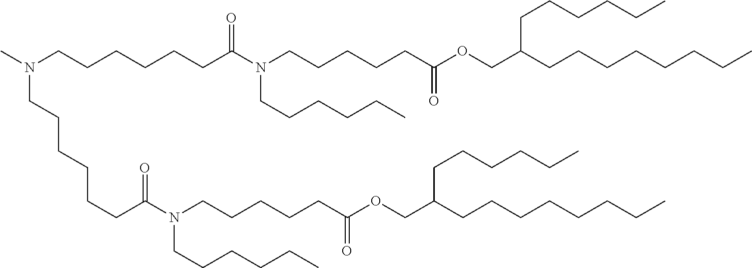 [SMILES: CCCCCCCCC(CCCCCC)COC(=O)CCCCCN(CCCCCC)C(=O)CCCCCCN(C)CCCCCCC(=O)N(CCCCCC)CCCCCC(=O)OCC(CCCCCC)CCCCCCCC] |
| I-25 | 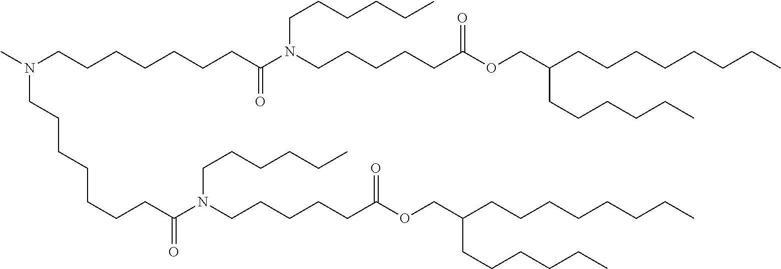 [SMILES: CCCCCCCCC(CCCCCC)COC(=O)CCCCCN(CCCCCC)C(=O)CCCCCCCN(C)CCCCCCCC(=O)N(CCCCCC)CCCCCC(=O)OCC(CCCCCC)CCCCCCCC] |
| I-26 | 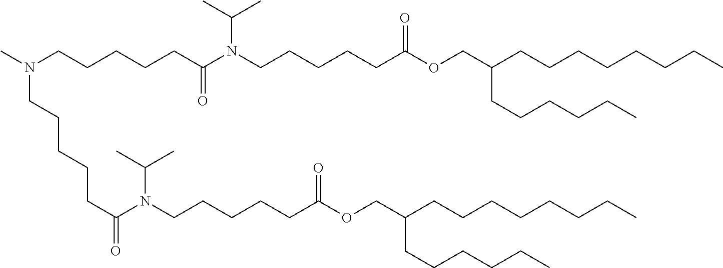 [SMILES: CCCCCCCCC(CCCCCC)COC(=O)CCCCCN(C(=O)CCCCCN(C)CCCCCC(=O)N(CCCCCC(=O)OCC(CCCCCC)CCCCCCCC)C(C)C)C(C)C] |
| I-27 | 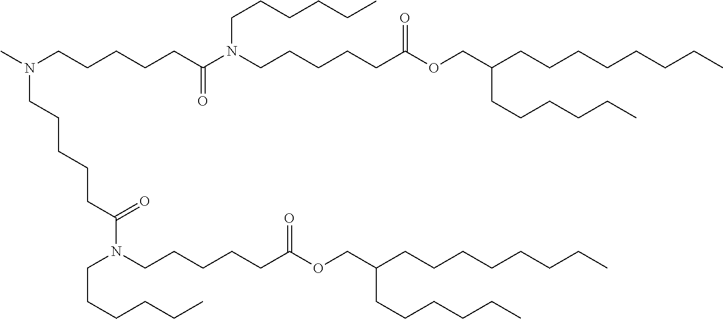 [SMILES: CCCCCCCCC(CCCCCC)COC(=O)CCCCCN(CCCCCC)C(=O)CCCCCN(C)CCCCCC(=O)N(CCCCCC)CCCCCC(=O)OCC(CCCCCC)CCCCCCCC] |
| I-28 | 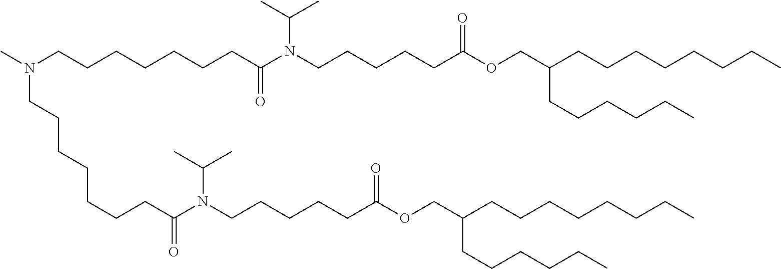 [SMILES: CCCCCCCCC(CCCCCC)COC(=O)CCCCCN(C(=O)CCCCCCCN(C)CCCCCCCC(=O)N(CCCCCC(=O)OCC(CCCCCC)CCCCCCCC)C(C)C)C(C)C] |
| I-29 | 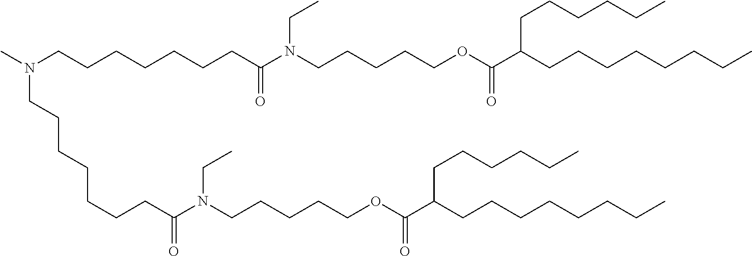 [SMILES: CCCCCCCCC(CCCCCC)C(=O)OCCCCCN(CC)C(=O)CCCCCCCN(C)CCCCCCCC(=O)N(CC)CCCCCOC(=O)C(CCCCCC)CCCCCCCC] |
| I-30 | 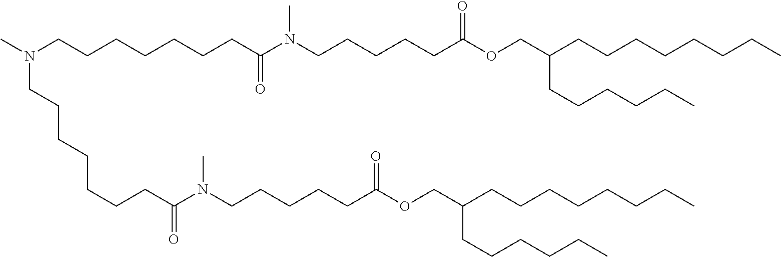 [SMILES: CCCCCCCCC(CCCCCC)COC(=O)CCCCCN(C)C(=O)CCCCCCCN(C)CCCCCCCC(=O)N(C)CCCCCC(=O)OCC(CCCCCC)CCCCCCCC] |
| I-31 | 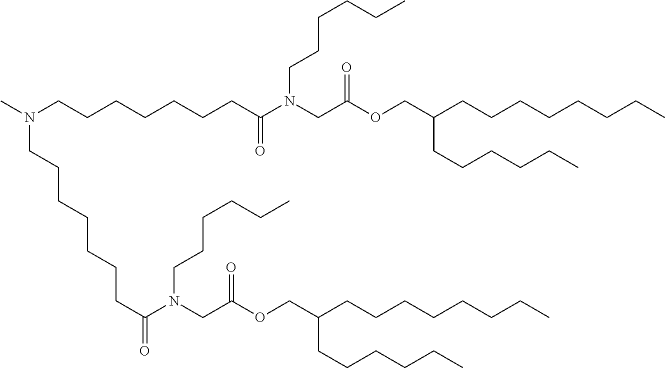 [SMILES: CCCCCCCCC(CCCCCC)COC(=O)CN(CCCCCC)C(=O)CCCCCCCN(C)CCCCCCCC(=O)N(CCCCCC)CC(=O)OCC(CCCCCC)CCCCCCCC] |
| I-32 | 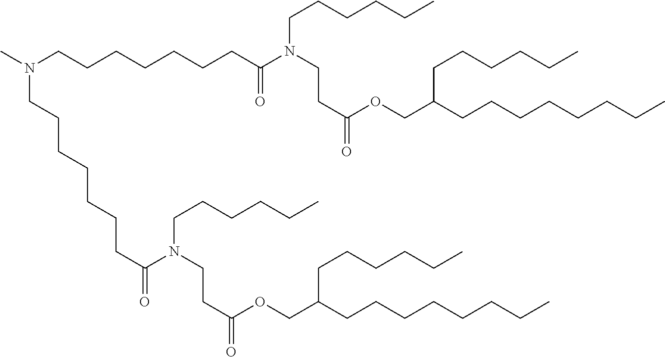 [SMILES: CCCCCCCCC(CCCCCC)COC(=O)CCN(CCCCCC)C(=O)CCCCCCCN(C)CCCCCCCC(=O)N(CCCCCC)CCC(=O)OCC(CCCCCC)CCCCCCCC] |
| I-33 | 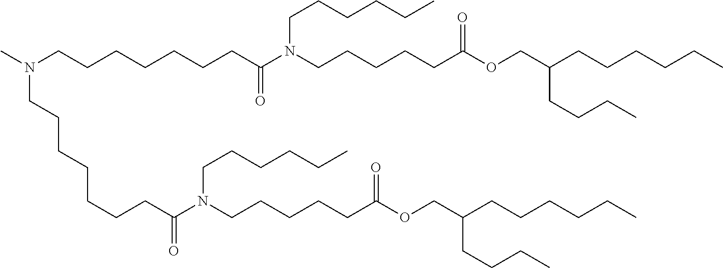 [SMILES: CCCCCCC(CCCC)COC(=O)CCCCCN(CCCCCC)C(=O)CCCCCCCN(C)CCCCCCCC(=O)N(CCCCCC)CCCCCC(=O)OCC(CCCC)CCCCCC] |
| I-34 | 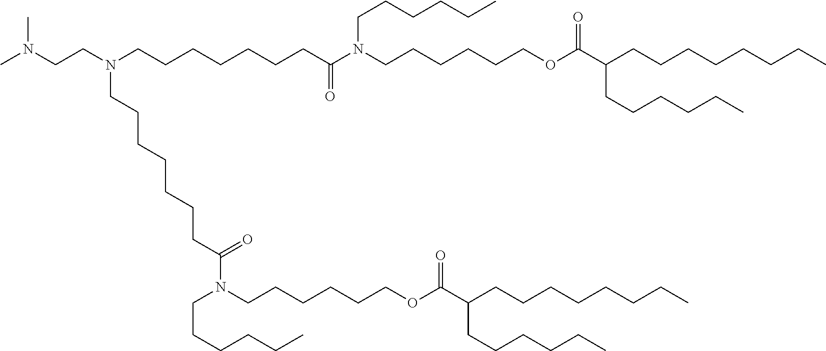 [SMILES: CCCCCCCCC(CCCCCC)C(=O)OCCCCCCN(CCCCCC)C(=O)CCCCCCCN(CCCCCCCC(=O)N(CCCCCC)CCCCCCOC(=O)C(CCCCCC)CCCCCCCC)CCN(C)C] |
| I-35 | 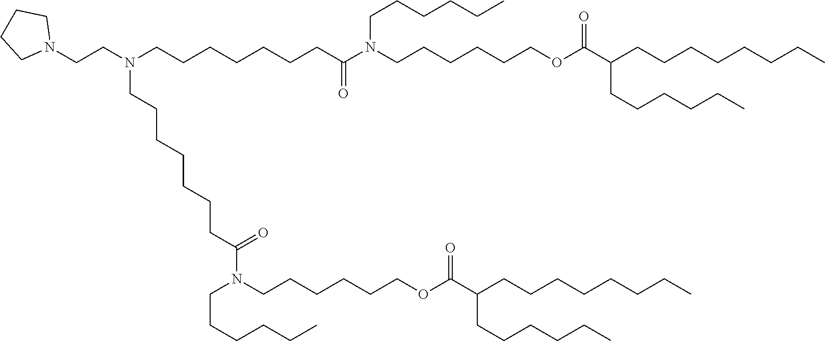 [SMILES: CCCCCCCCC(CCCCCC)C(=O)OCCCCCCN(CCCCCC)C(=O)CCCCCCCN(CCCCCCCC(=O)N(CCCCCC)CCCCCCOC(=O)C(CCCCCC)CCCCCCCC)CCN1CCCC1] |
| I-36 | 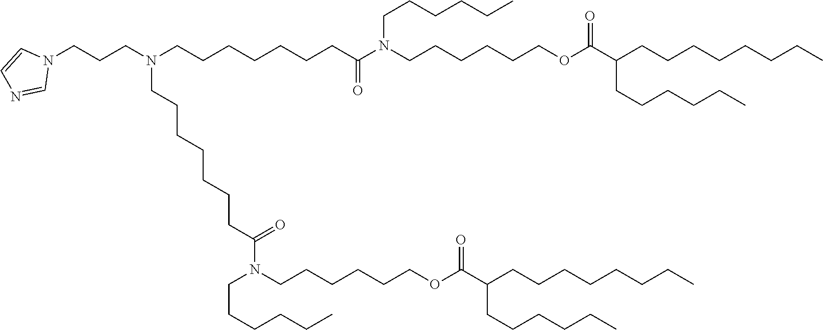 [SMILES: CCCCCCCCC(CCCCCC)C(=O)OCCCCCCN(CCCCCC)C(=O)CCCCCCCN(CCCCCCCC(=O)N(CCCCCC)CCCCCCOC(=O)C(CCCCCC)CCCCCCCC)CCCn1ccnc1] |
| I-37 | 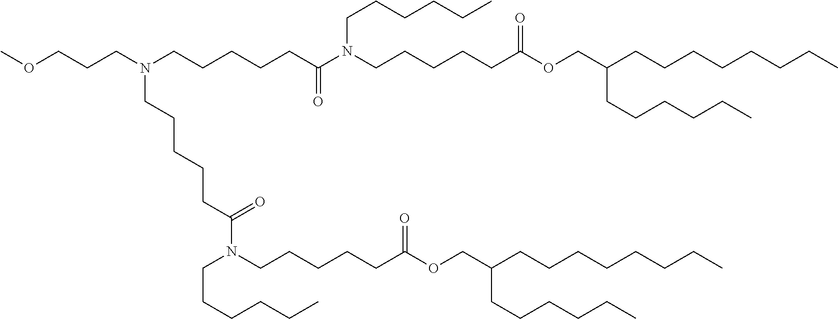 [SMILES: CCCCCCCCC(CCCCCC)COC(=O)CCCCCN(CCCCCC)C(=O)CCCCCN(CCCCCC(=O)N(CCCCCC)CCCCCC(=O)OCC(CCCCCC)CCCCCCCC)CCCOC] |
| I-38 | 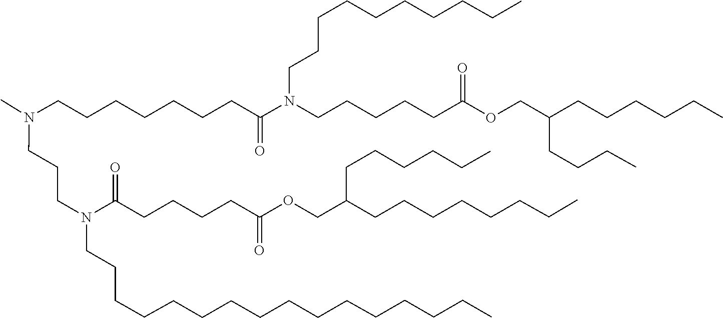 [SMILES: CCCCCCCCCCCCCCCCN(CCCN(C)CCCCCCCC(=O)N(CCCCCCCCCC)CCCCCC(=O)OCC(CCCC)CCCCCC)C(=O)CCCCC(=O)OCC(CCCCCC)CCCCCCCC] |
| I-39 | 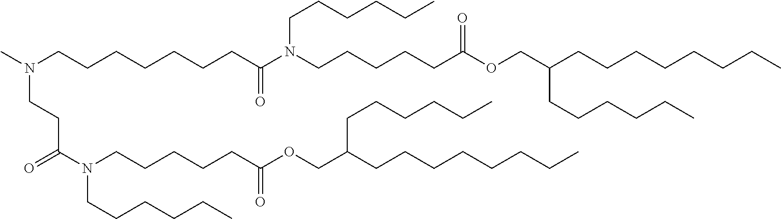 [SMILES: CCCCCCCCC(CCCCCC)COC(=O)CCCCCN(CCCCCC)C(=O)CCCCCCCN(C)CCC(=O)N(CCCCCC)CCCCCC(=O)OCC(CCCCCC)CCCCCCCC] |
| I-40 | 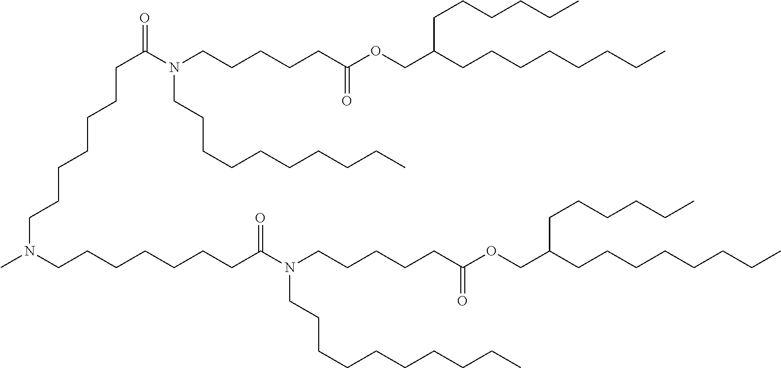 [SMILES: CCCCCCCCCCN(CCCCCC(=O)OCC(CCCCCC)CCCCCCCC)C(=O)CCCCCCCN(C)CCCCCCCC(=O)N(CCCCCCCCCC)CCCCCC(=O)OCC(CCCCCC)CCCCCCCC] |
| I-41 | 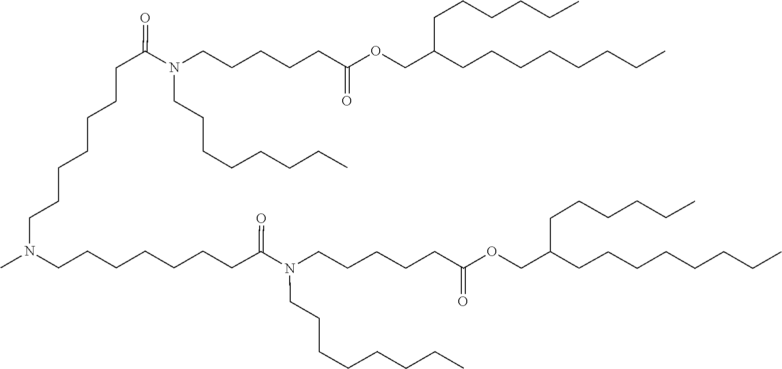 [SMILES: CCCCCCCCC(CCCCCC)COC(=O)CCCCCN(CCCCCCCC)C(=O)CCCCCCCN(C)CCCCCCCC(=O)N(CCCCCCCC)CCCCCC(=O)OCC(CCCCCC)CCCCCCCC] |
| I-42 | 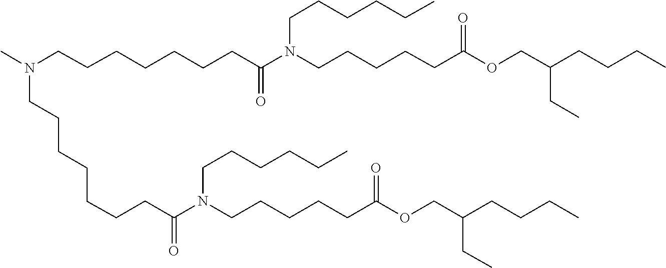 [SMILES: CCCCCCN(CCCCCC(=O)OCC(CC)CCCC)C(=O)CCCCCCCN(C)CCCCCCCC(=O)N(CCCCCC)CCCCCC(=O)OCC(CC)CCCC] |
| I-43 | 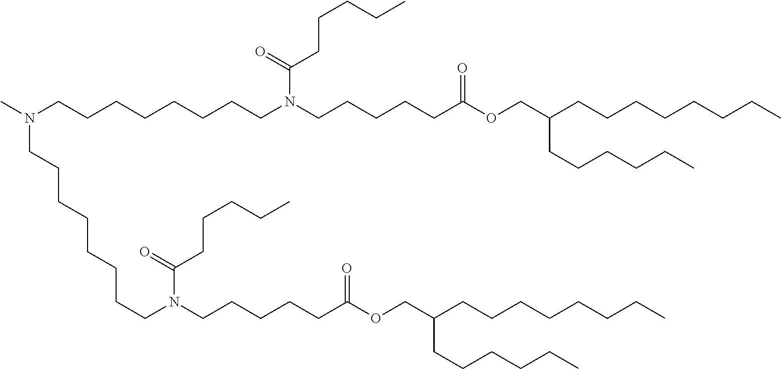 [SMILES: CCCCCCCCC(CCCCCC)COC(=O)CCCCCN(CCCCCCCCN(C)CCCCCCCCN(CCCCCC(=O)OCC(CCCCCC)CCCCCCCC)C(=O)CCCCC)C(=O)CCCCC] |
| I-44 | 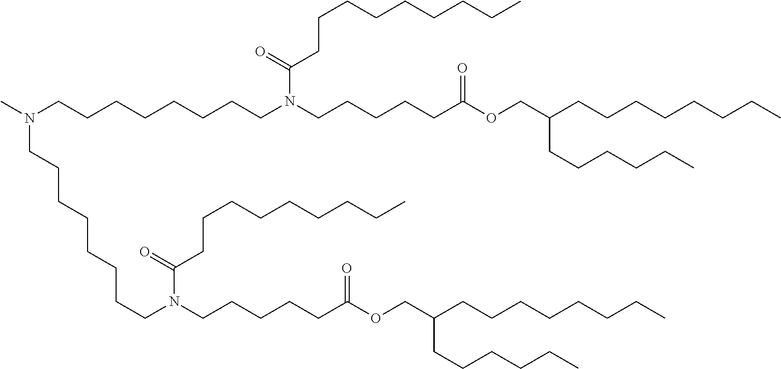 |
| I-45 | 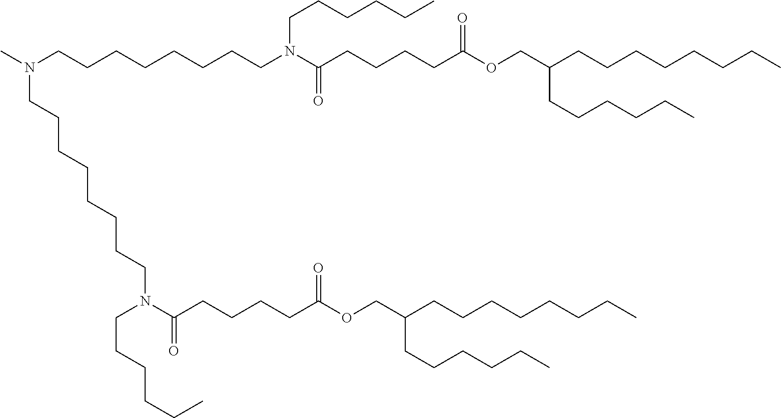 [SMILES: CCCCCCCCC(CCCCCC)COC(=O)CCCCC(=O)N(CCCCCC)CCCCCCCCN(C)CCCCCCCCN(CCCCCC)C(=O)CCCCC(=O)OCC(CCCCCC)CCCCCCCC] |
| I-46 | 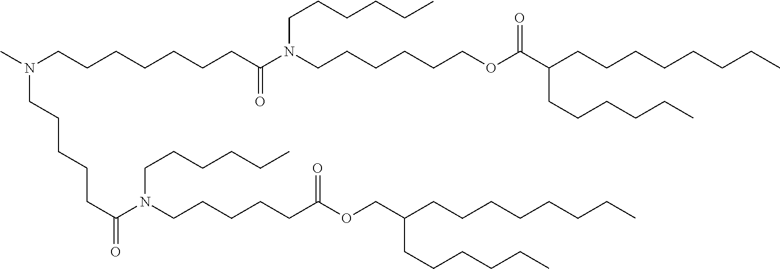 [SMILES: CCCCCCCCC(CCCCCC)COC(=O)CCCCCN(CCCCCC)C(=O)CCCCCN(C)CCCCCCCC(=O)N(CCCCCC)CCCCCCOC(=O)C(CCCCCC)CCCCCCCC] |
| I-47 | 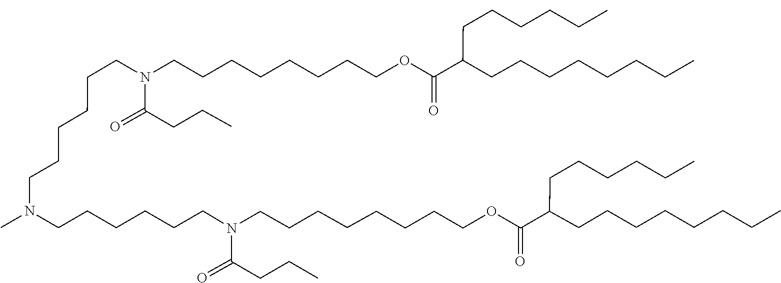 |
| I-48 | 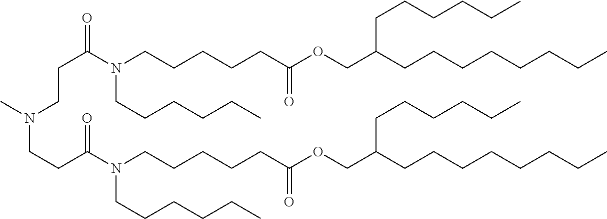 [SMILES: CCCCCCCCC(CCCCCC)COC(=O)CCCCCN(CCCCCC)C(=O)CCN(C)CCC(=O)N(CCCCCC)CCCCCC(=O)OCC(CCCCCC)CCCCCCCC] |
| I-49 | 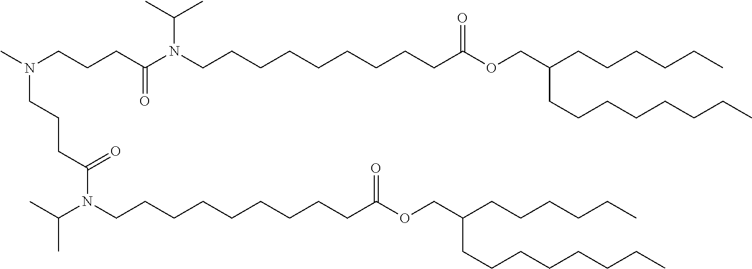 [SMILES: CCCCCCCCC(CCCCCC)COC(=O)CCCCCCCCCN(C(=O)CCCN(C)CCCC(=O)N(CCCCCCCCCC(=O)OCC(CCCCCC)CCCCCCCC)C(C)C)C(C)C] |
| I-50 | 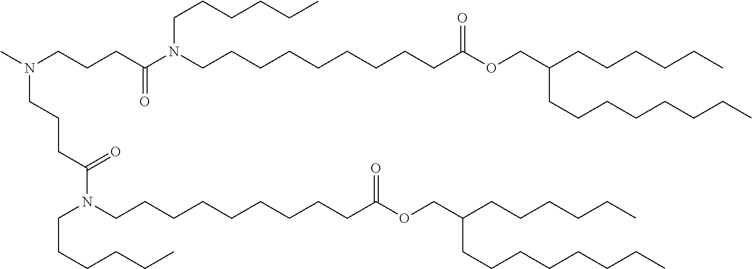 [SMILES: CCCCCCCCC(CCCCCC)COC(=O)CCCCCCCCCN(CCCCCC)C(=O)CCCN(C)CCCC(=O)N(CCCCCC)CCCCCCCCCC(=O)OCC(CCCCCC)CCCCCCCC] |
| I-51 | 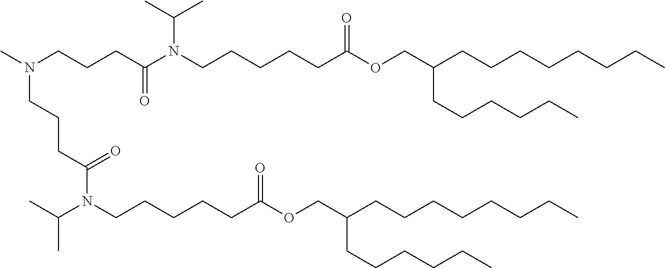 [SMILES: CCCCCCCCC(CCCCCC)COC(=O)CCCCCN(C(=O)CCCN(C)CCCC(=O)N(CCCCCC(=O)OCC(CCCCCC)CCCCCCCC)C(C)C)C(C)C] |
| I-52 | 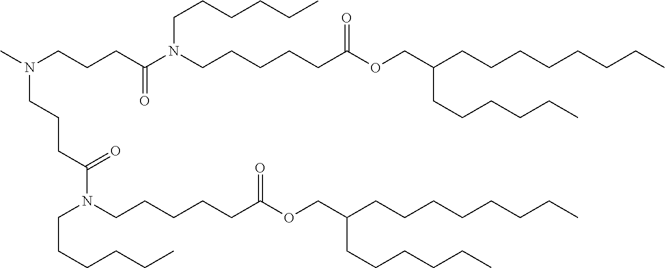 [SMILES: CCCCCCCCC(CCCCCC)COC(=O)CCCCCN(CCCCCC)C(=O)CCCN(C)CCCC(=O)N(CCCCCC)CCCCCC(=O)OCC(CCCCCC)CCCCCCCC] |

It is understood that any embodiment of the compounds of Formula (I), as set forth above, and any specific substituent and/or variable in the compound Formula (I), as set forth above, may be independently combined with other embodiments and/or substituents and/or variables of compounds of Formula (I) to form embodiments of the disclosure not specifically set forth above. In addition, in the event that a list of substituents and/or variables is listed for any particular group (e.g., an R group or L group) in a particular embodiment and/or claim, it is understood that each individual substituent and/or variable may be deleted from the particular embodiment and/or claim and that the remaining list of substituents and/or variables will be considered to be within the scope of embodiments of the disclosure.

It is understood that in the present description, combinations of substituents and/or variables of the depicted formulae are permissible only if such contributions result in stable compounds.

For the purposes of administration, the compounds of the present disclosure (typically in the form of lipid nanoparticles in combination with a therapeutic agent) may be administered as a raw chemical or may be formulated as pharmaceutical compositions. Pharmaceutical compositions of the present disclosure comprise a compound of Formula (I) and one or more pharmaceutically acceptable carrier, diluent, or excipient. The compound of Formula (I) is present in the composition in an amount which is effective to form a lipid nanoparticle and deliver the therapeutic agent, e.g., for treating a particular disease or condition of interest. Appropriate concentrations and dosages can be readily determined by one skilled in the art.

An embodiment provides a composition (e.g., a lipid nanoparticle) comprising a compound of Formula (I) and a therapeutic agent. In some embodiments, the composition (e.g., a lipid nanoparticle) further comprises one or more excipient selected from neutral lipids, steroids, and polymer conjugated lipids.

In some embodiments, the therapeutic agent comprises a nucleic acid. In certain embodiments, the nucleic acid is selected from antisense and messenger RNA.

In certain embodiments, the composition (e.g., a lipid nanoparticle) comprises one or more neutral lipids selected from DSPC, DPPC, DMPC, DOPC, POPC, DOPE, and SM. In some embodiments, the neutral lipid is DSPC. In some embodiments, the molar ratio of the compound to the neutral lipid ranges from about 2:1 to about 8:1.

In certain embodiments, the steroid is cholesterol. In some embodiments, the molar ratio of the compound to cholesterol ranges from about 2:1 to 1:1. In some embodiments, the molar ratio of the compound to cholesterol ranges from 5:1 to 1:1 or from 2:1 to 1:1.

In certain embodiments, the polymer conjugated lipid is a pegylated lipid. In various embodiments, the polymer conjugated lipid is a pegylated lipid. For example, some embodiments include a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a pegylated ceramide (PEG-cer), or a PEG dialkoxypropylcarbamate such as ω-methoxy(polyethoxy)ethyl-N-(2,3-di(tetradecanoxy)propyl)carbamate or 2,3-di(tetradecanoxy)propyl-N-(ω-methoxy(polyethoxy)ethyl)carbamate. In some embodiments, the molar ratio of the compound to the pegylated lipid ranges from about 100:1 to about 10:1 or from about 100:1 to about 25:1. In some embodiments, the molar ratio of the compound to pegylated lipid ranges from about 100:1 to about 20:1 or from about 100:1 to about 10:1. In some embodiments, the pegylated lipid is PEG-DMG.

In certain embodiments, the pegylated lipid has the following Formula (II):

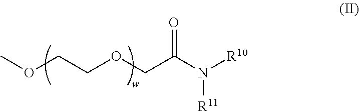

or a pharmaceutically acceptable salt, tautomer, or stereoisomer thereof, wherein:
R¹⁰ and R¹¹ are each independently a straight or branched, alkyl, alkenyl or alkynyl from 10 to 30 carbon atoms, wherein the alkyl, alkenyl or alkynyl is optionally interrupted by one or more ester bonds; and
w has a mean value ranging from 30 to 60.

In certain embodiments, R¹⁰ and R¹¹ are each independently straight alkyl chain containing from 12 to 16 carbon atoms. In some embodiments, the average w is about 45, 46, 47, 48, 49, 50, 51, 52, 53, 54, or 55. In some embodiments, the average w is about 49. In certain embodiments, w has a value ranging from 30 to 60. In some embodiments, w ranges from 40-50. In some embodiments, w is 40, 41, 42, 43, 44, 45, 46, 47, 48, 49, or 50.

In some embodiments, the lipid nanoparticle or composition comprises a plurality of pegylated lipids of Formula (II) and the average w for the plurality ranges from 40-50. In some embodiments, the average w is 43, 44, 45, 46, 47, or 48.

Synthesis of pegylated lipids can be found in U.S. Pat. No. 9,738,593, the disclosure of which is hereby incorporated by reference.

Administration of the compositions of the disclosure can be carried out via any of the accepted modes of administration of agents for serving similar utilities. The pharmaceutical compositions of the disclosure may be formulated into preparations in solid, semi-solid, liquid, or gaseous forms, such as tablets, capsules, powders, granules, ointments, solutions, suspensions, suppositories, injections, inhalants, gels, microspheres, and aerosols. Typical routes of administering such pharmaceutical compositions include, without limitation, oral, topical, transdermal, inhalation, parenteral, sublingual, buccal, rectal, vaginal, and intranasal. The term parenteral as used herein includes subcutaneous injections, intravenous, intramuscular, intradermal, intrasternal injection or infusion techniques. Pharmaceutical compositions of the disclosure are formulated to allow the active ingredients contained therein to be bioavailable upon administration of the composition to a patient. Compositions that will be administered to a subject or patient take the form of one or more dosage units, where for example, a tablet may be a single dosage unit, and a container of a compound of the disclosure in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of a compound of the disclosure, or a pharmaceutically acceptable salt thereof, for treatment of a disease or condition of interest in accordance with the teachings of this disclosure.

A pharmaceutical composition of the disclosure may be in the form of a solid or liquid. In one aspect, the carrier(s) are particulate, so that the compositions are, for example, in tablet or powder form. The carrier(s) may be liquid, with the compositions being, for example, an oral syrup, injectable liquid, or an aerosol, which is useful in, for example, inhalatory administration.

When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer, or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion, or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer, and isotonic agent may be included.

The liquid pharmaceutical compositions of the disclosure, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose; agents to act as cryoprotectants such as sucrose or trehalose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition of the disclosure intended for either parenteral or oral administration should contain an amount of a compound of the disclosure such that a suitable dosage will be obtained.

The pharmaceutical composition of the disclosure may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device.

The pharmaceutical composition of the disclosure may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter, and polyethylene glycol.

The pharmaceutical composition of the disclosure may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the disclosure in solid or liquid form may include an agent that binds to the compound of the disclosure and thereby assists in the delivery of the compound. Suitable agents that may act in this capacity include a monoclonal or polyclonal antibody, or a protein.

The pharmaceutical composition of the disclosure may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the disclosure may be delivered in single phase, bi-phasic, or tri-phasic systems in order to deliver the active ingredient(s). Delivery of the aerosol includes the necessary container, activators, valves, sub-containers, and the like, which together may form a kit. One skilled in the art, without undue experimentation may determine preferred aerosols.

The pharmaceutical compositions of the disclosure may be prepared by methodology well known in the pharmaceutical art. For example, a pharmaceutical composition intended to be administered by injection can be prepared by combining the lipid nanoparticles of the disclosure with sterile, distilled water or other carrier to form a solution. A surfactant may be added to facilitate the formation of a homogeneous solution or suspension. Surfactants are compounds that non-covalently interact with the compound of the disclosure to facilitate dissolution or homogeneous suspension of the compound in the aqueous delivery system.

The compositions of the disclosure, or their pharmaceutically acceptable salts, are administered in a therapeutically effective amount, which will vary depending upon a variety of factors including the activity of the specific therapeutic agent employed; the metabolic stability and length of action of the therapeutic agent; the age, body weight, general health, sex, and diet of the patient; the mode and time of administration; the rate of excretion; the drug combination; the severity of the particular disorder or condition; and the subject undergoing therapy. Compositions of the disclosure may also be administered simultaneously with, prior to, or after administration of one or more other therapeutic agents. Such combination therapy includes administration of a single pharmaceutical dosage formulation of a composition of the disclosure and one or more additional active agents, as well as administration of the composition of the disclosure and each active agent in its own separate pharmaceutical dosage formulation. For example, a composition of the disclosure and the other active agent can be administered to the patient together in a single oral dosage composition such as a tablet or capsule, or each agent administered in separate oral dosage formulations. Where separate dosage formulations are used, the compounds of the disclosure and one or more additional active agents can be administered at essentially the same time, i.e., concurrently, or at separately staggered times, i.e., sequentially; combination therapy is understood to include all these regimens.

Preparation methods for the above compounds and compositions are described herein below and/or known in the art.

It will be appreciated by those skilled in the art that in the process described herein the functional groups of intermediate compounds may need to be protected by suitable protecting groups. Such functional groups include hydroxy, amino, mercapto, and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like.

Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T.W. and P.G.M. Wutz, Protective Groups in Organic Synthesis (1999), 3^{rd} Ed., Wiley. As one of skill in the art would appreciate, the protecting group may also be a polymer resin such as a Wang resin, Rink resin or a 2-chlorotrityl-chloride resin.

It will also be appreciated by those skilled in the art, although such protected derivatives of compounds of this disclosure may not possess pharmacological activity as such, they may be administered to a mammal and thereafter metabolized in the body to form compounds of the disclosure which are pharmacologically active. Such derivatives may therefore be described as “prodrugs.” All prodrugs of compounds of this disclosure are included within the scope of the disclosure.

Furthermore, all compounds of the disclosure which exist in free base or acid form can be converted to their pharmaceutically acceptable salts by treatment with the appropriate inorganic or organic base or acid by methods known to one skilled in the art. Salts of the compounds of the disclosure can be converted to their free base or acid form by standard techniques.

The following Reaction Scheme illustrates methods to make compounds of this disclosure, i.e., compounds of Formula (I):

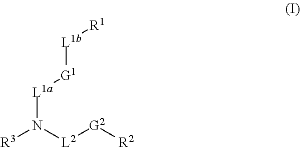

or a pharmaceutically acceptable salt, tautomer, or stereoisomer thereof, wherein G¹, G², R¹, R², R³, L¹, L^{1b}, and L² are as defined herein. It is understood that one skilled in the art may be able to make these compounds by similar methods or by combining other methods known to one skilled in the art. It is also understood that one skilled in the art would be able to make, in a similar manner as described below, other compounds of Formula (I) not specifically illustrated below by using the appropriate starting components and modifying the parameters of the synthesis as needed. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, e.g., Advanced Organic Chemistry: Reactions, Mechanisms, and Structure, 5th edition (Wiley, December 2000)) or prepared as described in this disclosure. Purification (e.g., silica gel chromatography, filtration, extraction, etc.) after each described reaction step is performed as needed.

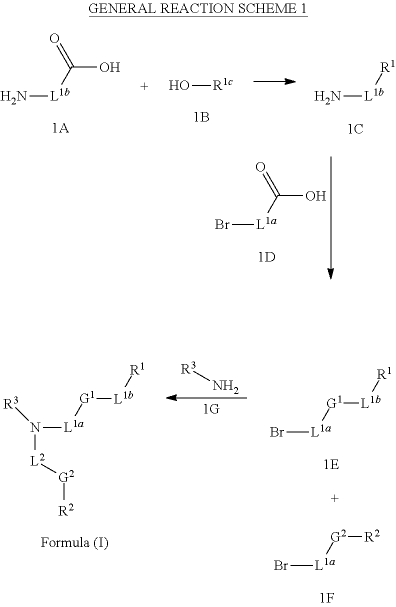

Embodiments of the compound of Formula (I) (e.g., Compound I-4) can be prepared according to General Reaction Scheme 1, wherein the variables (e.g., G¹, G², R¹, R², R³, L^{1a}, L^{1b}, L², and R^{1c}) are as defined herein.

Referring to General Reaction Scheme 1, reagents and starting materials (e.g., compounds 1A, 1B, 1D, and 1G) can be purchased from commercial sources or prepared according to methods familiar to one of ordinary skill in the art. A mixture of compound 1A and 1B is combined with suitable reagents and conditions to facilitate a coupling reaction (e.g., p-toluenesulfonic acid monohydrate in toluene at reflux). The resultant compound 1C can be combined with 1D with suitable reagents and reaction conditions (e.g., DMAP, NHS in DCM with DCC at room temperature) to afford compounds 1E and 1F. The desired products can then be reacted with compound 1G using appropriate conditions and reagents (e.g., DIPEA and heating) to afford a compound of Formula (I).

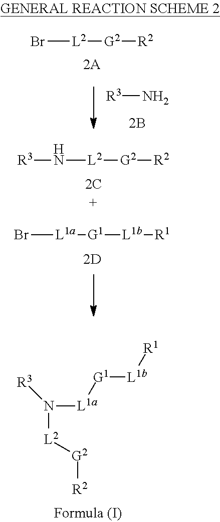

Embodiments of the compound of Formula (I) (e.g., Compound I-3) can be prepared according to General Reaction Scheme 2, wherein the variables (e.g., G¹, G², R¹, R², R³, L^{1a}, L^{1b}, L², and R^{1c}) are as defined herein.

Referring to General Reaction Scheme 2, reagents and starting materials (e.g., compounds 2A and 2B) can be purchased from commercial sources or prepared according to methods familiar to one of ordinary skill in the art. A mixture of compound 2A and compound 2B is combined with suitable reagents to facilitate a coupling reaction (e.g., K₂CO₃ in EtOH and heating). The desired product (compound 2C) can then be combined with compound 2D and subjected to suitable reaction conditions (e.g., DIPEA and heating) to afford a compound of Formula (I).

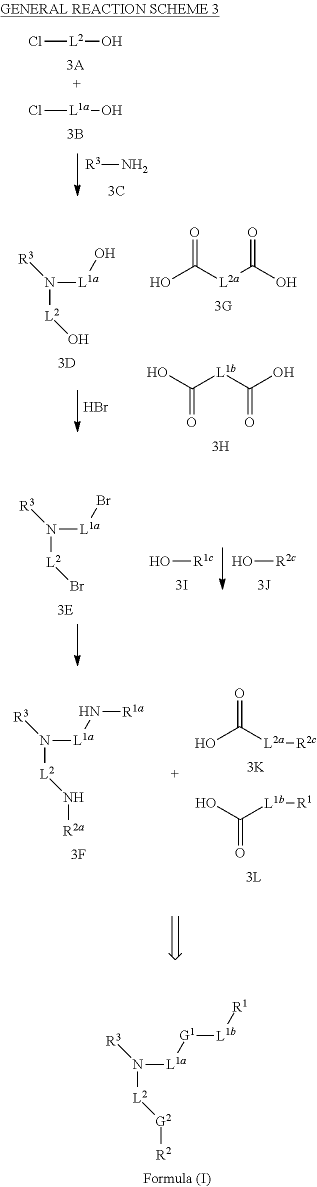

Embodiments of the compound of Formula (I) (e.g., Compound I-5) can be prepared according to General Reaction Scheme 3, wherein the variables (e.g., G¹, G², R¹, R², R³, L^{1a}, L^{1b}, L², R^{1c}, R^{2c}, and L^{2a}) are as defined herein.

Referring to General Reaction Scheme 2, reagents and starting materials (e.g., compounds 3A, 3B, 3C, 3G, 3H, 3I, and 3J) can be purchased from commercial sources or prepared according to methods familiar to one of ordinary skill in the art. Compounds 3A and 3B are combined with compound 3C using appropriate reagents and conditions (e.g., absolute ethanol, potassium carbonate, cesium carbonate, and sodium iodide and heating). The reaction product (compound 3D) can then be treated under appropriate conditions (e.g., hydrobromic acid and heating) to afford compound 3E, which can then be combined with H₂N—R¹a and H₂N—R^{2a} using suitable conditions (e.g., DIPEA, sodium iodide, and heating) to afford a compound 3F.

In parallel, compounds 3G and 3H are reacted with compounds 31 and 3J under appropriate conditions (e.g., DCC, triethylamine, DMAP). The resultant products (compounds 3K and 3L) can be reacted compound 3F to yield a compound of Formula (I).

It is understood that one skilled in the art may be able to make compounds of Formula (I) by similar methods or by combining other methods known to one skilled in the art. It is also understood that one skilled in the art would be able to make, in a similar manner as described herein, other compounds of Formula (I) not specifically illustrated below by using the appropriate starting components and modifying the parameters of the synthesis as needed. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, e.g., Advanced Organic Chemistry: Reactions, Mechanisms, and Structure, 5th edition (Wiley, December 2000)) or prepared as described in this disclosure.

### Example 1

### Luciferase mRNA In Vivo Evaluation Using

### Lipid Nanoparticle Compositions

A lipid of Formula (I), DSPC, cholesterol and PEG-lipid of Formula (II) are solubilized in ethanol at a molar ratio of 50:10:38.5:1.5 or 47.5:10:40.7:1.8. Lipid nanoparticles (LNP) are prepared at a total lipid to mRNA weight ratio of approximately 10:1 to 40:1. Briefly, the mRNA is diluted to 0.2 mg/mL in 10 to 50 mM citrate buffer, pH 4 to 6 or 10 to 25 mM acetate buffer, pH 4 to 6. Syringe pumps are used to mix the ethanolic lipid solution with the mRNA aqueous solution at a ratio of about 1:5 to 1:3 (vol/vol) with total flow rates above 15 mL/min. The ethanol is then removed, and the external buffer replaced with PBS by dialysis. Finally, the lipid nanoparticles are filtered through a 0.2 m pore sterile filter.

Studies are performed in 6-8-week-old female C57BL/6 mice (Charles River) or 8-10-week-old CD-1 mice (Charles River or Inotiv) according to guidelines established by an institutional animal care committee (ACC) and the Canadian Council on Animal Care (CCAC). Varying doses of mRNA-lipid nanoparticle are systemically administered by tail vein injection and animals euthanized at a specific time point (e.g., 4 hours) post-administration. Liver and spleen are collected in pre-weighed tubes, weights determined, immediately snap frozen in liquid nitrogen, and stored at −80° C. until processing for analysis.

For liver, approximately 50 mg is dissected for analyses in a 2 mL FastPrep tubes (MP Biomedicals, Solon OH). ¼″ ceramic sphere (MP Biomedicals) is added to each tube and 500-750 μL of Glo Lysis Buffer—GLB (Promega, Madison WI) equilibrated to room temperature is added to liver tissue. Liver tissues are homogenized with the FastPrep24 instrument (MP Biomedicals) at 2×6.0 m/s for 15 seconds. Homogenate is incubated at room temperature for 5 minutes prior to a 1:4 to 1:6 dilution in GLB and assessed using SteadyGlo Luciferase assay system (Promega). Specifically, 50 μL of diluted tissue homogenate is reacted with 50 μL of SteadyGlo substrate, shaken for 10 seconds followed by 5-minute incubation and then luminescence was quantitated using a CentroXS³ LB 960 luminometer (Berthold Technologies, Germany) or Filter Max F5 Microplate Reader (Molecular Devices, USA). The amount of protein assayed is determined by using the BCA protein assay kit (Pierce, Rockford, IL). Relative luminescence units (RLU) were then normalized to total μg protein or weight (g) of tissue assayed. To convert RLU to ng luciferase a standard curve is generated with QuantiLum Recombinant Luciferase (Promega).

The FLuc mRNA (7202) from Trilink Biotechnologies will express a luciferase protein, originally isolated from the firefly, photinus pyralis. FLuc is commonly used in mammalian cell culture to measure both gene expression and cell viability. It emits bioluminescence in the presence of the substrate, luciferin. This capped and polyadenylated mRNA modified with 5-methyoxyuridine and optimized for mammalian systems.

### Example 2

### Immunoglobulin G (Igg) mRNA In Vivo Evaluation Using Lipid Nanoparticle Compositions

A lipid of Formula (I), DSPC, cholesterol and PEG-lipid of Formula (II) are solubilized in ethanol at a molar ratio of 50:10:38.5:1.5 or 47.5:10:40.7:1.8. Lipid nanoparticles (LNP) are prepared at a total lipid to mRNA weight ratio of approximately 10:1 to 40:1. Briefly, the mRNA is diluted to 0.2 mg/mL in 10 to 50 mM citrate buffer, pH 4 to 6 or 10 to 25 mM acetate buffer, pH 4 to 6. Syringe pumps are used to mix the ethanolic lipid solution with the mRNA aqueous solution at a ratio of about 1:5 to 1:3 (vol/vol) with total flow rates above 15 mL/min. The ethanol is then removed, and the external buffer replaced with PBS by dialysis. Finally, the lipid nanoparticles are filtered through a 0.2 m pore sterile filter.

Studies are performed in 6-8-week-old CD-1/ICR mice (Charles River or Inotiv) according to guidelines established by an institutional animal care committee (ACC) and the Canadian Council on Animal Care (CCAC). Varying doses of mRNA-lipid nanoparticle are systemically administered by tail vein injection and animals euthanized at a specific time point (e.g., 24 hours) post-administration. The whole blood is collected, and the serum subsequentially separated by centrifuging the tubes of the whole blood at 2000×g for 10 minutes at 4° C. and stored at −80° C. until use for analysis.

For immunoglobulin G (IgG) ELISA (Life Diagnostics Human IgG ELISA kit), the serum samples are diluted at 100 to 20,000 folds with 1× diluent solution. 100 μL of diluted serum is dispensed into anti-human IgG coated 96-well plate in duplicate alongside human IgG standards and incubated in a plate shaker at 150 rpm at 25° C. for 45 minutes. The wells are washed 5 times with 1× wash solution using a plate washer (400 μL/well). 100 μL of HRP conjugate is added into each well and incubated in a plate shaker at the same condition above. The wells are washed 5 times again with 1× wash solution using a plate washer (400 μL/well). 100 μL of TMB reagent is added into each well and incubated in a plate shaker at the same condition above. The reaction is stopped by adding 100 μL of Stop solution to each well. The absorbance is read at 450 nm (A450) with a microplate reader. The amount of human IgG in mouse serum is determined by plotting A450 values for the assay standard against human IgG concentration.

### Example 3

### Determination of pKa of Formulated Lipids

As described elsewhere, the pKa of formulated lipids is correlated with the effectiveness of LNPs for delivery of nucleic acids (see Jayaraman et al, Angewandte Chemie, International Edition (2012), 51(34), 8529-8533; Semple et al, Nature Biotechnology 28, 172-176 (2010)). In some embodiments, the preferred range of pKa is ˜5 to ˜7. The pKₐ of each lipid may be determined in lipid nanoparticles using an assay based on fluorescence of 2-(p-toluidino)-6-napthalene sulfonic acid (TNS). Lipid nanoparticles comprising compound of Formula (I)/DSPC/cholesterol/PEG-lipid (50:10:38.5:1.5 or 47.5:10:40.7:1.8 mol %) in PBS at a concentration of 0.4 mM total lipid are prepared using the in-line process as described in Example 1. TNS is prepared as a 100 μM stock solution in distilled water. Vesicles are diluted to 24 μM lipid in 2 mL of buffered solutions containing 10 mM HEPES, 10 mM MES, 10 mM ammonium acetate, and 130 mM NaCl, where the pH ranged from 2.5 to 11. An aliquot of the TNS solution is added to give a final concentration of 1 μM and following vortex mixing fluorescence intensity is measured at room temperature in a SLM Aminco Series 2 Luminescence Spectrophotometer using excitation and emission wavelengths of 321 nm and 445 nm. A sigmoidal best fit analysis is applied to the fluorescence data and the pKₐ is measured as the pH giving rise to half-maximal fluorescence intensity.

### Example 4

### Determination of Efficacy of Lipid Nanoparticle Formulations Containing Various Cationic Lipids Using Igg mRNA Expression Rodent Model

Representative compounds of the disclosure shown in Table 2 were formulated using the following molar ratio: 50% cationic lipid/10% distearoylphosphatidylcholine (DSPC)/38.5% Cholesterol/1.5% PEG lipid 2-[2-(ω-methoxy(polyethyleneglycohooo)ethoxy]-N,N-ditetradecylacetamide) or 47.5% cationic lipid/10% DSPC/40.7% Cholesterol/1.8% PEG lipid. Relative activity was determined by measuring the amount of human IgG in mouse serum as described in Example 1. The activity was compared at a dose of 1.0 or 0.3 mg mRNA/kg and expressed as ng luciferase/g liver measured 4 hours after administration, as described in Example 1 or as μg IgG/mL serum measured 24 hours after administration, as described in Example 2. Compound numbers in Table 2 refer to the compound numbers of Table 1.

TABLE 2
Novel Cationic Lipids and Associated Activity

| | Liver Luc | Liver Luc | Serum IgG | Serum IgG |
|---|---|---|---|---|
| Compound | (ng/g) | (ng/g) | (μg/mL) | (μg/mL) |
| No. | @ 0.3 mg/kg | @ 1.0 mg/kg | @ 0.3 mg/kg | @ 1.0 mg/kg |
| I-2 | — | — | 70.7 ± 11.3 | 369.6 ± 59.4 |
| I-3 | 8191 ± 2369 | 25381 ± 4022 | — | — |
| I-5 | — | — | 57.5 ± 8.4 | — |
| I-11 | — | — | 35.6 ± 5.5 | — |
| I-12 | — | — | 29.6 ± 12.7 | — |
| I-13 | — | — | 11.3 ± 4.7 | — |
| I-14 | — | — | 33.4 ± 7.7 | — |
| I-15 | — | — | 48.2 ± 12.7 | 135.0 ± 37.2 |
| I-16 | — | — | 37.2 ± 9.7 | — |
| I-17 | — | — | 16.0 ± 2.6 | — |
| I-18 | — | — | 8.9 ± 4.9 | 32.4 ± 4.6 |
| I-19 | — | — | 12.3 ± 5.2 | — |
| I-20 | — | — | 23.6 ± 4.2 | 171.6 ± 50.4 |
| I-22 | — | — | 11.9 ± 5.9 | — |
| I-24 | — | — | 31.9 ± 6.4 | — |
| I-25 | — | — | 55.3 ± 24.8 | 192.4 ± 106.8 |
| I-27 | — | — | 15.6 ± 4.7 | — |
| I-28 | — | — | 13.2 ± 4.3 | — |
| I-32 | — | — | 70.8 ± 12.3 | — |
| I-33 | — | — | 75.8 ± 36.0 | — |
| I-34 | — | — | 5.0 ± 0.8 | — |
| I-37 | — | — | 63.0 ± 22.9 | — |
| I-38 | — | — | 21.9 ± 8.8 | — |
| I-40 | — | — | 13.8 ± 6.0 | — |
| I-41 | — | — | 19.2 ± 6.8 | — |
| I-43 | — | — | 49.9 ± 14.8 | — |
| I-45 | — | — | 18.3 ± 4.6 | — |
| I-46 | — | — | 34.4 ± 11.3 | — |
| I-48 | — | — | 27.5 ± 12.7 | — |
| I-50 | — | — | 11.6 ± 8.2 | — |
| I-52 | — | — | 13.0 ± 4.1 | - |

### Synthetic Example 1

### Synthesis of Bis(2-Hexyldecyl) 6,6′-((8,8′-(Methylazanediyl)Bis(Octanoyl))Bis(Azanediyl))Dihexanoate (Compound I-4)

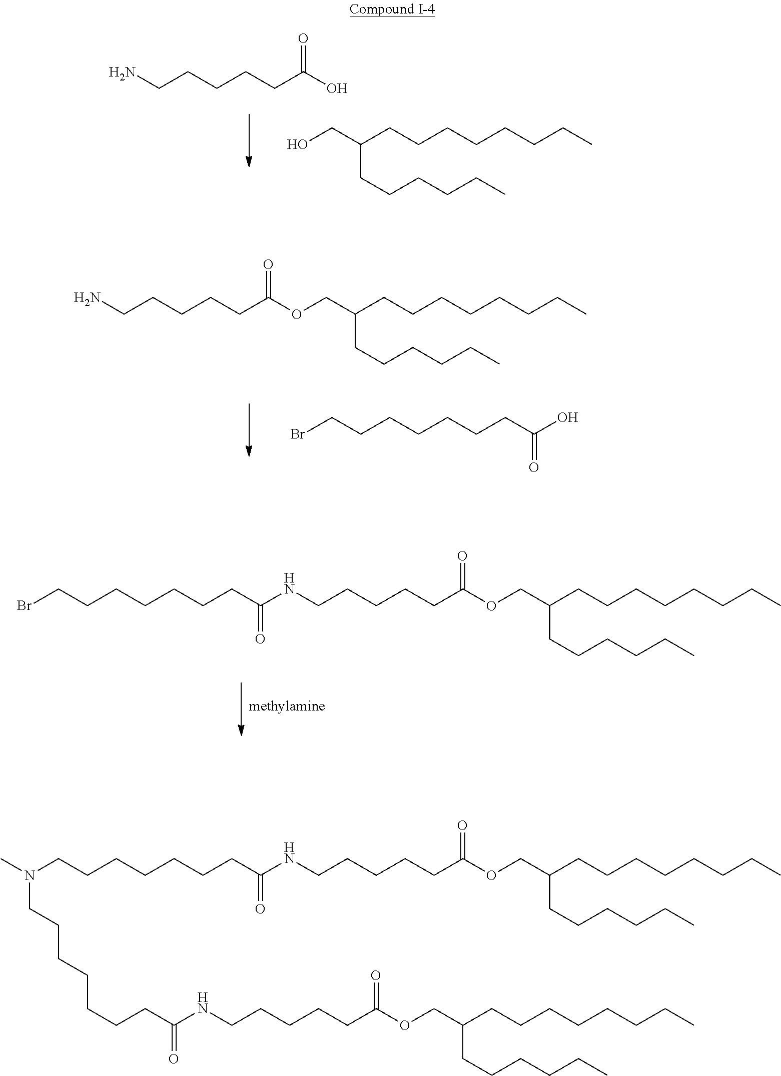

### Synthesis of 2-hexyldecyl 6-aminohexanoate

A mixture of 2-hexyl-1-decanol (1.25 eq, 25 mmol, 6.06 g), 6-Aminocaproic acid (2.62 g, 20 mmol), and p-toluene sulfonic acid monohydrate (1.1 eq, 22 mmol, 4.18 g) in toluene (70 mL) was heated to reflux for 16 hours under Dean-Stark conditions. The reaction mixture was cooled and basified with saturated NaHCO₃ solution. The two phases were separated. The aqueous phase was extracted with EtOAc. The organic phases were combined and concentrated to give a yellow oil (8.7 g). The crude product was purified flash dry column chromatography on silica gel. The column was first eluted with hexane/ethyl acetate (80:20) and then with chloroform/MeOH/NH₄OH (96:4:0.5 to 90:10:0.5). The desired product was obtained as a colorless oil (6.63 g, 18.65 mmol, 93%).

### Synthesis of 2-hexyldecyl 6-(8-bromooctanamido)hexanoate

To a mixture of 8-bromohexanoic acid (1.0 equiv, 1.0 mmol, 223 mg), and 4-dimethylaminopyridine (0.3 eq., 0.3 mmol, 36 mg) and N-hydroxysuccinimide (1.0 equiv, 1.0 mmol, 115 mg) in 5 mL of CH₂Cl₂ was added DCC (1.05 equiv, 1.05 mmol, 216 mg) and the mixture stirred at room temperature for about 2.5 h. The reaction mixture was filtered into a flask containing 2-hexyldecyl 6-aminohexanoate (1 equiv, 1 mmol, 356 mg). The resulting solution was stirred at room temperature (RT) overnight. After concentration of the reaction mixture, a white solid was obtained. The solid was taken up in dichloromethane (DCM; 25 mL) and was loaded on a short silica gel column under reduced pressure. The column was eluted with a mixture of DCM and methanol (MeOH; 100:0 to 99:1) under reduced pressure. The desired product was obtained as a white solid (454 mg, 0.81 mmol, 81%).

### Synthesis of bis(2-hexyldecyl) 6,6′-((8,8′-(methylazanediyl)bis(octanoyl))bis(azanediyl))dihexanoate (Compound I-4)

A mixture of 2-hexyldecyl 6-(8-bromooctanamido)hexanoate (0.454 g, 0.81 mmol), anhydrous acetonitrile (15 mL) and N,N-diisopropylethylamine (0.344 mL) and methylamine (0.07 mL, 33 wt. % in absolute ethanol) in a pressure flask was heated at 74° C. (oil bath) for 16 hours. Then the reaction mixture was concentrated. The residue was taken up in a mixture of hexane, ethyl acetate, MeOH and triethylamine (Et₃N; 80:20:3:1) and was filtered through a pad of silica gel. The pad was washed with the same solvent mixture. Concentration of the filtrate and washings gave a pale paste. The crude product was further purified by flash dry column chromatography on silica gel using 0 to 5% MeOH in chloroform with a trace of Et₃N as eluting solvent mixture. The desired product was obtained as a colorless oil (85 mg, 0.086 mmol, 21%).

¹H NMR (400 MHz, CDCl₃, at 7.26 ppm) δ: 5.50 (s, 2H), 3.96 (d, 5.8 Hz, 4H), 3.24 (q, 6.7 Hz, 4H), 2.33-2.25 (m, 8H), 2.18 (s, 3H), 2.14 (t, 7.5 Hz, 4H), 1.68-1.57 (m, 10H), 1.54-1.39 (m, 8H), 1.39-1.16 (m, 64H), 0.88 (t, 6.5 Hz, 12H). ESI-MS: MW for C₆₁H₁₁₉N₃O₆ [M+H]⁺ Calc. 990.9; Found 991.3.

### Synthetic Example 2

### Synthesis of 2-Hexyldecyl 6-(8-((8-(Didecylamino)-8-Oxooctyl)(Methyl)Amino)Octanamido)Hexanoate (Compound I-3)

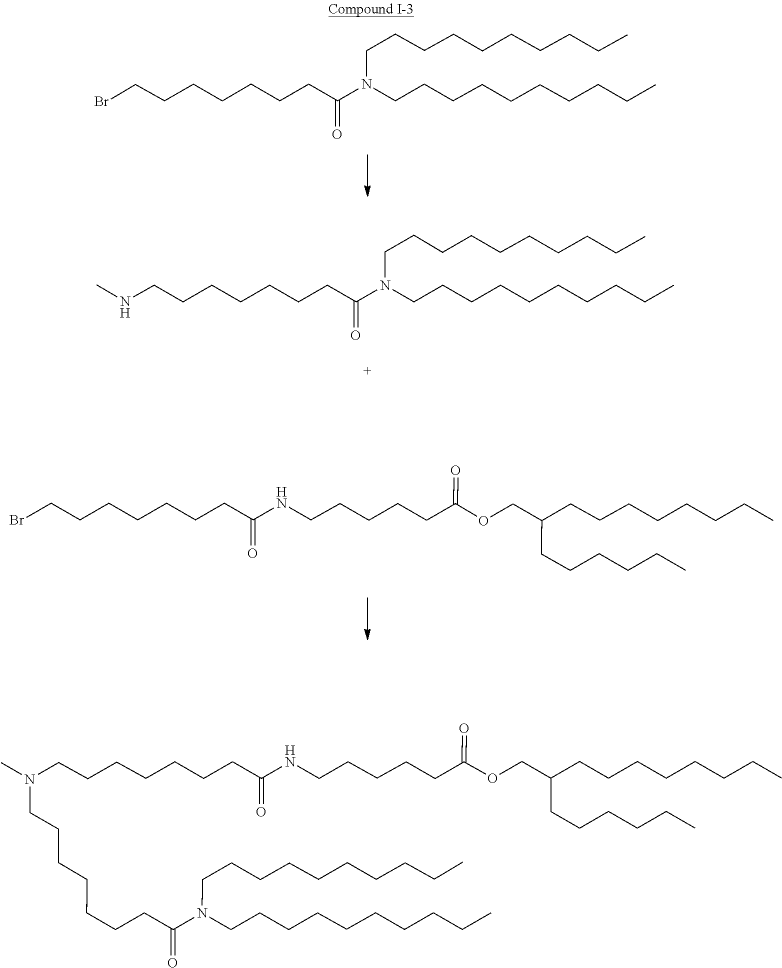

### Synthesis of N,N-didecyl-8-(methylamino)octanamide

A mixture of methylamine (7.5 mL, 33 wt. % in absolute ethanol), 8-bromo-N,N-didecyloctanamide (1.506 g, 3 mmol; made from 8-bromooctanoic acid and didecylamine) and K₂CO₃ (3 mmol, 414 mg) in 30 mL of EtOH was sealed in a pressure bottle and heated at 75° C. (oil bath) for 16 hours. The reaction mixture was diluted with DCM and filtered through a pad of diatomaceous earth (e.g., Celite®). The solid was washed with more DCM. The filtrate was concentrated to dryness, giving the desired product (yellow oil, 1.35 g). The product was used for the next step without further purification.

### Synthesis of 2-hexyldecyl 6-(8-((8-(didecylamino)-8-oxooctyl) (methyl)amino)octanamido) hexanoate (Compound I-3)

A mixture of N,N-didecyl-8-(methylamino)octanamide (0.55 mmol, 250 mg), 2-hexyldecyl 6-(8-bromooctanamido)hexanoate (0.55 mmol, 310 mg), anhydrous acetonitrile (15 mL) and N,N-diisopropylethylamine (0.3 mL) was heated for 16 hours in a sealed pressure flask at 74° C. (oil bath). Then the reaction mixture was concentrated. The residue was taken up in a mixture of hexane, ethyl acetate, MeOH and Et₃N (80:20:3:1) and was filtered through a pad of silica gel. The pad was washed with the same solvent mixture. Concentration of the filtrate gave a colorless oil. The crude product was further purified by flash dry column chromatography on silica gel using 0 to 5% MeOH in chloroform with a trace of Et₃N as eluting solvent mixture. The desired product was obtained as a colorless oil (37 mg).

¹H NMR (400 MHz, CDCl₃ at 7.26 ppm) δ: 5.55-5.50 (m, 1H), 3.96 (d, 5.8 Hz, 2H), 3.51 (t, 5.0 Hz, 2H), 3.30-3.16 (m, 6H), 2.33-2.24 (m, 8H), 2.19 (t, 3H), 2.14 (t, 7.6 Hz, 2H), 1.68-1.57 (m, overlaps with water signal, estimated 7H), 1.57-1.40 (m, 10H), 1.40-1.15 (m, 66H), 0.91-0.85 (m, 12H). ESI-MS: MW for C₅₉H₁₁₇N₃O₄ [M+H]⁺ Calc. 932.9; Found 933.0.

### Synthetic Example 3

### Synthesis of Bis(2-Hexyldecyl) 8,8′-((10,10′-(Methylazanediyl)Bis(Decanoyl))Bis(Azanediyl))Dioctanoate (Compound I-2)

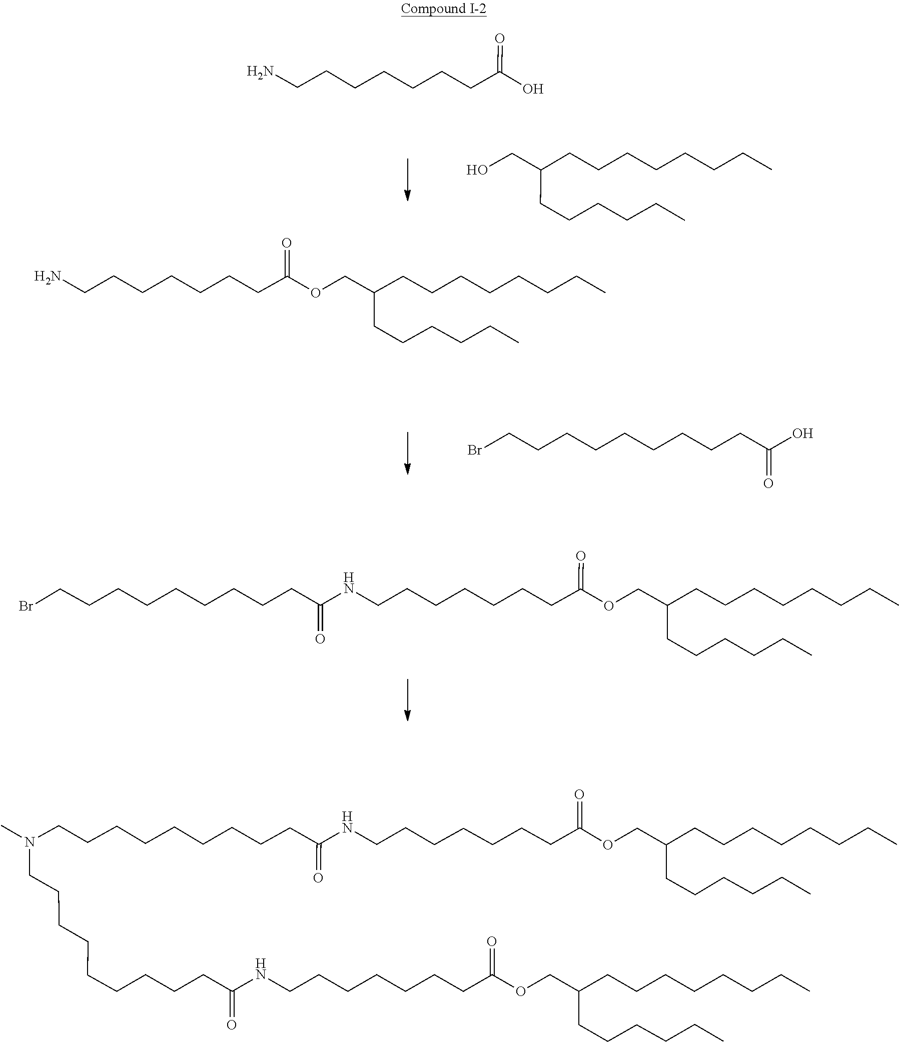

### Synthesis of 2-hexyldecyl 8-aminooctanoate

2-hexyldecyl 8-aminooctanoate was prepared in a similar manner to 2-hexyldecyl 6-aminohexanoate from 2-hexyl-1-decanol (1.25 eq, 5 mmol, 1.21 g) and 8-Aminooctanoic acid (4 mmol, 636 mg) to yield 1.467 g (3.82 mmol, 95%) of yellow oil.

### Synthesis of 2-hexyldecyl 8-(10-bromodecanamido)octanoate

2-hexyldecyl 8-(10-bromodecanamido)octanoate was prepared according to the general procedures of Synthetic Example 1, from 10-bromodectanoic acid (1.0 eq, 1.0 mmol, 251 mg) and 2-hexyldecyl 8-aminooctanoate (1.0 mmol, 384 mg) to yield 501 mg (0.81 mmol, 81%) of white solid.

### Synthesis of bis(2-hexyldecyl) 8,8′-((10,10′-(methylazanediyl)bis(decanoyl)) bis(azanediyl)) dioctanoate (Compound I-2)

Compound I-2 was prepared according to the general procedures of Synthetic Example 1, to yield 162 mg (0.15 mmol, 36%) of colorless oil.

¹H NMR (400 MHz, CDCl₃ at 7.26 ppm) δ: 5.45-5.39 (m, 2H), 3.96 (d, 5.8 Hz, 4H), 3.23 (q, 6.7 Hz, 4H), 2.29 (t, 7.4 Hz, 8H), 2.191 (s, 3H), 2.14 (t, 7.5 Hz, 4H), 1.68-1.57 (m, 10H), 1.54-1.39 (m, 8H), 1.39-1.19 (m, 80H), 0.88 (t, 6.5 Hz, 12H). ESI-MS: MW for C₆₉H₁₃₅N₃O₆ [M+H]⁺ Calc. 1103.0; Found 1103.1.

### Synthetic Example 4

### Synthesis of Bis(2-Hexyldecyl) 8,8′-((10,10′-((2-Hydroxyethyl)Azanediyl)Bis(Decanoyl))Bis(Azanediyl))Dioctanoate (Compound I-1)

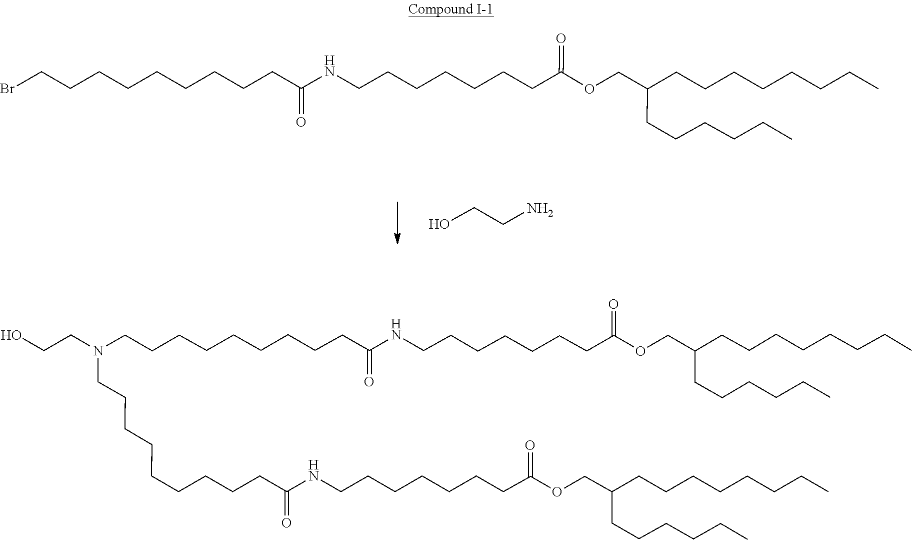

### Synthesis of bis(2-hexyldecyl) 8,8′-((10,10′-((2-hydroxyethyl)azanediyl)bis(decanoyl)) bis(azanediyl)) dioctanoate (Compound I-1)

Compound I-1 was prepared according to the general procedures of Synthetic Example 1, to yield 109 mg (0.096 mmol, 24%) of colorless oil.

¹H NMR (400 MHz, CDCl₃ at 7.26 ppm) δ: 5.50 (t, 5 Hz, 2H), 3.96 (d, 5.8 Hz, 4H), 3.58-3.47 (br. 2H), 2.23 (q, 6.7 Hz, 4H), 2.62-2.53 (br. 2H), 2.49-2.39 (br. 4H), 2.29 (t, 7.5 Hz, 4H), 2.14 (t, 7.5 Hz, 4H), 1.68-1.56 (m, 10H), 1.54-1.39 (m, 8H), 1.37-1.19 (m, 80H), 0.88 (t, 6.6 Hz, 12H). ESI-MS: MW for C₇₀H₁₃₇N₃O₇ [M+H]⁺ Calc. 1133.1; Found 1133.3.

### Synthetic Example 5

### Synthesis of Bis(2-Hexyldecyl) 9,9′-(((Methylazanediyl)Bis(Hexane-6,1-Diyl))Bis(Hexylazanediyl))Bis(9-Oxononanoate) (Compound 1-5)

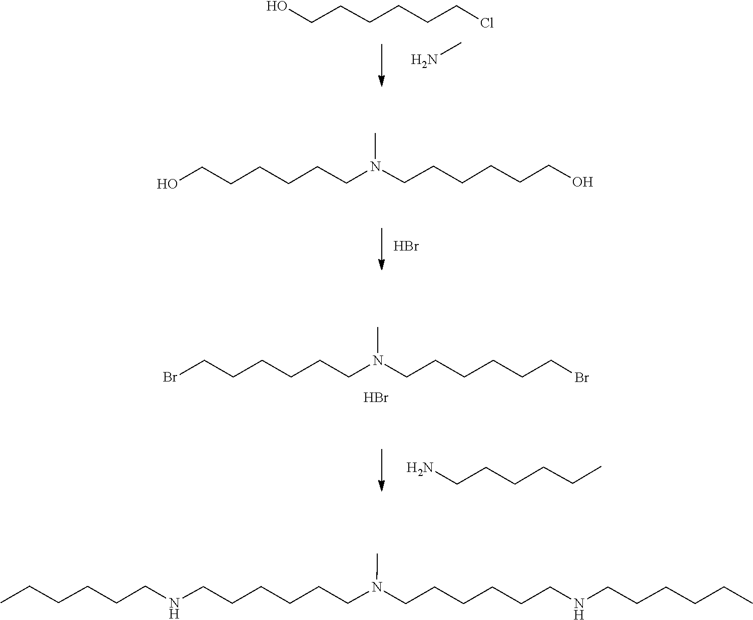

### Synthesis of 6,6′-(methylazanediyl)bis(hexan-1-ol)

To a mixture of 6-chloro-1-hexanol (22.8 mmol, 3.11 g, 3.04 mL), absolute ethanol (60 mL), potassium carbonate (2 eq, 24 mmol, 3.32 g), cesium carbonate (eq, 500 mg) and sodium iodide (40 mg) was added methylamine (2 mmol, 1.49 mL, 33 wt. % in absolute ethanol). The mixture was heated for 16 hours in a sealed pressure flask (oil bath: 68° C.). The reaction was monitored by TLC. More methylamine (0.1 mL), sodium iodide (300 mg) and potassium carbonate (1 g) were added to the reaction mixture. Heating was resumed. After an additional 4 days, more 6-chloro-1-hexanol (1.5 mL) and sodium iodide (220 mg) were added. Heating was continued at 76° C. for an additional 3 days. Finally, the mixture was cooled and filtered. The filtrate was concentrated, and the residue was taken up in DCM and filtered. The filtrate was concentrated to dryness under reduced pressure. This gave 4.86 g brown viscous oil. The oil was taken up in DCM (100 mL) and loaded on a short column of silica gel under reduced pressure. The column was eluted with a gradient mixture of DCM, methanol, and concentrated ammonia solution (100:0:0 to 85:15:1). Fractions containing the desired product were combined and concentrated. The residue was taken up in DCM and filtered. The filtrate was concentrated to dryness to give the desired product as brown viscous oil (1.40 g, 6.05 mmol, 50%). The product was used for the next step without further purification.

### Synthesis of 6-bromo-N-(6-bromohexyl)-N-methylhexan-1-amine

Hydrobromic acid (6 mL, 48 wt. % solution in water) was added slowly (over 5 minutes while stirring) to 6,6′-(methylazanediyl)bis(hexan-1-ol) (1.40 g, 6.05 mmol). The reaction mixture was then heated at 105° C. (oil bath) for 2 hours. Then, the reaction mixture was cooled a little and toluene (50 mL) was slowly added. The reaction was heated to reflux, and water was removed azeotropically. The reaction mixture was cooled to room temperature and then transferred to a pressure flask and concentrated to dryness under reduced pressure (brown oil 2.62 g, 5.97 mmol, 98%). TLC (CHCl₃/MeOH=9:1) showed a major spot. The product was used for the next step without further purification.

### Synthesis of N¹-hexyl-N⁶-(6-(hexylamino)hexyl)-N⁶-methylhexane-1,6-diamine

A mixture of 6-bromo-N-(6-bromohexyl)-N-methylhexan-1-amine (2.62 g, 5.97 mmol), hexylamine (10 eq. 120 mmol, 12.14 g), N,N-diisopropylethylamine (6.0 mmol, 1.04 mL), sodium iodide (20 mg) and acetonitrile (30 mL) in a pressure flask was sealed and heated at 76° C. (oil bath) for 24 h. The mixture was then concentrated at about 75° C. (about 9 mmHg) to remove solvent and the excess hexylamine. The residue (yellow oil/solid) was taken up in DCM and filtered. The filtrate was concentrated under reduced pressure, giving a yellow foam. The residue was taken up in NaOH solution (1.44 g of sodium hydroxide in 10 mL of water) and then concentrated at 75° C. for 1.5 hours under reduced pressure. The residue was taken up DCM and filtered. The filtrate was concentrated under reduced pressure to dryness, giving a pale paste (2.671 g). The product was used for the next step without further purification.

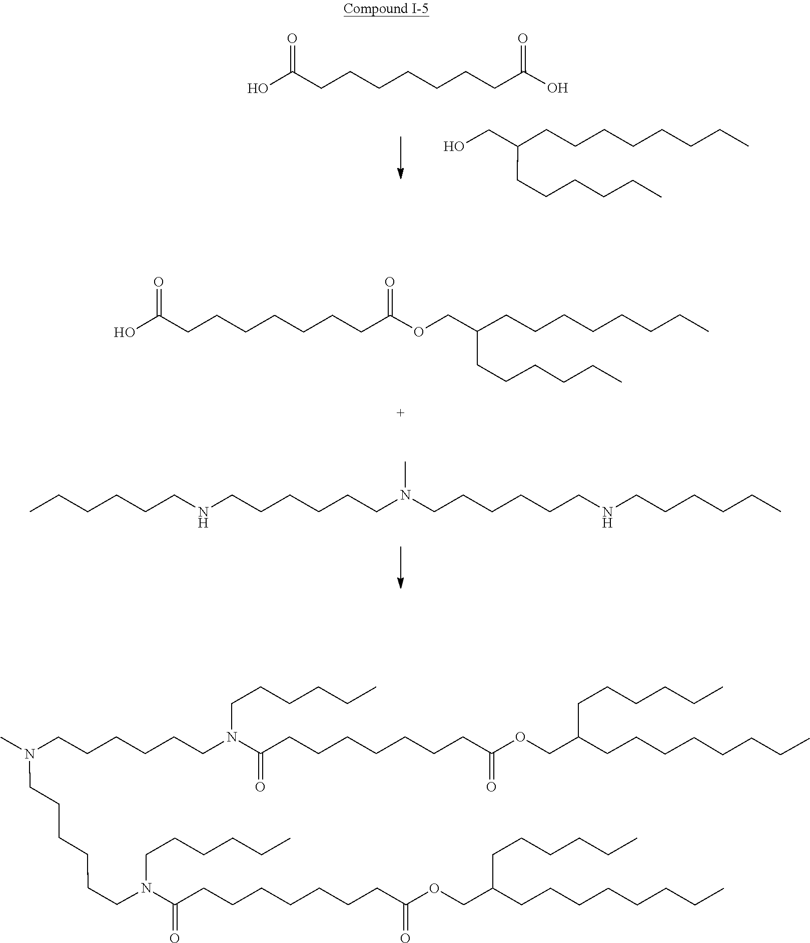

### Synthesis of 9-((2-hexyldecyl)oxy)-9-oxononanoic acid

DCC (1.3 equiv. 2.38 g) was added to a solution of 2-hexyl-1-decanol (1 equiv. 8.85 mmol, 2.15 g), azelaic acid (1.2 eq., 2 g, 10.6 mmol), triethylamine (2 eq, 17.7 mmol, 2.47 mL) and 4-dimethylaminopyridine (DMAP, 325 mg) in dichloromethane (40 mL). After the resulting mixture was stirred at room temperature for 2 days, The reaction mixture and filtered. The filtrate was concentrated. The residue was taken up in hexane (100 mL) and filtered through a pad of silica gel (dry column 6.5 cm w×4 cm h, 230-400 mesh silica gel). The column was eluted with a gradient mixture of hexane and ethyl acetate (100:0:0 to 75:25). Fractions containing the desired product were combined and concentrated (2.095 g, colorless oil, 5.08 mmol, 57%). The product was used for the next step without further purification.

### Synthesis of bis(2-hexyldecyl) 9,9′-(((methylazanediyl)bis(hexane-6,1-diyl))bis(hexylazanediyl))bis (9-oxononanoate) (Compound I-5)

To a solution of 9-((2-hexyldecyl)oxy)-9-oxononanoic acid (1.51 mmol, 0.623 g) in DCM (15 mL) and dimethylformamide (DMF; 1 drop) was added dropwise oxalyl chloride (2 eq, 2.34 mmol, 191 mg, 0.13 mL) at room temperature. The resulting mixture was stirred at room temperature overnight. The mixture was concentrated under reduced pressure at 35° C. The residue was dissolved in 8 mL of DCM and added to a solution of Ni-hexyl-N⁶-(6-(hexylamino)hexyl)-N⁶-methylhexane-1,6-diamine (300 mg, 0.75 mmol) and triethylamine (0.3 mL) and DMAP (5 mg) in DCM (5 mL) at room temperature. After addition, the mixture was stirred at room temperature for 3 hours. The reaction mixture was diluted with a mixture of hexane and ethyl acetate and triethylamine (80:20:1) and was filtered through a pad of silica gel. The pad was washed with the same solvent mixture. Concentration of the filtrate led to a yellow oil (256 mg). The crude product was further purified by flash dry column chromatography on silica gel using 0 to 5% MeOH in chloroform with a trace of Et₃N as eluting solvent mixture. The desired product was obtained as a colorless oil (232 mg, 0.20 mmol, 26%).

¹H NMR (400 MHz, CDCl₃ at 7.26 ppm) δ: 3.96 (d, 5.8 Hz, 4H), 3.30-3.25 (m, 4H), 3.22-3.16 (m, 4H), 2.33-2.23 (m, 12H), 2.19 (t, 4.0 Hz, 3H), 1.67-1.58 (m, 10H), 1.56-1.38 (m, 12H), 1.38-1.18 (m, 80H), 0.93-0.85 (m, 18H). ESI-MS: MW for C₇₅H₁₄₇N₃O₆ [M+H]⁺ Calc. 1187.1; Found 1187.3.

### Synthetic Example 6

### Bis(2-Hexyldecyl) 6,6′-((8,8′-(Methylazanediyl)Bis(Octanoyl))Bis(Methylazanediyl))Dihexanoate (Compound I-30)

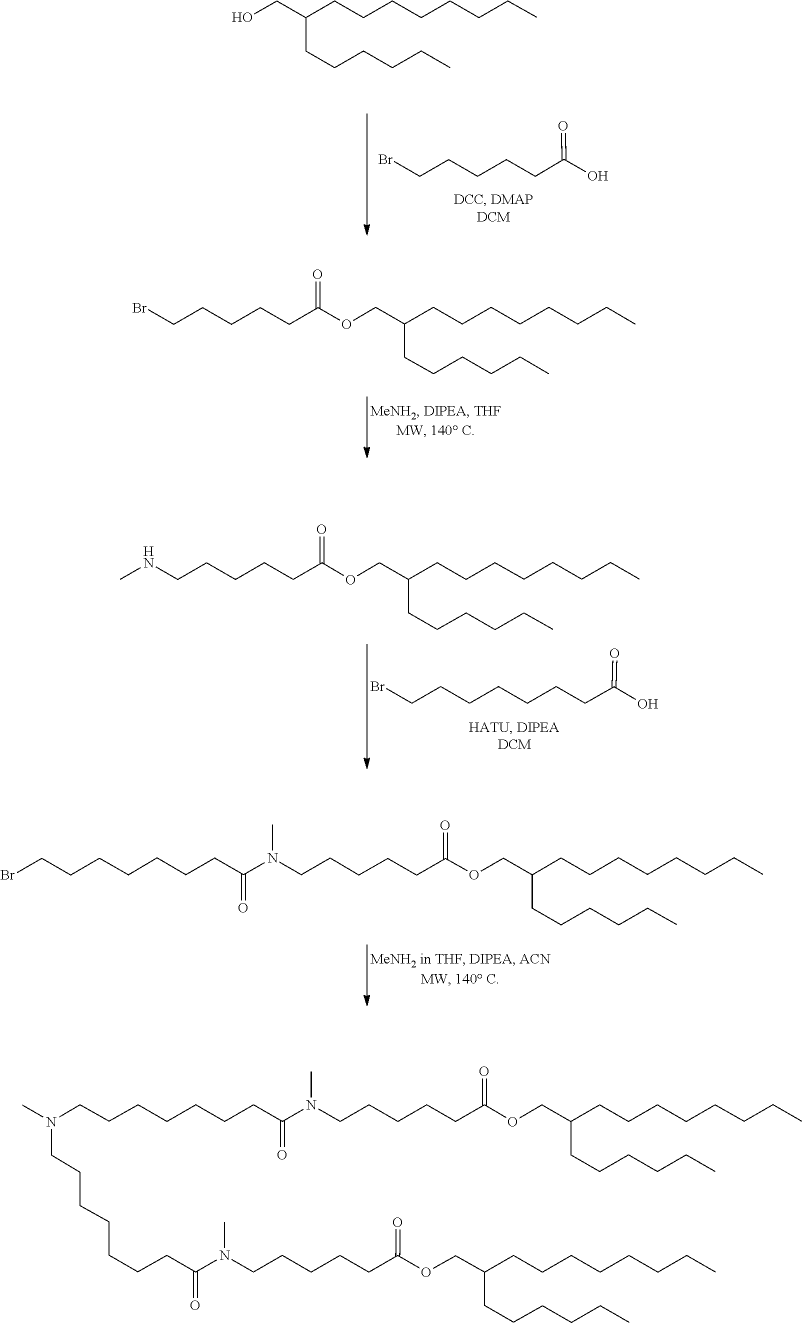

### Synthesis of 2-hexyldecyl 6-bromohexanoate

To a solution of 2-hexyl-1-decanol (57.9 mmol, 14.0 g), DMAP (10.5 mmol, 1.29 g) and 6-bromohexanoic acid (52.6 mmol, 10.3 g) in DCM (170 mL) was added DCC (68.4 mmol, 14.1 g) dissolved in 50 ml DCM. The reaction mixture was stirred at room temperature over night. Reaction mixture was diluted with hexanes (200 ml), filtered, concentrated, and purified via flash chromatography (0% to 15% EtOAc in Hexanes) to give 2-hexyldecyl 6-bromohexanoate (18.1 g, 82%). ESI-MS: m/z calculated for C₂₂H₄₃BrO₂=418.2, found [M+H]⁺=419.4.

### Synthesis of 2-hexyldecyl 6-(methylamino)hexanoate

To 2-hexyldecyl 6-bromohexanoate (1.3 mmol, 560 mg), 2M THF solution of methylamine (13.3 mmol, 6.67 mL) and DIPEA (2.67 mmol, 345 mg, 466 μL) were added. The reaction mixture was heated via microwave at 140° C. for 30 min and then concentrated under vacuum. The residue was partitioned between water and EtOAc, dried over anhydrous sodium sulfate, filtered, and concentrated again. The crude 2-hexyldecyl 6-(methylamino)hexanoate (493 mg, 99%; 78% purity by UPLC-CAD/MS) was used in the next step without any further purification. ESI-MS: m/z calculated for C₂₃H₄₇NO₂=369.4, found [M+H]⁺=370.5.

### Synthesis of 2-hexyldecyl 6-(8-bromo-N-methyloctanamido)hexanoate

To a solution of 2-hexyldecyl 6-(methylamino)hexanoate (78% purity; ca. 1.0 mmol, 493 mg), 8-bromooctanoic acid (1.6 mmol, 357 mg) and DIPEA (2.67 mmol, 345 mg, 466 μL) in DCM (10 mL), HATU (1.74 mmol, 660 mg) was added in one portion. The reaction mixture was stirred for 30 minutes and then washed with saturated NaHCO₃. The organic layer was separated, dried over anhydrous Na₂SO₄ and concentrated. Purification via flash chromatography (0% to 50% EtOAc in Hexanes spiked with 1% Et₃N) gave 2-hexyldecyl 6-(8-bromo-N-methyloctanamido)hexanoate (480 mg, ca. 83%). ESI-MS: m/z calculated for C₃₁H₆₀BrNO₃=573.4, found [M+H]⁺=574.6.

### Synthesis of Compound I-30

A mixture of 2-hexyldecyl 6-(8-bromo-N-methyloctanamido)hexanoate (835 μmol, 480 mg), 2M methylamine solution in THE (501 μmol, 251 μL), and DIPEA (1.67 mmol, 216 mg, 292 μL) in ACN (1.0 mL) was heated via microwave at 140° C. for 30 min and then concentrated under vacuum. The residue was partitioned between water and EtOAc, dried over anhydrous sodium sulfate, filtered, and concentrated again. The crude product was purified via flash chromatography (0% to 100% EtOAc in Hexanes spiked with 1% Et₃N) to give desired product (201 mg, 47%).

¹H NMR (400 MHz, CDCl₃) δ 3.96 (s, 4H), 3.41-3.29 (m, 2.2H), 3.29-3.19 (m, 1.8H), 2.96 (s, 3.3H), 2.90 (s, 2.7H), 2.40-2.22 (m, 12H), 2.19 (s, 3H), 1.80-1.38 (m, 23H), 1.38-1.19 (m, 66H), 0.88 (t, J=6.6 Hz, 12H). ESI-MS: m/z calculated for C₆₃H₁₂₃N₃O₆=1017.9, found [M+H]⁺=1019.2.

### Synthetic Example 7

### Bis(2-Hexyldecyl) 6,6′-((8,8′-(Methylazanediyl)Bis(Octanoyl))Bis(Isopropylazanediyl))Dihexanoate (Compound I-28)

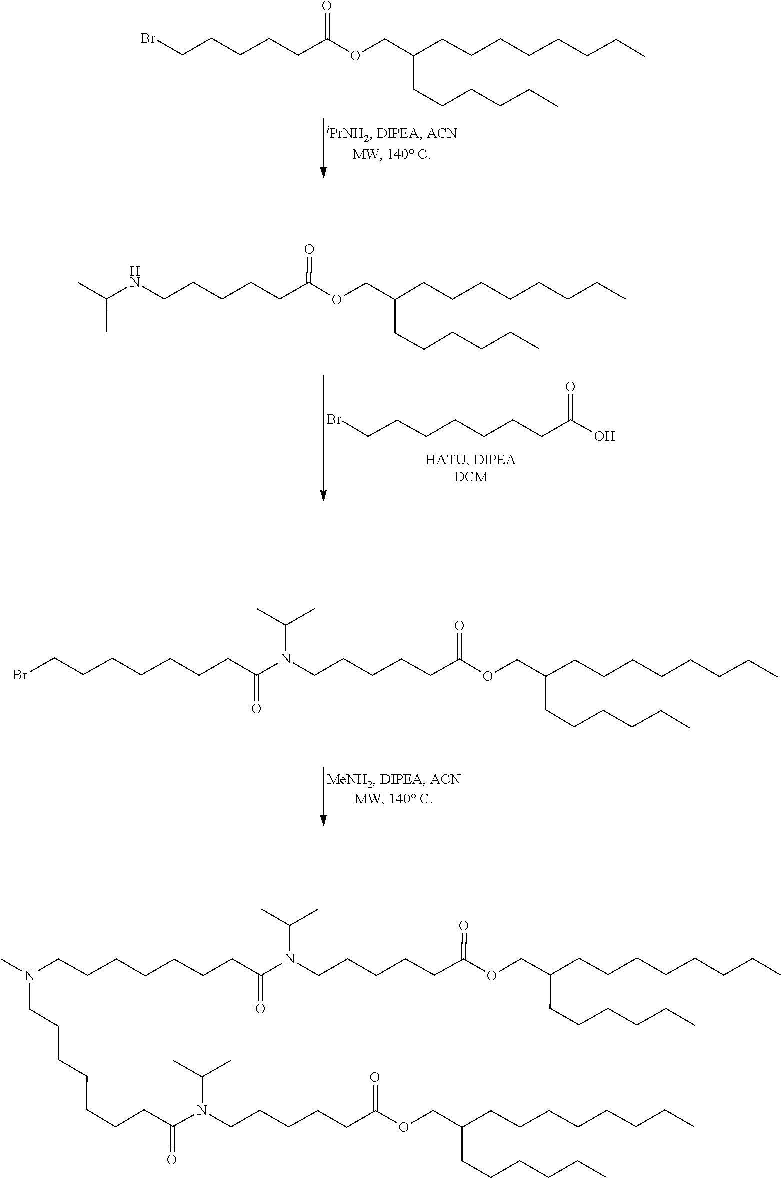

### Synthesis of 2-hexyldecyl 6-(isopropylamino)hexanoate

A mixture of 2-hexyldecyl 6-bromohexanoate (2.36 mmol, 990 mg), isopropyl amine (7.08 mmol, 610 mg) and DIPEA (4.72 mmol, 472 mg, 824 μL) in ACN (14.2 mL) was heated via microwave at 140° C. for 30 min and then concentrated under vacuum. The residue was partitioned between water and EtOAc, dried over anhydrous sodium sulfate, filtered, and concentrated again.

The crude product was purified via flash chromatography (0% to 10% MeOH in DCM) to give 2-hexyldecyl 6-(isopropylamino)hexanoate (900 mg, 96%). ESI-MS: m/z calculated for C₂₅H₅₁NO₂=397.4, found [M+H]⁺=398.6.

### Synthesis of 2-hexyldecyl 6-(8-bromo-N-isopropyloctanamido)hexanoate

2-Hexyldecyl 6-(8-bromo-N-isopropyloctanamido)hexanoate was prepared from (448 μmol, 178.2 mg), 8-bromooctanoic acid (538 μmol, 120 mg), DIPEA (896 μmol, 116 mg, 157 μL) and HATU (583 μmol, 221 mg) by following the procedures outlined herein. Yield (270 mg, 99%). ESI-MS: m/z calculated for C₃₃H₆₄BrNO₃=601.4, found [M+H]⁺=602.5.

### Synthesis of Compound I-28

Compound I-28 was prepared from starting material as shown (448 μmol, 270 mg), methyl amine (134 μmol, 2M THE solution, 134 μL) and DIPEA (896 μmol, 116 mg, 156 μL) in ACN (0.4 mL) following the procedures outlined herein. Yield (95 mg, 39%).

¹H NMR (400 MHz, CDCl₃) δ 4.65 (p, J=6.8 Hz, 1H), 4.03 (p, J=6.7 Hz, 1H), 4.00-3.90 (m, 4H), 3.17-3.03 (m, 4H), 2.41-2.20 (m, 12H), 2.18 (s, 3H), 1.78-1.50 (m, 17H), 1.50-1.40 (m, 4H), 1.39-1.21 (m, 65H), 1.17 (d, J=6.7 Hz, 6H), 1.12 (d, J=6.8 Hz, 6H), 0.88 (t, J=6.7 Hz, 12H). ESI-MS: m/z calculated for C₆₇H₁₃₁N₃O₆=1074.0, found [M+H]⁺=1075.26.

### Synthetic Example 8

### Bis(2-Hexyldecyl) 6,6′-((6,6′-(Methylazanediyl)Bis(Hexanoyl))Bis(Isopropylazanediyl))Dihexanoate (Compound I-26)

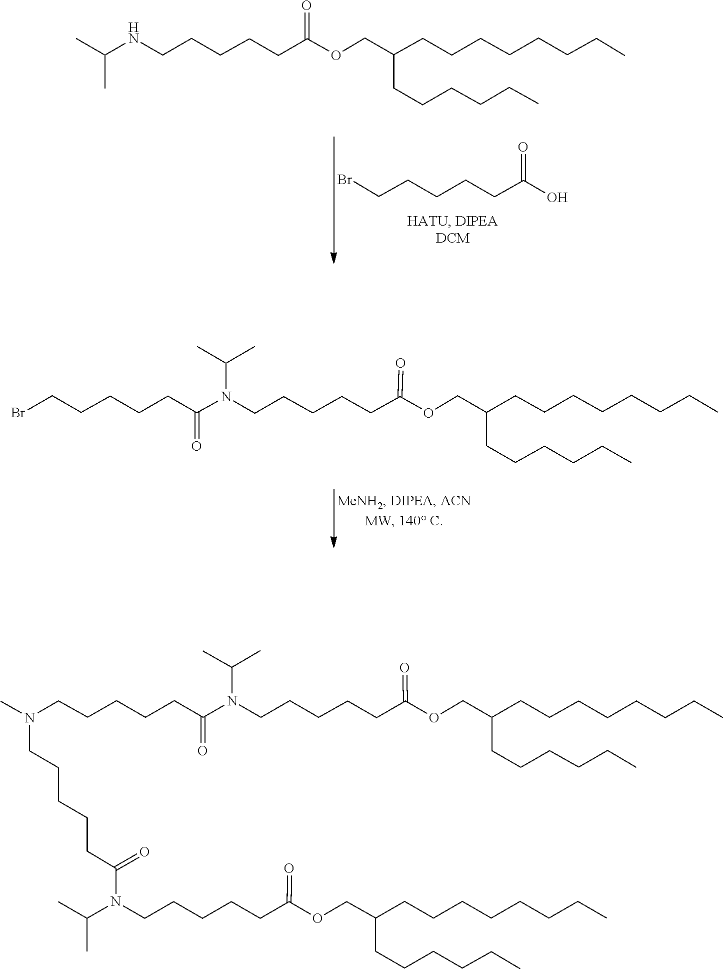

### Synthesis of 2-hexyldecyl 6-(6-bromo-N-isopropylhexanamido)hexanoate

2-Hexyldecyl 6-(6-bromo-N-isopropylhexanamido)hexanoate was prepared from starting material as shown (541 μmol, 215 mg), 8-bromohexanoic acid (649 μmol, 127 mg), DIPEA (1.08 mmol, 140 mg, 189 μL) and HATU (703 μmol, 267 mg) by following the procedures outlined herein. Yield (280 mg, 90%). ESI-MS: m/z calculated for C₃₁H₆₀BrNO₃=573.4, found [M+H]+=574.5.

### Synthesis of Compound I-26

Compound I-26 was prepared from starting material as shown (468 μmol, 269 mg), methyl amine (281 μmol, 2M THF solution, 140 μL) and DIPEA (935 μmol, 171 mg, 163 μL) in ACN (0.4 mL) following the procedures outlined herein. Yield (99 mg, 42%).

¹H NMR (400 MHz, CDCl₃) δ 4.70-4.58 (m, 1H), 4.10-4.01 (m, 1H), 4.00-3.90 (m, 4H), 3.17-3.03 (m, 4H), 2.43-2.23 (m, 12H), 2.20 (s, 3H), 1.75-1.42 (m, 23H), 1.41-1.22 (m, 57H), 1.17 (d, J=6.7 Hz, 6H), 1.12 (d, J=6.8 Hz, 6H), 0.88 (t, J=6.7 Hz, 12H). ESI-MS: m/z calculated for C₆₃H₁₂₃N₃O₆=1017.9, found [M+H]⁺=1019.2.

### Synthetic Example 9

### Bis(2-Hexyldecyl) 6,6′-((4,4′-(Methylazanediyl)Bis(Butanoyl))Bis(Isopropylazanediyl))Dihexanoate (Compound I-51)

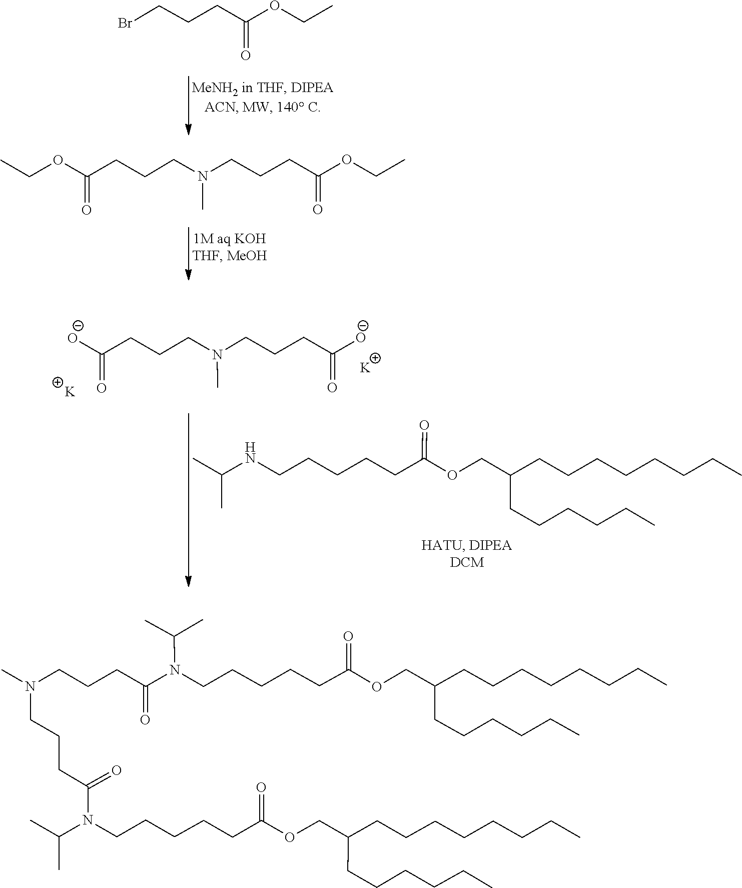

### Synthesis of diethyl 4,4′-(methylazanediyl)dibutyrate

Diethyl 4,4′-(methylazanediyl)dibutyrate was prepared from ethyl 4-bromobutyrate (15.4 mmol, 3.0 g, 2.22 mL), methyl amine (9.2 mmol, 2M THF solution, 4.61 mL) and DIPEA (30.8 mmol, 4.0 g, 5.37 mL) in ACN (8.0 mL), following the procedures outlined herein. Yield (650 mg, 33%). ESI-MS: m/z calculated for C₁₃H₂₅NO₄=259.2, found [M+H]⁺=260.3.

### Synthesis of dipotassium 4,4′-(methylazanediyl)dibutyrate

Diethyl 4,4′-(methylazanediyl)dibutyrate (2.39 mmol, 620 mg) was dissolved in 10 mL of 1:1 THF/MeOH mixture. To this solution, 7 ml of 1M aquas potassium hydroxide were added and the reaction mixture stirred for 2 days at room temperature. After that, the solvents were removed under vacuum and the residue taken in MeOH, filtered through small pad of Celite and concentrated under reduced pressure to give crude dipotassium 4,4′-(methylazanediyl)dibutyrate (650 mg, 97%). ESI-MS: m/z calculated for C₉H₁₅K₂NO₄=279.0, calculated for C₉H₁₇NO₄=203.1 (diacid), found [M+H]⁺=204.2.

### Synthesis of Compound I-51

Compound I-51 was prepared from dipotassium 4,4′-(methylazanediyl)dibutyrate (261 μmol, 72.9 mg), starting materials as shown (348 μmol, 138 mg), DIPEA (860 μmol, 112 mg, 152 μL) and HATU (522 μmol, 198 mg) by following the procedures outlined herein. Yield (55 mg, 33%).

¹H NMR (400 MHz, CDCl₃) δ 4.69-4.56 (m, 1H), 4.09-3.99 (m, 1H), 3.99-3.92 (m, 4H), 3.17-3.06 (m, 4H), 2.44-2.18 (m, 12H), 2.22 (s, 3H), 1.84-1.73 (m, 4H), 1.73-1.49 (m, 15H), 1.39-1.15 (m, 53H), 1.17 (d, J=6.6 Hz, 6H), 1.12 (d, J=6.9 Hz, 6H), 0.92-0.83 (m, 12H). ESI-MS: m/z calculated for C₅₉H₁₅N₃O₆=961.9, found [M+H]⁺=963.2.

### Synthetic Example 10

### Bis(2-Hexyldecyl) 10,10′-((4,4′-(Methylazanediyl)Bis(Butanoyl))Bis(Isopropylazanediyl))Bis(Decanoate) (Compound I-49)

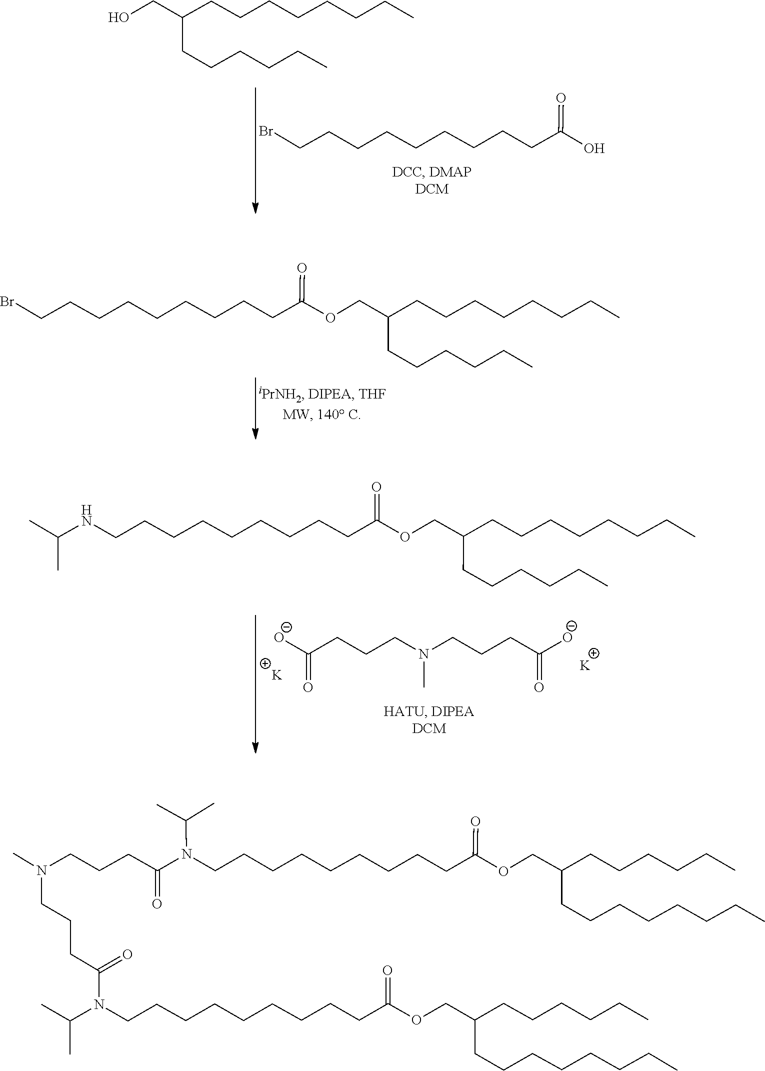

### Synthesis of 2-hexyldecyl 10-bromodecanoate

2-Hexyldecyl 10-bromodecanoate was prepared from 2-hexyl-1-decanol (8.76 mmol, 2.1 g), DMAP (796 μmol, 97.3 mg), 10-bromohexanoic acid (7.96 mmol, 2.0 g) and DCC (8.76 mmol, 1.8 g) by following the procedures outlined herein. Yield (3.4 g, 90%). ESI-MS: m/z calculated for C₂₆H₅₁BrO₂=474.3, found [M+H]⁺=475.4.

### Synthesis of 2-hexyldecyl 10-(isopropylamino)decanoate

2-Hexyldecyl 10-(isopropylamino)decanoate was prepared from 2-hexyldecyl 10-bromodecanoate (3.57 mmol, 1.7 g), isopropyl amine (5.36 mmol, 317 mg) and DIPEA (7.15 mmol, 924 mg, 1.25 mL), in ACN (10.7) by following the procedures outlined herein. Yield (1.0 g, 62%). ESI-MS: m/z calculated for C₂₉H₅₉NO₂=453.4, found [M+H]⁺=454.6.

### Synthesis of Compound I-49

Compound I-49 was prepared from starting material (786 μmol, 220 mg), 2-hexyldecyl 10-(isopropylamino)decanoate (605 μmol, 275 mg), DIPEA (2.42 mmol, 313 mg, 423 μL) and HATU (302 μmol, 115 mg) by following the procedures outlined herein. Yield (150 mg, 23%).

¹H NMR (400 MHz, CDCl₃) δ 4.64 (p, J=6.9 Hz, 1H), 4.04 (p, J=6.7 Hz, 1H), 3.96 (dd, J=5.8, 1.5 Hz, 4H), 3.19-3.02 (m, 4H), 2.48-2.16 (m, 15H), 1.92-1.44 (m, 19H), 1.40-1.20 (m, 68H), 1.17 (d, J=6.7 Hz, 6H), 1.12 (d, J=6.8 Hz, 6H), 0.88 (t, J=6.7 Hz, 12H). ESI-MS: m/z calculated for C₆₇H₁₃₁N₃O₆=1074.0, found [M+H]⁺=1075.2.

### Synthetic Example 11

### Bis(2-Hexyldecyl) 6,6′-((8,8′-(Methylazanediyl)Bis(Octanoyl))Bis(Hexylazanediyl))Dihexanoate (Compound I-25)

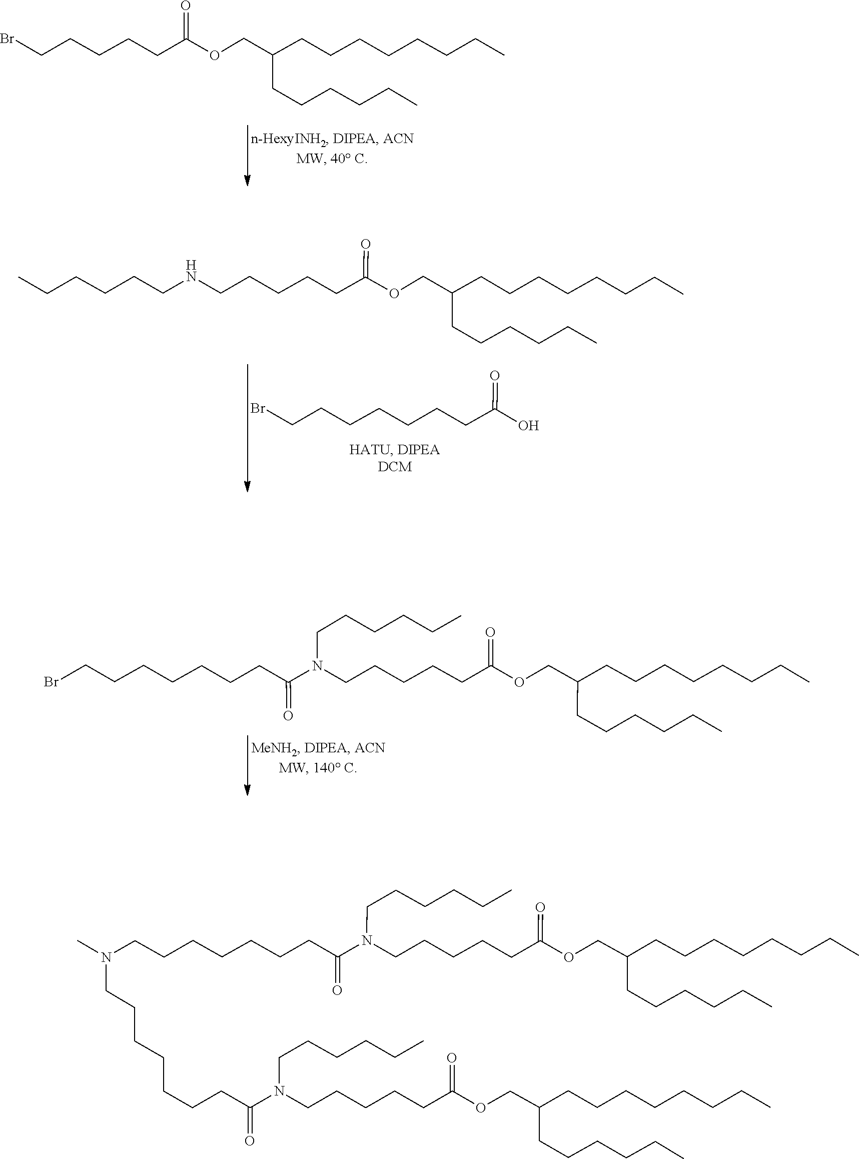

### Synthesis of 2-hexyldecyl 6-(hexylamino)hexanoate

2-Hexyldecyl 6-(hexylamino)hexanoate was prepared from 2-hexyldecyl 6-bromohexanoate (20.8 mmol, 8.72 g), hexyl amine (62.4 mmol, 6.31 g) and DIPEA (41.6 mmol, 5.37 g, 7.26 mL) in ACN (120.1 mL), by following the procedures outlined herein. Yield (5.6 g, 62%). ESI-MS: m/z calculated for C₂₈H₅₇NO₂=439.4, found [M+H]⁺=440.7.

### Synthesis of 2-hexyldecyl 6-(8-bromo-N-hexyloctanamido)hexanoate

2-Hexyldecyl 6-(8-bromo-N-hexyloctanamido)hexanoate from 2-hexyldecyl 6-(hexylamino)hexanoate (6.37 mmol, 2.8 g), 8-bromooctanoic acid (7.64 mmol, 1.49 g), DIPEA (12.7 mmol, 1.65 g, 2.22 mL) and HATU (8.28 mmol, 3.15 g) by following the procedures outlined herein. Yield (3.7 g, 94%). ESI-MS: m/z calculated for C₃₆H₇₀BrNO₃=643.4, found [M+H]⁺=644.6.

### Synthesis of Compound I-25

Compound I-25 was prepared from 2-hexyldecyl 6-(8-bromo-N-hexyloctanamido)hexanoate (330 μmol, 213 mg), methyl amine (198 μmol, 2M THF solution, 99 μL) and DIPEA (660 μmol, 85.3 mg, 115 μL) in ACN (0.30 mL), by following the procedures outlined herein. Yield (81.0 mg, 42%).

¹H NMR (400 MHz, CDCl₃) δ 3.99-3.92 (m, 4H), 3.31-3.24 (m, 4H), 3.23-3.15 (m, 4H), 2.38-2.21 (m, 12H), 2.18 (s, 3H), 1.71-1.58 (m, 15H), 1.58-1.38 (m, 13H), 1.37-1.19 (m, 80H), 0.95-0.81 (m, 18H). ESI-MS: m/z calculated for C₇₃H₁₄₃N₃O₆=1158.1, found [M+H]⁺=1159.3.

### Synthetic Example 12

### Rel-Bis(2-Hexyldecyl) 6,6′-((8,8′-(((1R,4R)-4-Hydroxycyclohexyl)Azanediyl)Bis(Octanoyl))Bis(Hexylazanediyl))Dihexanoate (Compound I-12)

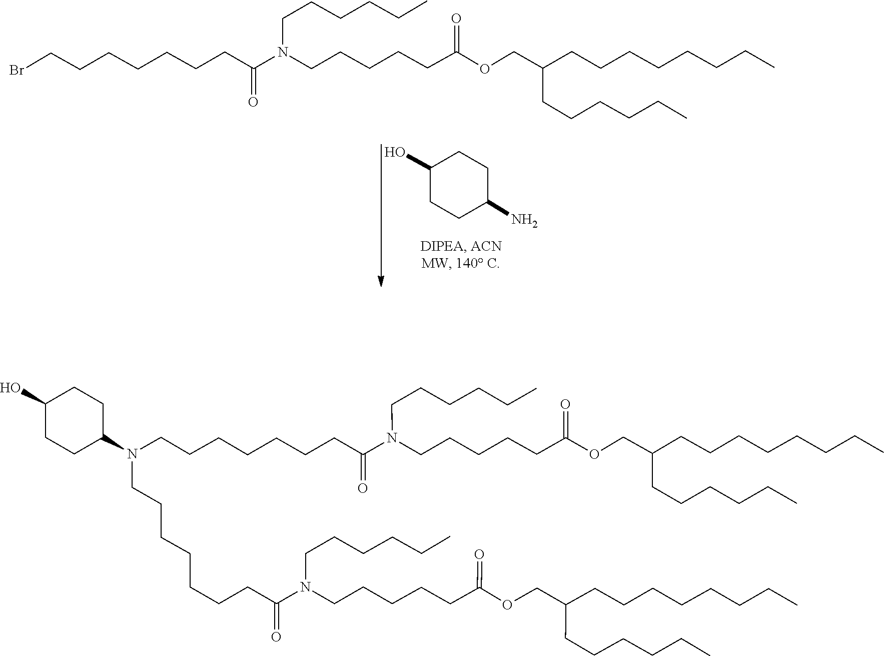

### Synthesis of Compound I-12

Compound I-12 was prepared from 2-hexyldecyl 6-(8-bromo-N-hexyloctanamido)hexanoate (419 μmol, 270 mg), cis-4-aminocyclohexanol (230 μmol, 26.5 mg) and DIPEA (837 μmol, 108.0 mg, 146 μL) in ACN (0.82), by following the procedures outlined herein. Yield (70.0 mg, 27%).

¹H NMR (400 MHz, CDCl₃) δ 4.01-3.89 (m, 5H), 3.36-3.25 (m, 4H), 3.25-3.14 (m, 4H), 2.46-2.38 (m, 5H), 2.36-2.22 (m, 8H), 1.83 (d, J=13.0 Hz, 2H), 1.74-1.60 (m, 17H), 1.58-1.45 (m, 13H), 1.44-1.21 (m, 79H), 0.94-0.83 (m, 18H). ESI-MS: m/z calculated for C₇₈H₁₅₁N₃O₇=1242.2, found [M+H]⁺=1243.5.

### Synthetic Example 13

### Bis(2-Hexyldecyl) 6,6′-((6,6′-(Methylazanediyl)Bis(Hexanoyl))Bis(Hexylazanediyl))Dihexanoate (Compound I-27)

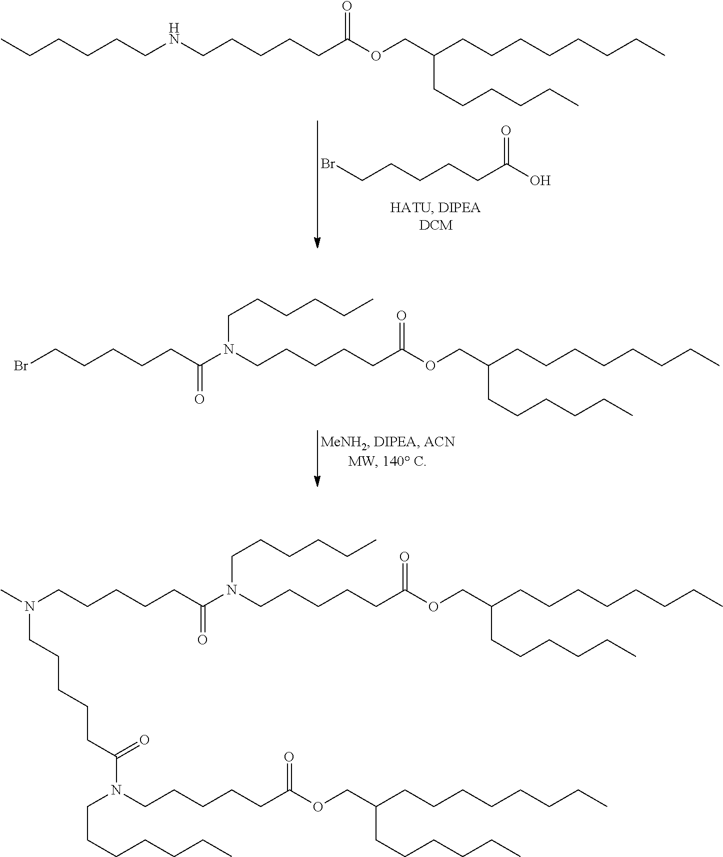

### Synthesis of 2-hexyldecyl 6-(6-bromo-N-hexylhexanamido)hexanoate

2-Hexyldecyl 6-(6-bromo-N-hexylhexanamido)hexanoate was prepared from 2-hexyldecyl 6-(hexylamino)hexanoate (373 μmol, 373 mg), 8-bromohexanoic acid (448 μmol, 87.3 mg), DIPEA (746 μmol, 96.4 mg, 130 μL) and HATU (485 μmol, 184 mg) by following the procedures outlined herein. Yield (210 mg, 91%). ESI-MS: m/z calculated for C₃₄H₆₆BrNO₃=615.4, found [M+H]⁺=616.6.

### Synthesis of Compound I-27

Compound I-27 was prepared from 2-hexyldecyl 6-(6-bromo-N-hexylhexanamido)hexanoate (308 μmol, 190 mg), methyl amine (185 μmol, 2M THF solution, 92 μL) and DIPEA (616 μmol, 79.6 mg, 108 μL) in ACN (0.30 mL), by following the procedures outlined herein. Yield (85.0 mg, 50%).

¹H NMR (400 MHz, CDCl₃) δ 3.99-3.92 (m, 4H), 3.31-3.24 (m, 4H), 3.23-3.11 (m, 4H), 2.38-2.21 (m, 12H), 2.18 (s, 3H), 1.71-1.58 (m, 11H), 1.58-1.38 (m, 12H), 1.37-1.19 (m, 70H), 0.93-0.83 (m, 18H). ESI-MS: m/z calculated for C₆₉H₁₃₅N₃O₆=1102.0, found [M+H]⁺=1103.3.

### Synthetic Example 14

### Bis(2-Hexyldecyl) 6,6′-((6,6′-((3-Methoxypropyl)Azanediyl)Bis(Hexanoyl))Bis(Hexylazanediyl))Dihexanoate (Compound I-37)

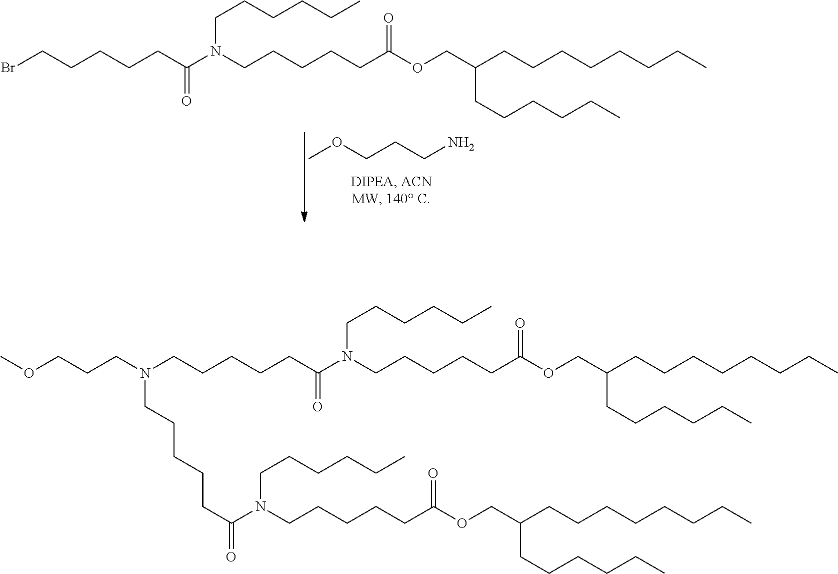

### Synthesis of Compound I-37

The title compound was prepared from 2-hexyldecyl 6-(6-bromo-N-hexylhexanamido)hexanoate (674 μmol, 415 mg), 3-methoxy-1-propylamine (370 μmol, 33.0 mg) and DIPEA (1.35 mmol, 174.0 mg, 235 μL) in ACN (1.05 mL), by following the procedures outlined herein. Yield (150.0 mg, 38%).

¹H NMR (400 MHz, CDCl₃) δ 3.96 (dd, J=7.6, 5.8 Hz, 4H), 3.38 (t, J=6.4 Hz, 2H), 3.31 (s, 3H), 3.31-3.24 (m, 4H), 3.22-3.11 (m, 4H), 2.50-2.36 (m, 6H), 2.35-2.20 (m, 8H), 1.74-1.38 (m, 26H), 1.36-1.19 (m, 68H), 0.94-0.83 (m, 18H). ESI-MS: m/z calculated for C₇₂H₁₄₁N₃O₇=1160.1, found [M+H]⁺=1161.2.

### Synthetic Example 15

### Bis(2-Hexyldecyl) 6,6′-((4,4′-(Methylazanediyl)Bis(Butanoyl))Bis(Hexylazanediyl))Dihexanoate (Compound I-52)

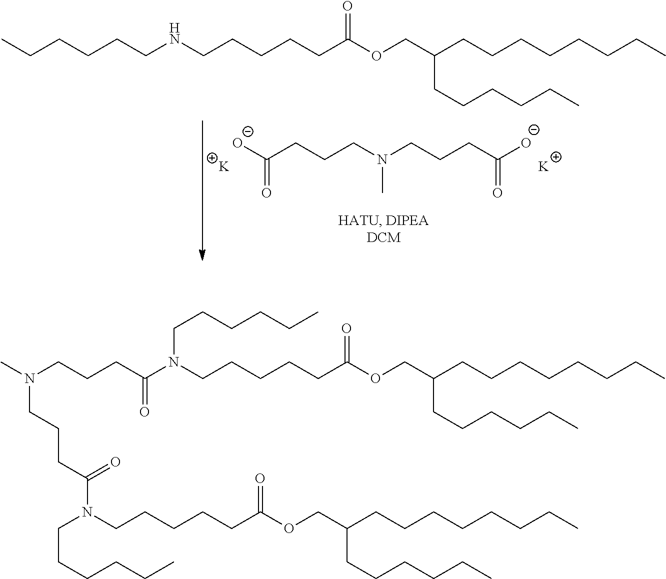

### Synthesis of Compound I-52

The title compound was prepared from 2-hexyldecyl 6-(hexylamino)hexanoate (355 μmol, 156 mg), dipotassium 4,4′-(methylazanediyl)dibutyrate (266 μmol, 74 mg), DIPEA (887 μmol, 115 mg, 155 μL) and HATU (532 μmol, 202 mg) by following the procedures outlined herein. Yield (80 mg, 43%).

¹H NMR (400 MHz, CDCl₃) δ 3.97 (t, J=6.3 Hz, 4H), 3.39-3.08 (m, 8H), 2.42-2.27 (m, 12H), 2.23 (s, 3H), 1.80 (p, J=7.4 Hz, 4H), 1.72-1.45 (m, 19H), 1.36-1.17 (m, 62H), 0.95-0.73 (m, 18H). ESI-MS: m/z calculated for C₆₅H₁₂₇N₃O₆=1046.0, found [M+H]⁺=1047.2.

### Synthetic Example 16

### Bis(2-Hexyldecyl) 6,6′-((7,7′-(Methylazanediyl)Bis(Heptanoyl))Bis(Hexylazanediyl))Dihexanoate (Compound I-24)

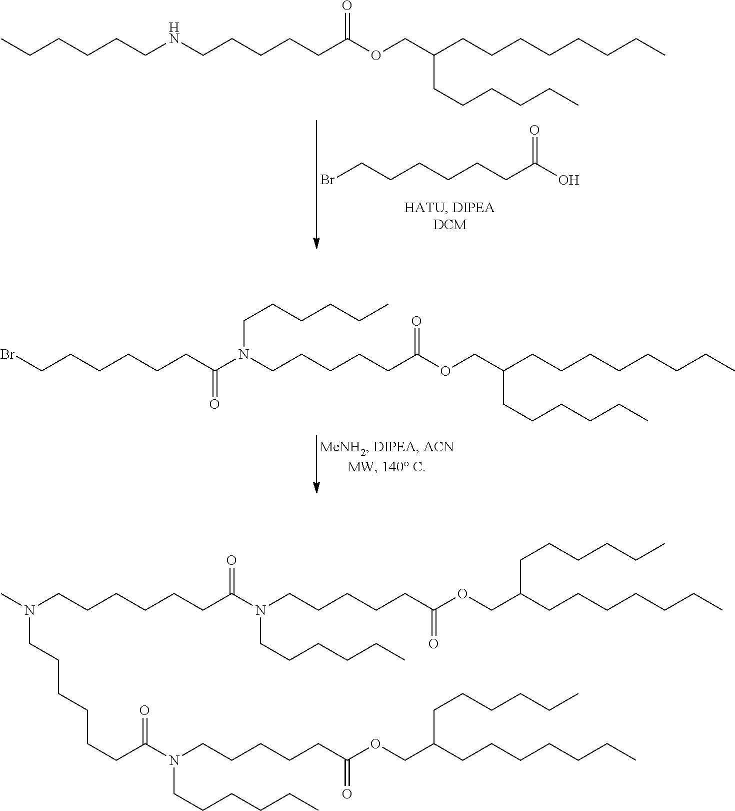

### Synthesis of 2-hexyldecyl 6-(7-bromo-N-hexylheptanamido)hexanoate

2-Hexyldecyl 6-(7-bromo-N-hexylheptanamido)hexanoate was prepared from 2-hexyldecyl 6-(hexylamino)hexanoate (300 μmol, 132 mg), 8-bromoheptanoic acid (330 μmol, 69 mg), DIPEA (600 μmol, 77 mg, 105 μL) and HATU (390 μmol, 148 mg) by following the procedures outlined herein. Yield (150 mg, 79%). ESI-MS: m/z calculated for C₃₅H₆₈BrNO₃=629.4, found [M+H]⁺=630.6.

### Synthesis of Compound I-24

The title compound was prepared from 2-hexyldecyl 6-(7-bromo-N-hexylheptanamido)hexanoate (234 μmol, 148 mg), methyl amine (140 μmol, 2M THE solution, 70 μL) and DIPEA (468 μmol, 81 mg, 60 μL) in ACN (0.2 mL), by following the procedures outlined herein. Yield (35.1 mg, 27%).

¹H NMR (400 MHz, CDCl₃) δ 3.97 (dd, J=7.8, 5.8 Hz, 4H), 3.28 (t, J=7.7 Hz, 4H), 3.23-3.14 (m, 4H), 2.36-2.22 (m, 12H), 2.18 (s, 3H), 1.72-1.60 (m, 16H), 1.50 (ddt, J=27.5, 14.5, 7.4 Hz, 12H), 1.38-1.21 (m, 74H), 0.94-0.83 (m, 18H). ESI-MS: m/z calculated for C₇₁H₁₃₉N₃O₆=1130.1, found [M+H]⁺=1131.3.

### Synthetic Example 17

### Bis(2-Hexyldecyl) 6,6′-((3,3′-(Methylazanediyl)Bis(Propanoyl))Bis(Hexylazanediyl))Dihexanoate (Compound I-48)

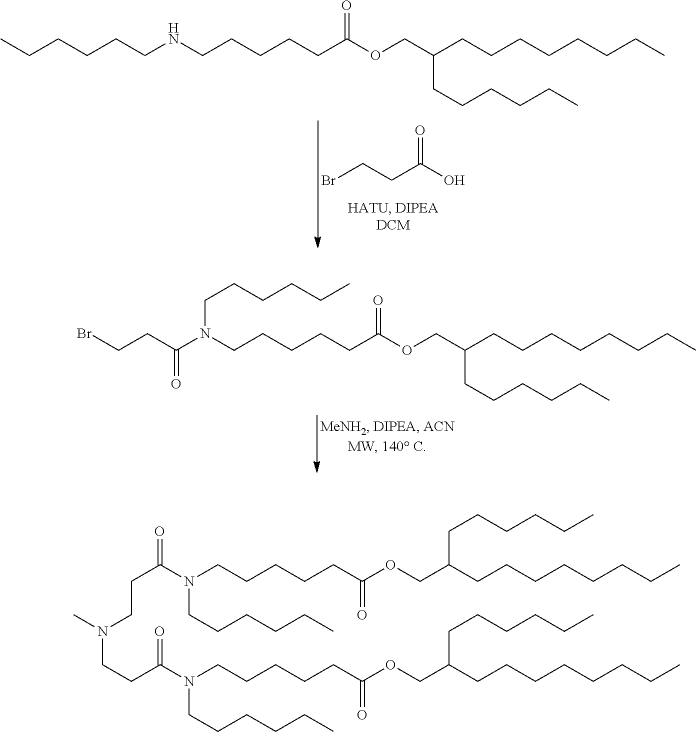

### Synthesis of 2-hexyldecyl 6-(3-bromo-N-hexylpropanamido)hexanoate

2-Hexyldecyl 6-(3-bromo-N-hexylpropanamido)hexanoate was prepared from 2-hexyldecyl 6-(hexylamino)hexanoate (682 μmol, 300 mg), 8-bromopropanoic acid (750 μmol, 115 mg), DIPEA (1.36 mmol, 176 mg, 238 μL) and HATU (887 μmol, 337 mg) by following the procedures outlined herein. Yield (160 mg, 41%). ESI-MS: m/z calculated for C₃₁H₆₀BrNO₃=573.4, found [M+H]⁺=574.5.

### Synthesis of Compound I-48

The title compound was prepared from 2-hexyldecyl 6-(3-bromo-N-hexylpropanamido)hexanoate (278 μmol, 160 mg), methyl amine (167 μmol, 2M THF solution, 84 μL) and DIPEA (557 μmol, 72 mg, 97 μL) in ACN (0.25 mL), by following the procedures outlined herein. Yield (40 mg, 28%).

¹H NMR (400 MHz, CDCl₃) δ 3.96 (t, J=5.9 Hz, 4H), 3.32-3.16 (m, 8H), 2.81-2.71 (m, 4H), 2.53-2.41 (m, 4H), 2.35-2.24 (m, 7H), 1.72-1.45 (m, 20H), 1.38-1.23 (m, 66H), 0.94-0.83 (m, 18H). ESI-MS: m/z calculated for C₆₃H₁₂₃N₃O₆=1017.9, found [M+H]⁺=1019.2.

### Synthetic Example 18

### Bis(2-Hexyldecyl) 6,6′-((10,10′-(Methylazanediyl)Bis(Decanoyl))Bis(Hexylazanediyl))Dihexanoate (Compound I-21)

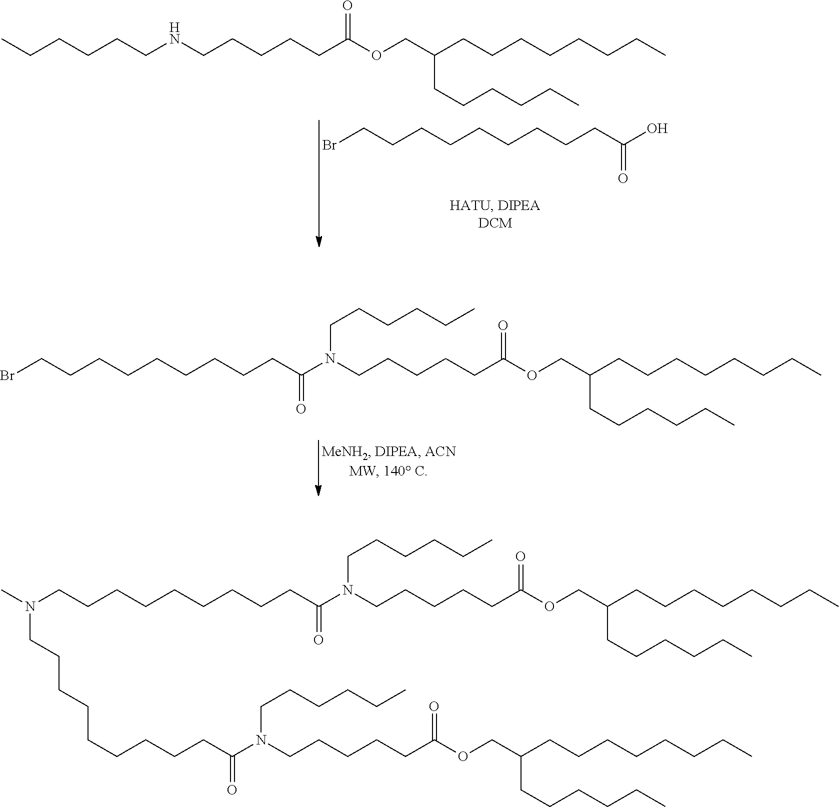

### Synthesis of 2-hexyldecyl 6-(10-bromo-N-hexyldecanamido)hexanoate

2-hexyldecyl 6-(10-bromo-N-hexyldecanamido)hexanoate was prepared from 2-hexyldecyl 6-(hexylamino)hexanoate (318 μmol, 140 mg), 8-bromodecanoic acid (350 μmol, 88 mg), DIPEA (637 μmol, 82 mg, 111 μL) and HATU (414 μmol, 157 mg) by following the procedures outlined herein. Yield (193 mg, 90%). ESI-MS: m/z calculated for C₃₈H₇₄BrNO₃=671.5, found [M+H]⁺=672.6.

### Synthesis of Compound I-21

The title compound was prepared from 2-hexyldecyl 6-(10-bromo-N-hexyldecanamido)hexanoate (208 μmol, 140 mg), methyl amine (125 μmol, 2M THF solution, 63 μL) and DIPEA (416 μmol, 54 mg, 73 μL) in ACN (0.19 mL), by following the procedures outlined herein. Yield (55 mg, 44%).

¹H NMR (400 MHz, CDCl₃) δ 3.96 (t, J=5.8 Hz, 4H), 3.32-3.16 (m, 8H), 2.82-2.72 (m, 4H), 2.48 (td, J=7.7, 4.0 Hz, 4H), 2.35-2.25 (m, 7H), 1.71-1.44 (m, 28H), 1.38-1.20 (m, 71H), 0.94-0.83 (m, 18H). ESI-MS: m/z calculated for C₇₇H₁₅₁N₃O₆=1214.2, found [M+H]⁺=1215.4.

### Synthetic Example 19

### Bis(2-Hexyldecyl) 6,6′-((9,9′-(Methylazanediyl)Bis(Nonanoyl))Bis(Hexylazanediyl))Dihexanoate (Compound I-20)

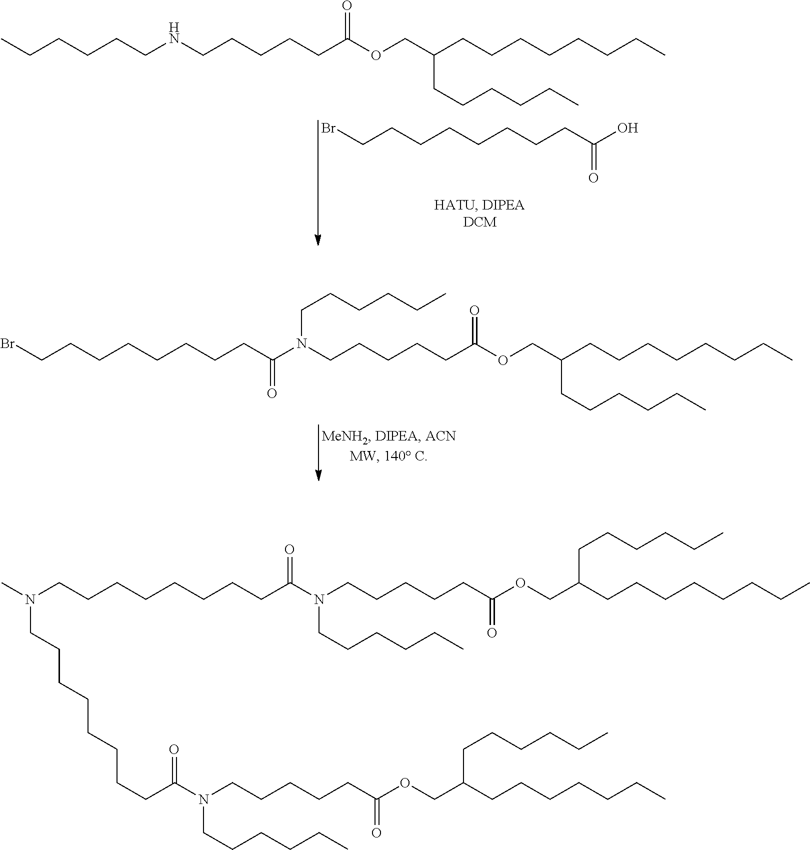

### Synthesis of 2-hexyldecyl 6-(10-bromo-N-hexylnonanamido)hexanoate

2-hexyldecyl 6-(10-bromo-N-hexylnonanamido)hexanoate was prepared from 2-hexyldecyl 6-(hexylamino)hexanoate (327 μmol, 144 mg), 8-bromononanoic acid (360 μmol, 85 mg), DIPEA (655 μmol, 85 mg, 114 μL) and HATU (426 μmol, 162 mg) by following the procedures outlined herein. Yield (210 mg, 97%). ESI-MS: m/z calculated for C₃₇H₇₂BrNO₃=657.5, found [M+H]⁺=658.6.

### Synthesis of Compound I-20

The title compound was prepared from 2-hexyldecyl 6-(10-bromo-N-hexylnonanamido)hexanoate (304 μmol, 200 mg), methyl amine (182 μmol, 2M THF solution, 91 μL) and DIPEA (607 μmol, 79 mg, 106 μL) in ACN (0.27 mL), by following the procedures outlined herein. Yield (60 mg, 33%).

¹H NMR (400 MHz, CDCl₃) δ 3.96 (dd, J=8.0, 5.8 Hz, 4H), 3.32-3.14 (m, 8H), 2.43-2.21 (m, 12H), 2.19 (s, 3H), 1.55 (dddd, J=55.4, 25.6, 15.5, 7.7 Hz, 29H), 1.27 (dt, J=12.4, 6.7 Hz, 84H), 0.94-0.83 (m, 18H). ESI-MS: m/z calculated for C₇₅H₁₄₇N₃O₆=1186.1, found [M+H]⁺=1187.4.

### Synthetic Example 20

### Bis(2-Hexyldecyl) 6,6′-((5,5′-(Methylazanediyl)Bis(Pentanoyl))Bis(Hexylazanediyl))Dihexanoate (Compound I-19)

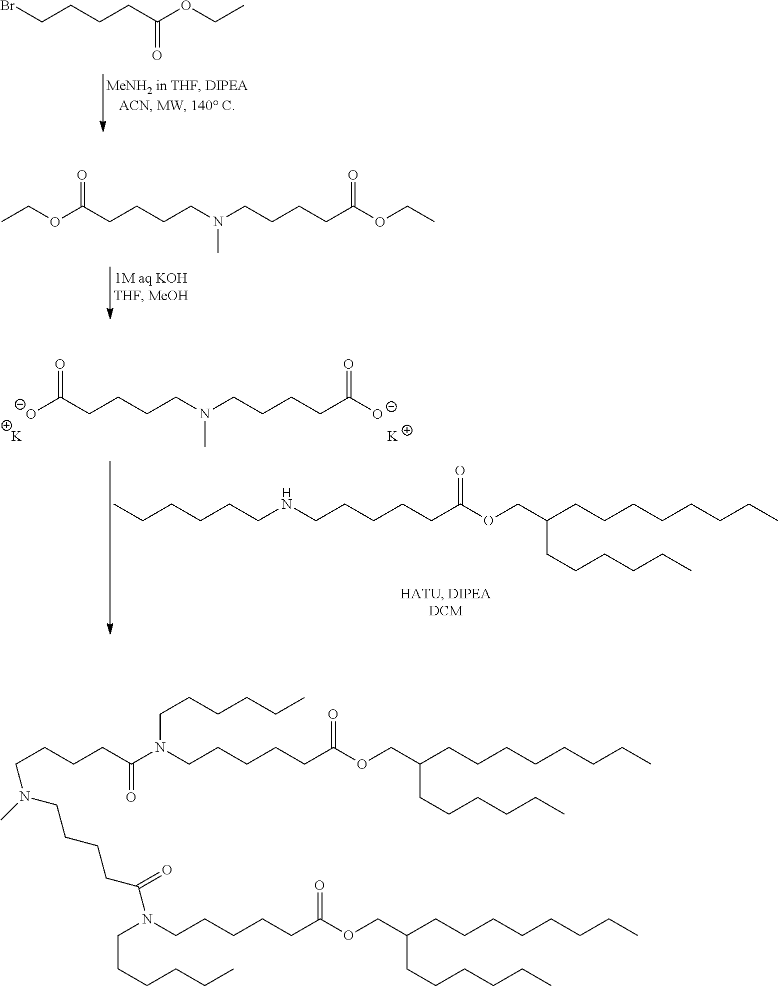

### Synthesis of diethyl 5,5′-(methylazanediyl)dipentanoate

Diethyl 5,5′-(methylazanediyl)dipentanoate was prepared from ethyl 2-bromovalerate (19.1 mmol, 4.0 g), methyl amine (11.5 mmol, 2M THF solution, 5.74 mL) and DIPEA (38.3 mmol, 4.9 g, 6.7 mL) in ACN (6 mL), following the procedures outlined herein. Yield (850 mg, 31%). ESI-MS: m/z calculated for C₁₅H₂₉NO₄=287.2, found [M+H]⁺=288.3.

### Synthesis of dipotassium 5,5′-(methylazanediyl)dipentanoate

Dipotassium 5,5′-(methylazanediyl)dipentanoate was prepared from diethyl 5,5′-(methylazanediyl)dipentanoate (2.96 mmol, 850 mg), by following the procedures outlined herein. Yield (909 mg, 89%). ESI-MS: m/z calculated for C₁₁H₁₉K₂NO₄=307.1, calculated for C₁₁H₂₁NO₄=231.1 (diacid), found [M+H]⁺=232.3.

### Synthesis of Compound I-19

The title compound was prepared from dipotassium 5,5′-(methylazanediyl)dipentanoate (294 mol, 91 mg), 2-hexyldecyl 6-(hexylamino)hexanoate (882 μmol, 388 mg), DIPEA (1.47 mmol, 190 mg, 257 μL) and HATU (764 μmol, 291 mg) by following the procedures outlined herein. Yield (35 mg, 11%)

¹H NMR (400 MHz, CDCl₃) δ 3.96 (dd, J=7.2, 5.8 Hz, 4H), 3.32-3.14 (m, 8H), 3.37-3.26 (m, 12H), 2.19 (s, 3H), 1.72-1.43 (m, 27H), 1.39-1.22 (m, 68H), 0.94-0.83 (m, 18H). ESI-MS: m/z calculated for C₆₇H₁₃₁N₃O₆=1074.0, found [M+H]⁺=1075.2.

### Synthetic Example 21

### Bis(2-Hexyldecyl) 10,10′-((4,4′-(Methylazanediyl)Bis(Butanoyl))Bis(Hexylazanediyl))Bis(Decanoate) (Compound I-50)

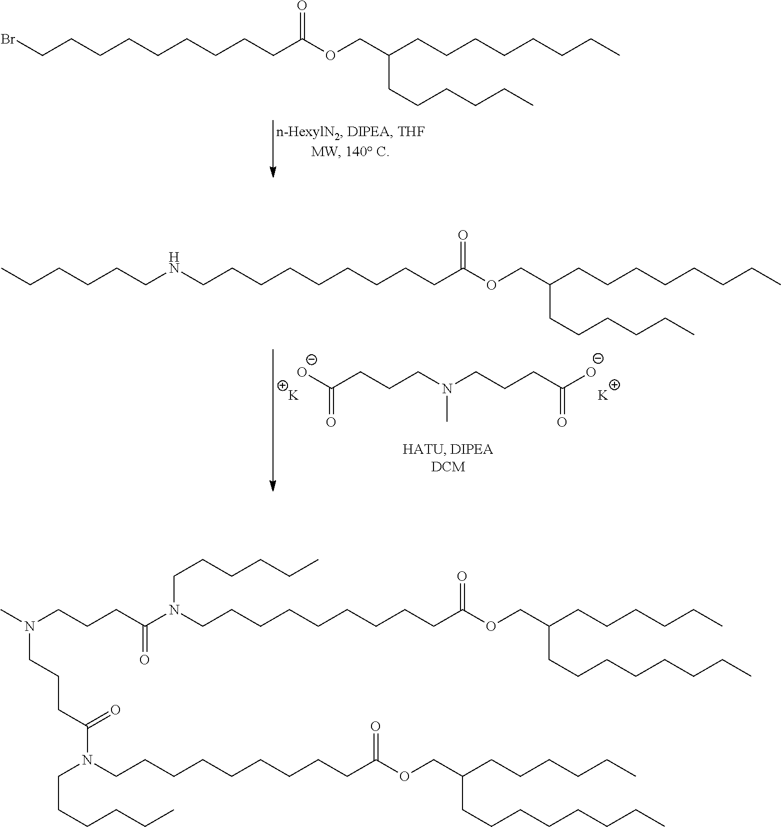

### Synthesis of 2-hexyldecyl 10-(hexylamino)decanoate

2-Hexyldecyl 10-(hexylamino)decanoate was prepared from 2-hexyldecyl 10-bromodecanoate (3.26 mmol, 1.6 g), hexyl amine (4.89 mmol, 495 mg) and DIPEA (6.52 mmol, 842 mg, 1.14 mL) in ACN (15 mL), by following the procedures outlined herein. Yield (1.0 g, 63%). ESI-MS: m/z calculated for C₃₂H₆₅NO₂=495.5, found [M+H]⁺=496.4.

### Synthesis of Compound I-50

The title compound was prepared from dipotassium 4,4′-(methylazanediyl)dibutyrate (393 mol, 110 mg), 2-hexyldecyl 10-(hexylamino)decanoate (605 μmol, 300 mg), DIPEA (1.21 mmol, 156 mg, 212 μL) and HATU (1.51 mmol, 575 mg) by following the procedures outlined herein. Yield (60 mg, 17%).

¹H NMR (400 MHz, CDCl₃) δ 3.96 (dd, J=5.7, 1.4 Hz, 4H), 3.32-3.15 (m, 8H), 2.41-2.20 (m, 14H), 1.80 (p, J=7.5 Hz, 4H), 1.69-1.56 (m, 13H), 1.56-1.42 (m, 9H), 1.28 (dd, J=10.6, 4.8 Hz, 80H), 0.89 (td, J=8.0, 5.5 Hz, 18H). ESI-MS: m/z calculated for C₇₃H₁₄₃N₃O₆=1158.1, found [M+H]⁺=1159.3.

### Synthetic Example 22

### Bis(2-Hexyldecyl) 10,10′-((6,6′-(Methylazanediyl)Bis(Hexanoyl))Bis(Hexylazanediyl))Bis(Decanoate) (Compound I-22)

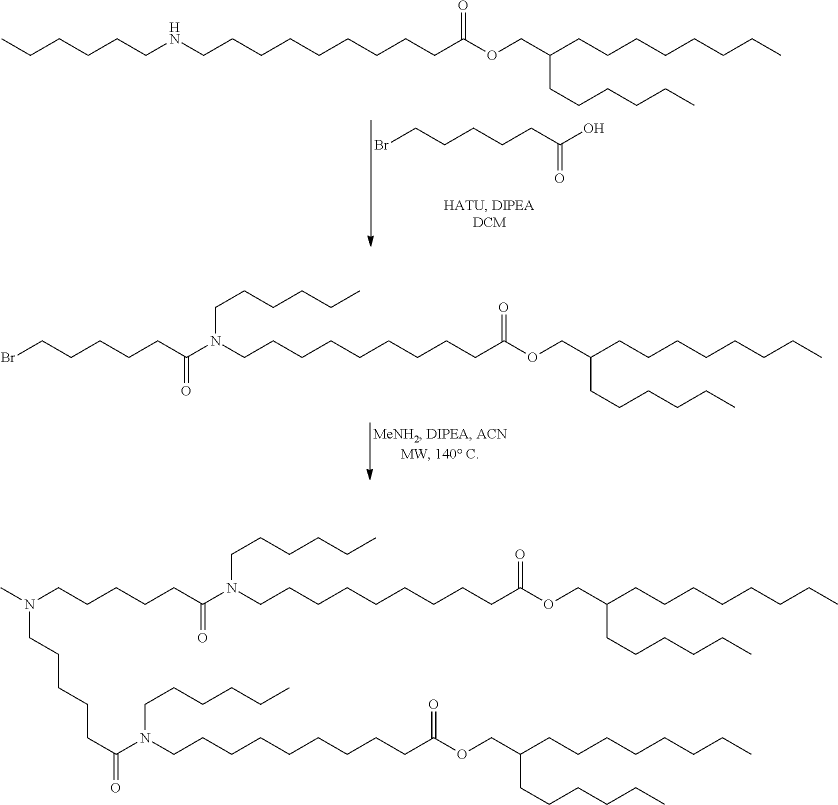

### Synthesis of 2-hexyldecyl 6-(6-bromo-N-hexylhexanamido)hexanoate

2-Hexyldecyl 6-(6-bromo-N-hexylhexanamido)hexanoate was prepared from 2-hexyldecyl 10-(hexylamino)decanoate (334 μmol, 166 mg), 8-bromohexanoic acid (368 μmol, 72 mg), DIPEA (668 μmol, 86 mg, 117 μL) and HATU (434 μmol, 165 mg) by following the procedures outlined herein. Yield (200 mg, 89%). ESI-MS: m/z calculated for C₃₈H₇₄BrNO₃=671.5, found [M+H]⁺=672.7.

### Synthesis of Compound I-22

The title compound was prepared from 2-hexyldecyl 6-(6-bromo-N-hexylhexanamido)hexanoate (297 μmol, 200 mg), methyl amine (163 μmol, 2M THE solution, 82 μL) and DIPEA (149 μmol, 77 mg, 104 μL) in ACN (0.27 mL), by following the procedures outlined herein. Yield (60 mg, 33%).

¹H NMR (400 MHz, CDCl₃) δ 3.96 (dd, J=5.8, 1.8 Hz, 4H), 3.32-3.14 (m, 8H), 2.35-2.22 (m, 12H), 2.19 (s, 3H), 1.71-1.56 (m, 16H), 1.55-1.40 (m, 12H), 1.38-1.20 (m, 85H), 0.94-0.83 (m, 18H). ESI-MS: m/z calculated for C₇₇H₁₅₁N₃O₆=1214.2, found [M+H]⁺=1215.4.

### Synthetic Example 23

### ((8,8′-(Methylazanediyl)Bis(Octanoyl))Bis(Ethylazanediyl))Bis(Pentane-5,1-Diyl) Bis(2-Hexyldecanoate) (Compound I-29)

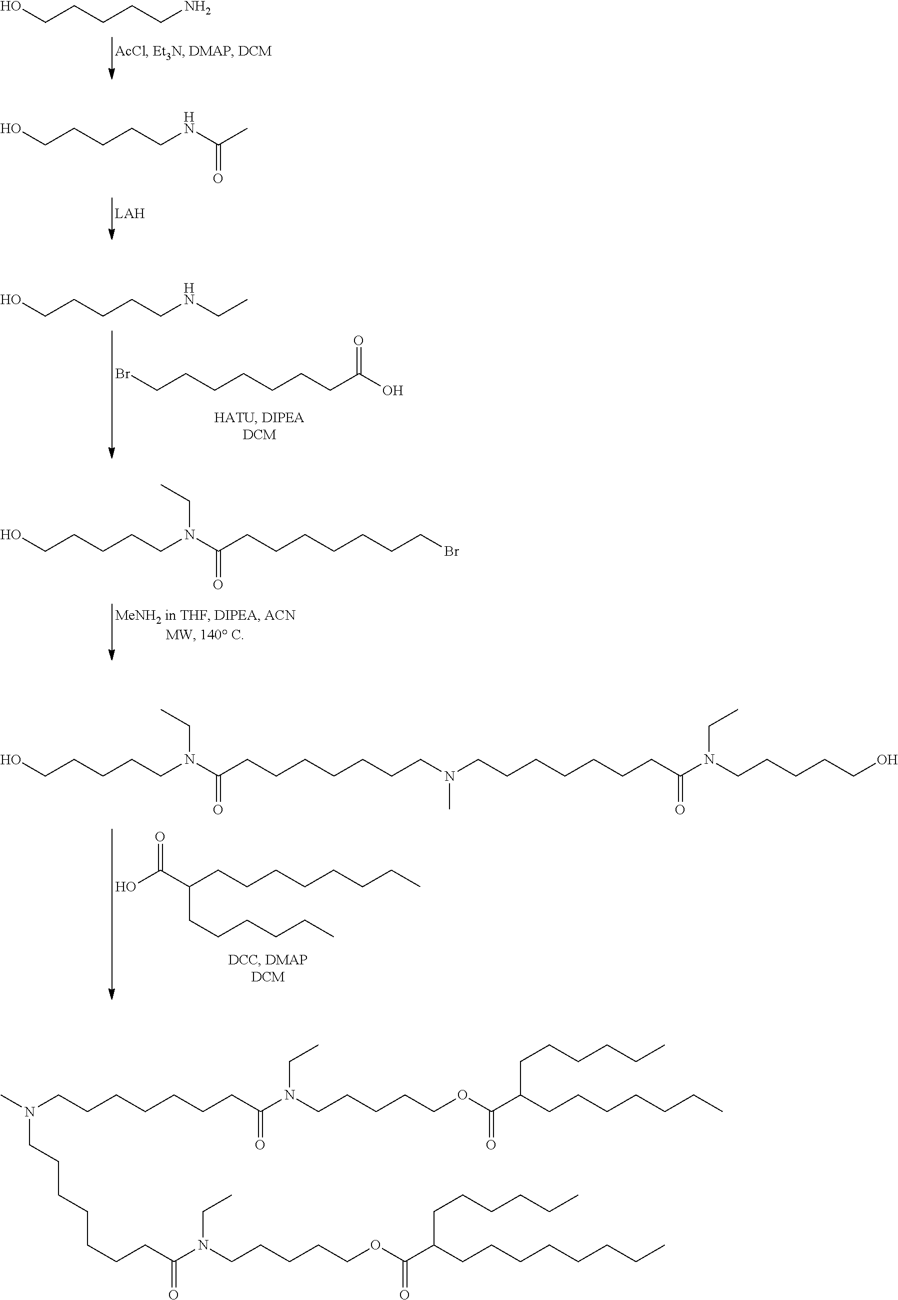

### Synthesis of N-(5-hydroxypentyl)acetamide

To a solution of 5-aminopentan-1-ol (4.8 mmol, 500 mg), DMAP (242 μmol, 29.6 g) and triethyl amine (24.2 mmol, 2.45 g, 3.38 mL) in DCM (10 mL), cooled to 0° C. (ice bath), was dropwise added commercial acetyl chloride (19.4 mmol, 1.52 g, 1.38 mL) dissolved in 5 mL of DCM. The reaction mixture was stirred at room temperature for 30 min and then quenched by addition of 1 mL of MeOH. Solvent was removed under vacuum. Residue was taken in EtOAc, washed with water and dried over anhydrous Na₂SO₄. The crude N-(5-hydroxypentyl)acetamide (850 mg, 94%), obtained after the removal of solvent, was used in the next step as is. to give. ESI-MS: m/z calculated for C₇H₁₅NO₂=145.1, found [M+H]⁺=146.2.

### Synthesis of 5-(ethylamino)pentan-1-ol

To a solution of N-(5-hydroxypentyl)acetamide (4.85 mmol, 850 mg) in dry THF, LAH (24.2 mmol, 920 mg) was added in 1 portion. The reaction mixture was refluxed over night, cooled down and quenched with wet diethyl ether (10 ml). Quenched mixture was diluted with 30 mL of diethyl ether, filtered through small pad of Celite and dried over anhydrous Na₂SO₄. Removal of solvent under vacuum gave crude 5-(ethylamino)pentan-1-ol (400 mg, 63%), which was used in the next step as is. ESI-MS: m/z calculated for C₇H₁₇NO=131.1, found [M+H]⁺=132.1.

### Synthesis of 8-bromo-N-ethyl-N-(5-hydroxypentyl)octanamide

8-Bromo-N-ethyl-N-(5-hydroxypentyl)octanamide was prepared from 5-(ethylamino)pentan-1-ol (3.06 mmol, 400 mg), 8-bromooctanoic acid (2.78 mmol, 620 mg), DIPEA (5.56 mmol, 718 mg, 971 μL) and HATU (3.61 mmol, 1.37 g) by following the procedures outlined herein. Yield (450 mg, 48%). ESI-MS: m/z calculated for C₁₅H₃₀BrNO₂=335.1, found [M+H]⁺=336.3.

### Synthesis of 8,8′-(methylazanediyl)bis(N-ethyl-N-(5-hydroxypentyl)octanamide)

8,8′-(Methylazanediyl)bis(N-ethyl-N-(5-hydroxypentyl)octanamide) was prepared from 8-bromo-N-ethyl-N-(5-hydroxypentyl)octanamide (898 μmol, 302 mg), methyl amine (539 μmol, 2M THF solution, 269 μL) and DIPEA (1.80 mmol, 232 mg, 314 μL) in ACN (1.1 mL), by following the procedures outlined herein. Yield (115 mg, 47%). ESI-MS: m/z calculated for C₃₁H₆₃N₃O₄=541.5, found [M+H]⁺=542.6.

### Synthesis of Compound I-29

The title compound was prepared from 8,8′-(methylazanediyl)bis(N-ethyl-N-(5-hydroxypentyl)octanamide) (212 μmol, 115 mg), 2-hexyldecanoic acid (244 μmol, 62 mg, 72 μL), DCC (318 mmol, 66 mg) and DMAP (21 μmol, 3 mg) by following the procedures outlined herein. Yield (160 mg, 74%)

¹H NMR (400 MHz, CDCl₃) δ ESI-MS: 4.06 (dt, J=10.5, 6.5 Hz, 4H), 3.41-3.17 (m, 8H), 2.36-2.22 (m, 10H), 2.18 (s, 3H), 1.71-1.39 (m, 29H), 1.39-1.19 (m, 58H), 1.16 (t, J=7.1 Hz, 3H), 1.09 (t, J=7.1 Hz, 3H), 0.87 (t, J=6.7 Hz, 12H). ESI-MS: m/z calculated for C₆₃H₁₂₃N₃O₆=1017.9, found [M+H]⁺=1019.2.

### Synthetic Example 24

### Bis(2-Hexyldecyl) 8,8′-((5,5′-(Methylazanediyl)Bis(Pentanoyl))Bis(Hexylazanediyl))Dioctanoate (Compound I-18)

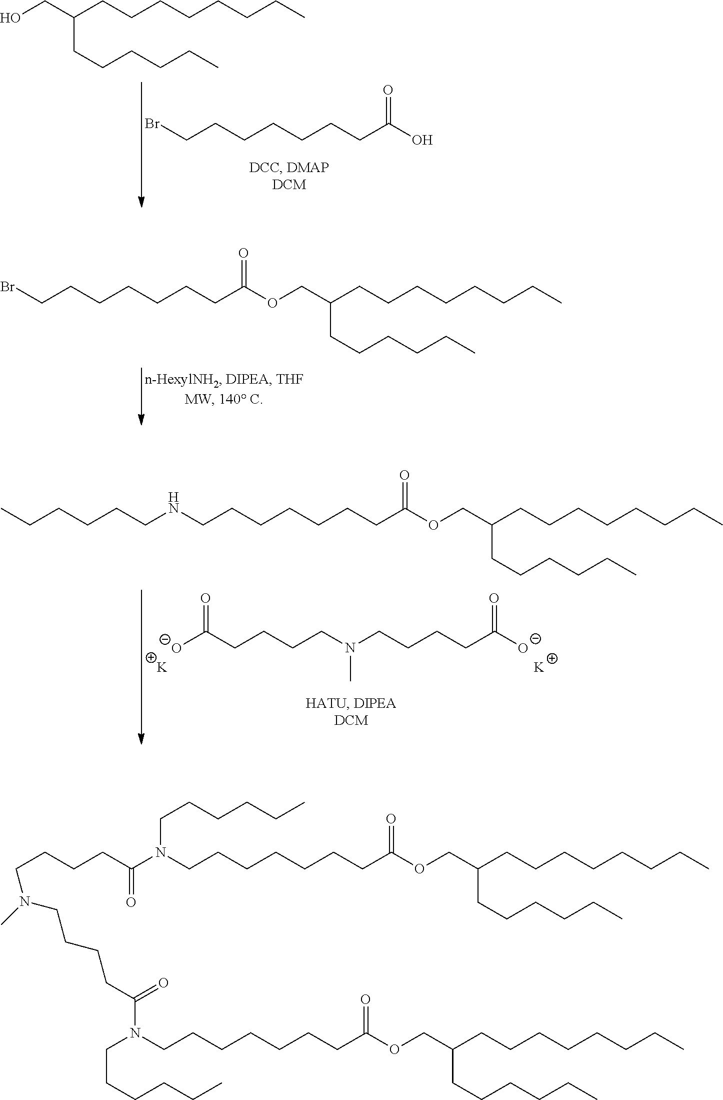

### Synthesis of 2-hexyldecyl 8-bromooctanoate

2-Hexyldecyl 8-bromooctanoate was prepared from 8-bromooctanoic acid (8.96 mmol, 2.00 g), 2-hexyldecan-1-ol (9.86 mmol, 2.39 g, 2.87 mL), DCC (9.86 mmol, 2.03 g) and DMAP (896 μmol, 110 mg) by following the procedures outlined herein. Yield (3.50 g, 87%). ESI-MS: m/z calculated for C₂₄H₄₇BrO₂=446.3, found [M+H]⁺=447.4.

### Synthesis of 2-hexyldecyl 8-(hexylamino)octanoate

2-Hexyldecyl 8-(hexylamino)octanoate was prepared from 2-hexyldecyl 8-bromooctanoate (4.47 mmol, 2.00 g), hexyl amine (8.94 mmol, 904 μL) and DIPEA (8.9 mmol, 1.16 g, 1.6 mL) in ACN (1.1 mL), by following the procedures outlined herein. Yield (1.10 g, 53%). ESI-MS: m/z calculated for C₃₀H₆₁NO₂=467.5, found [M+H]⁺=468.6.

### Synthesis of Compound I-18

The title compound was prepared from dipotassium 5,5′-(methylazanediyl)dipentanoate (182 mol, 56 mg), 2-hexyldecyl 8-(hexylamino)octanoate (545 μmol, 255 mg), DIPEA (908 μmol, 117 mg, 159 μL) and HATU (472 μmol, 180 mg) by following the procedures outlined herein. Yield (130 mg, 63%)

¹H NMR (400 MHz, CDCl₃) δ 3.96 (dd, J=5.8, 3.4 Hz, 4H), 3.32-3.14 (m, 8H), 2.37-2.24 (m, 12H), 2.19 (s, 3H), 1.69-1.43 (m, 29H), 1.36-1.21 (dd, J=12.4, 3.8 Hz, 74H), 0.94-0.83 (m, 18H). ESI-MS: m/z calculated for C₇₁H₁₃₉N₃O₆=1130.1, found [M+H]⁺=1131.3.

### Synthetic Example 25

### Bis(2-Hexyldecyl) 8,8′-((7,7′-(Methylazanediyl)Bis(Heptanoyl))Bis(Hexylazanediyl))Dioctanoate (Compound I-16)

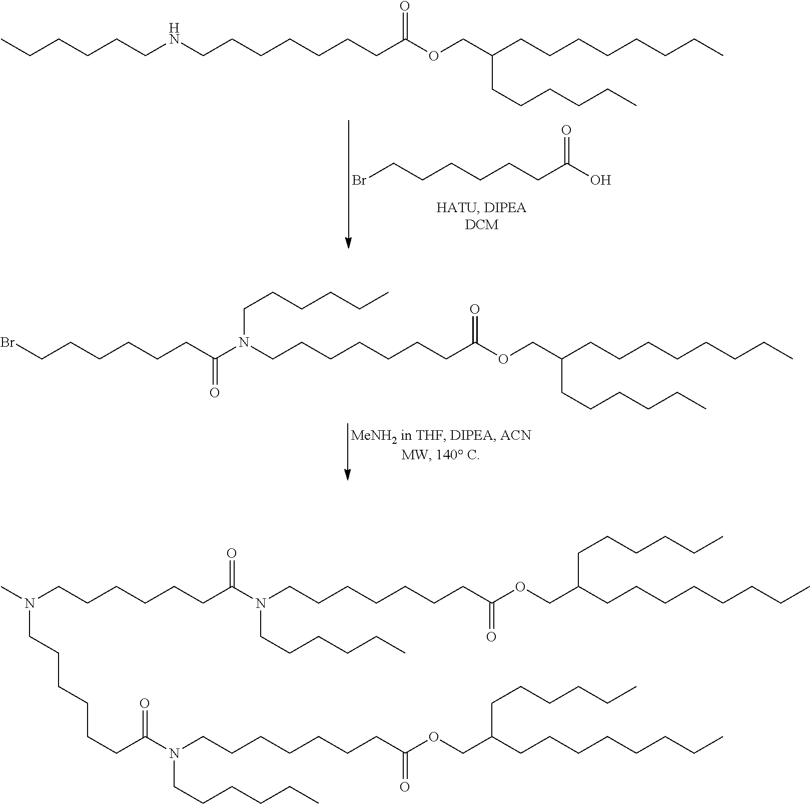

### Synthesis of 2-hexyldecyl 8-(7-bromo-N-hexylheptanamido)octanoate

2-Hexyldecyl 8-(7-bromo-N-hexylheptanamido)octanoate was prepared from 2-hexyldecyl 8-(hexylamino)octanoate (855 μmol, 400 mg), 7-bromoheptanoic acid (941 μmol, 197 mg), DIPEA (1.71 mmol, 221 mg, 299 μL) and HATU (1.11 mmol, 423 mg) by following the procedures outlined herein. Yield (518 mg, 92%). ESI-MS: m/z calculated for C₃₇H₇₂BrNO₃=657.5, found [M+H]⁺=658.7.

### Synthesis of Compound I-16

The title compound was prepared from 2-hexyldecyl 8-(7-bromo-N-hexylheptanamido)octanoate (607 μmol, 400 mg), methyl amine (364 μmol, 2M THF solution, 182 μL) and DIPEA (1.21 mmol, 157 mg, 212 μL) in ACN (0.55 mL), by following the procedures outlined herein. Yield (45 mg, 12%).

¹H NMR (400 MHz, CDCl₃) δ 3.96 (dd, J=5.8, 3.7 Hz, 4H), 3.27 (td, J=7.7, 2.3 Hz, 4H), 3.23-3.14 (m, 4H), 2.34-2.22 (m, 12H), 2.18 (s, 3H), 1.69-1.41 (m, 25H), 1.40-1.22 (m, 82H), 0.94-0.83 (m, 18H). ESI-MS: m/z calculated for C₇₅H₁₄₇N₃O₆=1186.1, found [M+H]⁺=1187.3.

### Synthetic Example 26

### Bis(2-Hexyldecyl) 9,9′-((6,6′-(Methylazanediyl)Bis(Hexanoyl))Bis(Hexylazanediyl))Dinonanoate (Compound I-17)

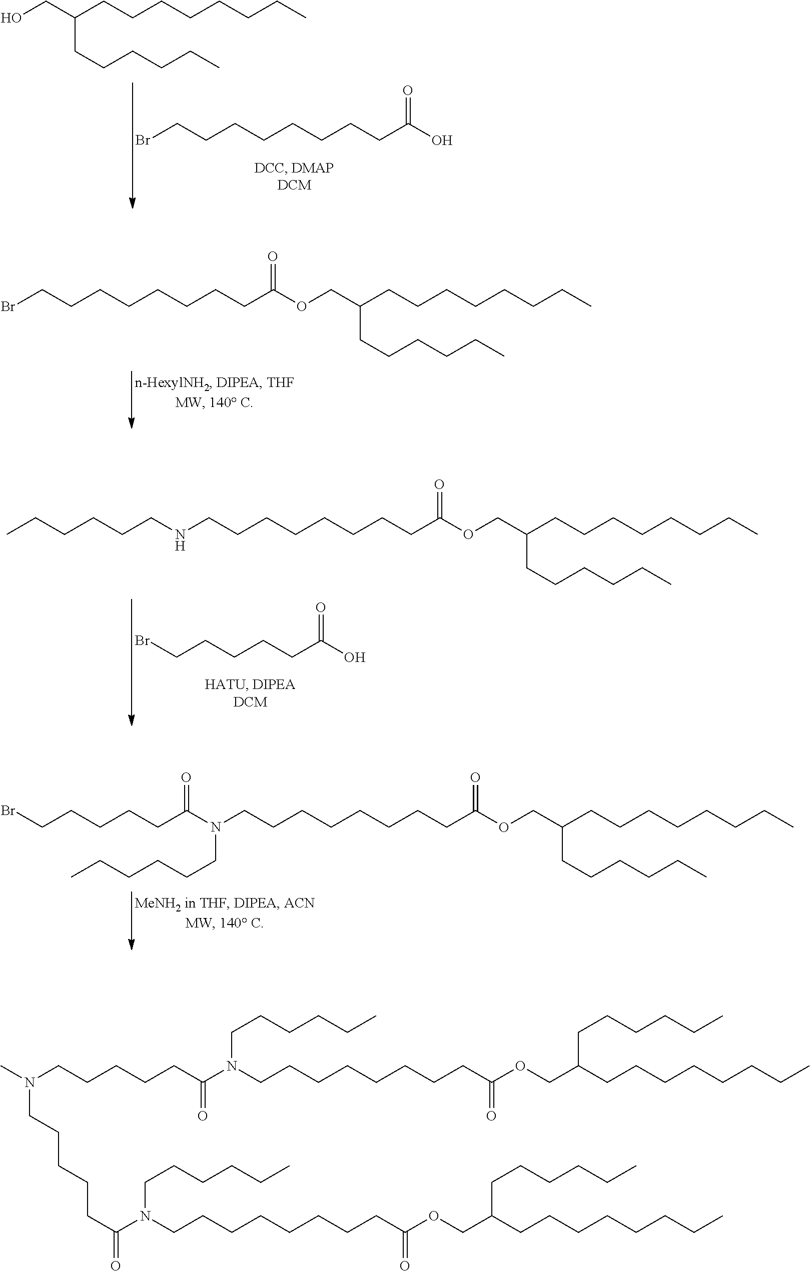

### Synthesis of 2-hexyldecyl 9-bromononanoate

2-Hexyldecyl 9-bromononanoate was prepared from 9-bromononanoic acid (8.43 mmol, 2.00 g), 2-hexyldecan-1-ol (10.1 mmol, 2.45 g, 2.94 mL), DCC (9.28 mmol, 1.91 g) and DMAP (843 μmol, 103 mg) by following the procedures outlined herein. Yield (3.60 g, 82%). ESI-MS: m/z calculated for C₂₅H₄₉BrO₂=460.3, found [M+H]⁺=461.5.

### Synthesis of 2-hexyldecyl 9-(hexylamino)nonanoate

2-Hexyldecyl 9-(hexylamino)nonanoate was prepared from 2-hexyldecyl 9-bromononanoate (3.53 mmol, 1.63 g), hexyl amine (7.1 mmol, 715 μL) and DIPEA (7.1 mmol, 913 μg, 1.23 mL) in ACN (14.1 mL), by following the procedures outlined herein. Yield (900 mg, 53%). ESI-MS: m/z calculated for C₃₁H₆₃NO₂=481.5, found [M+H]⁺=482.7.

### Synthesis of 2-hexyldecyl 9-(6-bromo-N-hexylhexanamido)nonanoate

2-Hexyldecyl 9-(6-bromo-N-hexylhexanamido)nonanoate was prepared from 2-hexyldecyl 9-(hexylamino)nonanoate (830 μmol, 400 mg), 6-bromohexanoic acid (913 μmol, 178 mg), DIPEA (1.66 mmol, 215 mg, 290 μL) and HATU (1.08 mmol, 410 mg) by following the procedures outlined herein. Yield (510 mg, 93%). ESI-MS: m/z calculated for C₃₇H₇₂BrNO₃=657.5, found [M+H]⁺=658.7.

### Synthesis of Compound I-17

The title compound was prepared from 2-hexyldecyl 9-(6-bromo-N-hexylhexanamido)nonanoate (607 μmol, 400 mg), methyl amine (364 μmol, 2M THE solution, 182 μL) and DIPEA (1.21 mmol, 157 mg, 212 μL) in ACN (0.55 mL), by following the procedures outlined herein. Yield (115 mg, 32%).

¹H NMR (400 MHz, CDCl₃) δ 3.96 (dd, J=5.8, 2.6 Hz, 4H), 3.27 (ddd, J=10.1, 6.8, 2.6 Hz, 4H), 3.22-3.14 (m, 4H), 2.35-2.23 (m, 12H), 2.19 (s, 3H), 1.71-1.42 (m, 24H), 1.38-1.19 (m, 82H), 0.94-0.83 (m, 18H). ESI-MS: m/z calculated for C₇₅H₁₄₇N₃O₆=1186.1, found [M+H]⁺=1187.3.

### Synthetic Example 27

### Bis(2-Hexyldecyl) 7,7′-((8,8′-(Methylazanediyl)Bis(Octanoyl))Bis(Hexylazanediyl))Diheptanoate (Compound I-14)

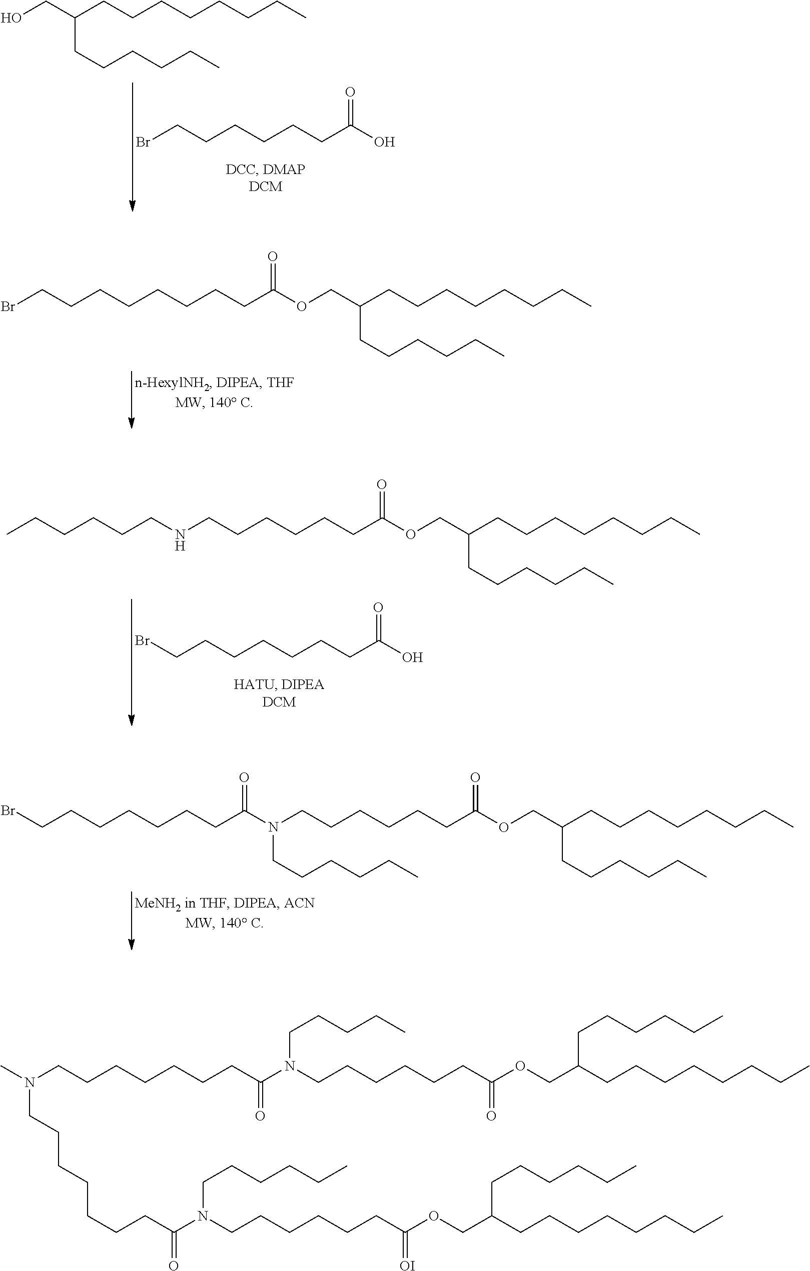

### Synthesis of 2-hexyldecyl 7-bromoheptanoate

2-Hexyldecyl 7-bromoheptanoate was prepared from 7-bromoheptanoic acid (12.0 mmol, 2.50 g), 2-hexyldecan-1-ol (14.3 mmol, 3.48 g, 4.16 mL), DCC (13.2 mmol, 2.71 g) and DMAP (308 μmol, 52 mg) by following the procedures outlined herein. Yield (5.0 g, 96%). ESI-MS: m/z calculated for C₂₃H₄₅BrO₂=432.3, found [M+H]⁺=433.5.

### Synthesis of 2-hexyldecyl 7-(hexylamino)heptanoate

2-Hexyldecyl 7-(hexylamino)heptanoate was prepared from 2-hexyldecyl 7-bromoheptanoate (3.51 mmol, 1.52 g), hexyl amine (7.0 mmol, 710 μL) and DIPEA (7.0 mmol, 908 μg, 1.22 mL) in ACN (14.0 mL), by following the procedures outlined herein. Yield (900 mg, 56%). ESI-MS: m/z calculated for C₃₁H₆₃NO₂=453.5, found [M+H]⁺=454.7.

### Synthesis of 2-hexyldecyl 7-(8-bromo-N-hexyloctanamido)heptanoate

2-Hexyldecyl 7-(8-bromo-N-hexyloctanamido)heptanoate was prepared from 2-hexyldecyl 7-(hexylamino)heptanoate (718 μmol, 326 mg), 8-bromooctanoic acid (790 μmol, 176 mg), DIPEA (1.44 mmol, 186 mg, 251 μL) and HATU (934 μmol, 355 mg) by following the procedures outlined herein. Yield (350 mg, 74%). ESI-MS: m/z calculated for C₃₇H₇₂BrNO₃=657.5, found [M+H]⁺=658.7.

### Synthesis of Compound I-14

The title compound was prepared from 2-hexyldecyl 7-(8-bromo-N-hexyloctanamido)heptanoate (448 μmol, 295 mg), methyl amine (269 μmol, 2M THE solution, 134 μL) and DIPEA (895 μmol, 116 mg, 156 μL) in ACN (0.40 mL), by following the procedures outlined herein. Yield (90 mg, 34%).

¹H NMR (400 MHz, CDCl₃) δ 3.96 (t, J=5.7 Hz, 4H), 3.27 (dd, J=9.0, 6.3 Hz, 4H), 3.22-3.15 (m, 4H), 2.35-2.3 (m, 12H), 2.19 (s, 3H), 1.69-1.39 (m, 29H), 1.39-1.20 (m, 83H), 0.95-0.83 (m, 18H). ESI-MS: m/z calculated for C₇₅H₁₄₇N₃O₆=1186.1, found [M+H]⁺=1187.4.

### Synthetic Example 28

### Bis(2-Hexyldecyl) 5,5′-((8,8′-(Methylazanediyl)Bis(Octanoyl))Bis(Hexylazanediyl))Dipentanoate (Compound I-15)

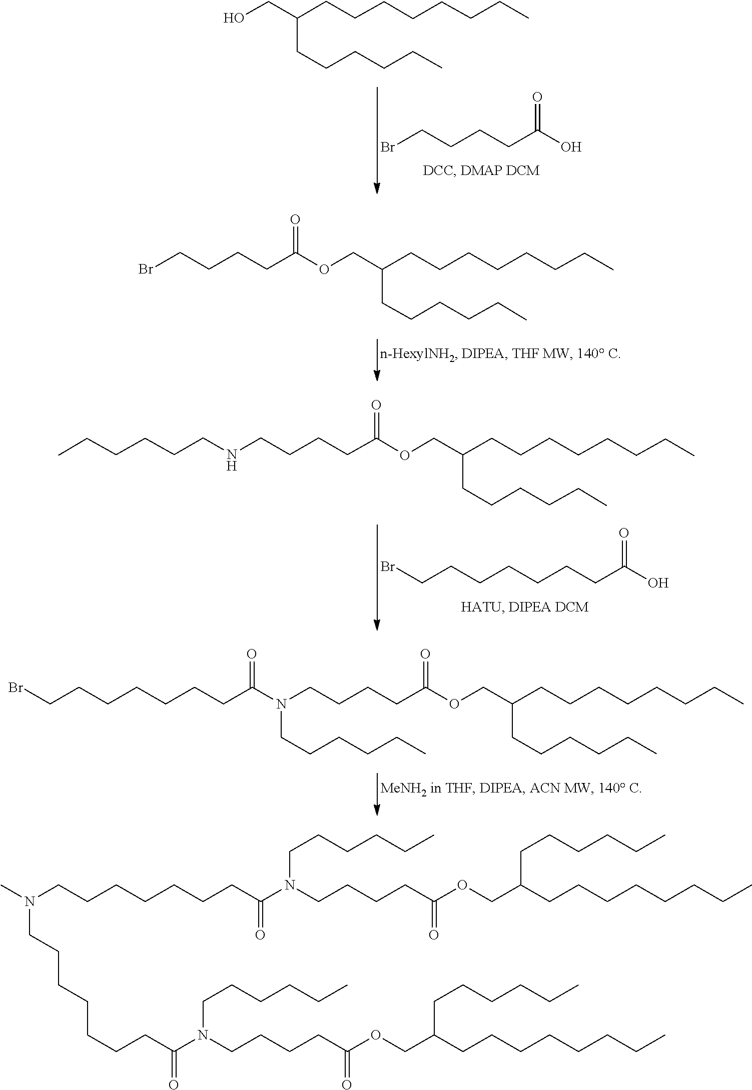

### Synthesis of 2-hexyldecyl 5-bromopentanoate

2-Hexyldecyl 5-bromopentanoate was prepared from 5-bromopentanoic acid (27.6 mmol, 5.0 g), 2-hexyldecan-1-ol (30.9 mmol, 7.5 g, 8.97 mL), DCC (28.4 mmol, 5.85 g) and DMAP (2.58 mmol, 315 mg) by following the procedures outlined herein. Yield (9.5 g, 91%). ESI-MS: m/z calculated for C₂₁H₄₁BrO₂=404.2, found [M+H]⁺=405.4.

### Synthesis of 2-hexyldecyl 7-(hexylamino)heptanoate

2-Hexyldecyl 7-(hexylamino)heptanoate was prepared from 2-hexyldecyl 5-bromopentanoate (3.70 mmol, 1.50 g), hexyl amine (7.40 mmol, 749 μL) and DIPEA (7.4 mmol, 956 μg, 1.29 mL) in ACN (14.8 mL), by following the procedures outlined herein. Yield (900 mg, 58%). ESI-MS: m/z calculated for C₂₇H₅₅NO₂=425.4, found [M+H]⁺=426.7.

### Synthesis of 2-hexyldecyl 5-(8-bromo-N-hexyloctanamido)pentanoate

2-hexyldecyl 5-(8-bromo-N-hexyloctanamido)pentanoate was prepared from 2-hexyldecyl 7-(hexylamino)heptanoate (940 μmol, 400 mg), 8-bromooctanoic acid (940 μmol, 210 mg), DIPEA (1.88 mmol, 243 mg, 328 μL) and HATU (1.22 mmol, 464 mg) by following the procedures outlined herein. Yield (415 mg, 70%). ESI-MS: m/z calculated for C₃₅H₆₈BrNO₃=629.4, found [M+H]⁺=630.6.

### Synthesis of Compound I-15

The title compound was prepared from 2-hexyldecyl 5-(8-bromo-N-hexyloctanamido)pentanoate (607 μmol, 383 mg), methyl amine (364 μmol, 2M THE solution, 182 μL) and DIPEA (1.21 mmol, 157 mg, 212 μL) in ACN (0.54 mL), by following the procedures outlined herein. Yield (91 mg, 27%).

¹H NMR (400 MHz, CDCl₃) δ 3.97 (dd, J=9.0, 5.8 Hz, 4H), 3.34-3.15 (m, 8H), 2.38-2.22 (m, 12H), 2.19 (s, 3H), 1.69-1.39 (m, 26H), 1.38-1.24 (m, 72H), 0.94-0.83 (m, 18H). ESI-MS: m/z calculated for C₇₁H₁₃₉N₃O₆=1130.1, found [M+H]⁺=1131.3.

### Synthetic Example 29

### Bis(2-Hexyldecyl) 3,3′-((8,8′-(Methylazanediyl)Bis(Octanoyl))Bis(Hexylazanediyl))Dipropionate (Compound I-32)

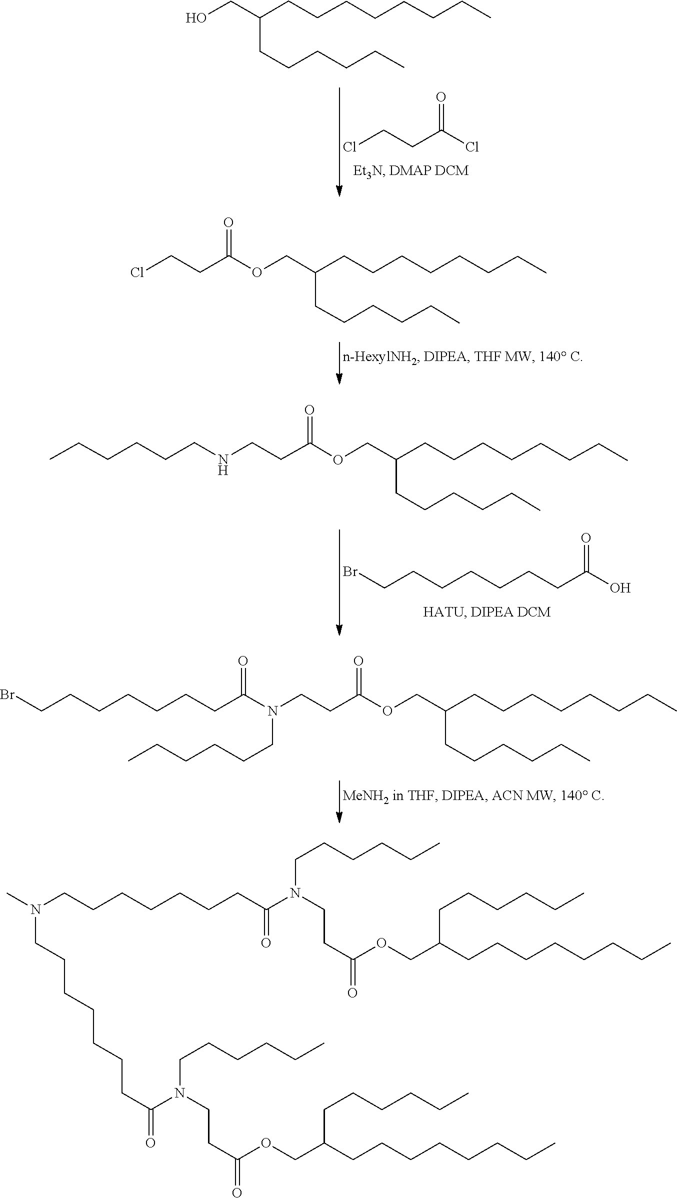

### Synthesis of 2-hexyldecyl 3-chloropropanoate

2-Hexyldecyl 3-chloropropanoate was prepared from commercial 3-chloropropionyl chloride (10.4 mmol, 1M solution in DCM, 10.4 mL), 2-hexyldecan-1-ol (8.66 mmol, 2.1 g), triethyl amine (17.3 mmol, 1.75 g, 2.4 mL) and DMAP (103 μmol, 14 mg) by following the procedures outlined herein. Yield (2.8 g, 97%).

### Synthesis of 2-hexyldecyl 3-(hexylamino)propanoate

2-Hexyldecyl 3-(hexylamino)propanoate was prepared from 2-hexyldecyl 7-bromoheptanoate (3.78 mmol, 1.26 g), hexyl amine (7.57 mmol, 766 μL), DIPEA (7.57 mmol, 978 g, 1.32 mL) and potassium iodide (3.78 mmol, 628 mg) in ACN (14.0 mL), by following the procedures outlined herein. Yield (1.2 g, 80%). ESI-MS: m/z calculated for C₂₅H₅₁NO₂=397.3, found [M+H]⁺=398.6.

### Synthesis of 2-Hexyldecyl 3-(8-bromo-N-hexyloctanamido)propanoate

2-hexyldecyl 3-(8-bromo-N-hexyloctanamido)propanoate was prepared from 2-hexyldecyl 3-(hexylamino)propanoate (1.02 mmol, 405 mg), 8-bromooctanoic acid (1.12 mol, 250 mg), DIPEA (2.04 mmol, 263 mg, 356 μL) and HATU (1.32 mol, 503 mg) by following the procedures outlined herein. Yield (490 mg, 80%). ESI-MS: m/z calculated for C₃₃H₆₄BrNO₃=601.4, found [M+H]⁺=602.6.

### Synthesis of Compound I-32

The title compound was prepared from 2-hexyldecyl 3-(8-bromo-N-hexyloctanamido)propanoate (521 μmol, 314 mg), methyl amine (313 μmol, 2M THF solution, 156 μL) and DIPEA (1.04 mmol, 135 mg, 182 μL) in ACN (0.52 mL), by following the procedures outlined herein. Yield (40 mg, 14%).

¹H NMR (400 MHz, CDCl₃) δ 4.01-3.93 (m, 4H), 3.61-3.51 (m, 4H), 3.33-3.20 (m, 4H), 2.62-2.51 (m, 4H), 2.35-2.22 (m, 8H), 2.19 (s, 3H), 1.68-1.38 (m, 21H), 1.38-1.24 (m, 72H), 0.92-0.82 (t, J=6.7 Hz, 18H). ESI-MS: m/z calculated for C₆₇H₁₃₁N₃O₆=1074.0, found [M+H]⁺=1075.2.

### Synthetic Example 30

### Bis(2-Hexyldecyl) 6,6′-((8,8′-(Methylazanediyl)Bis(Octanoyl))Bis(Decylazanediyl))Dihexanoate (Compound I-40)

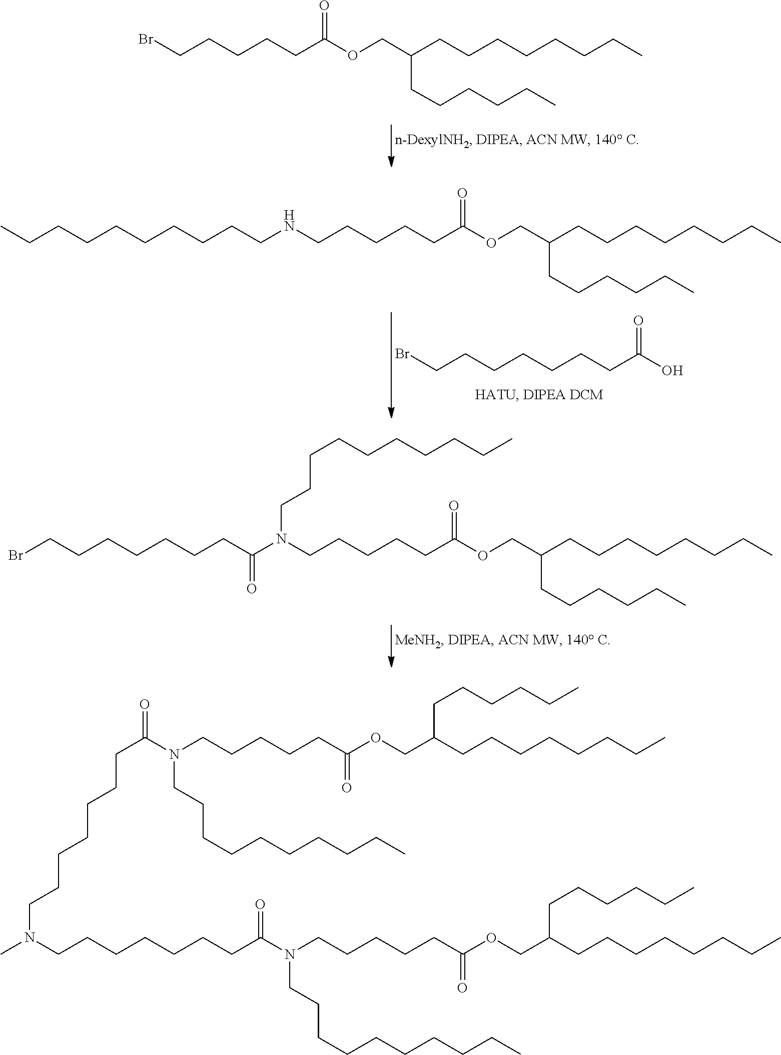

### Synthesis of 2-hexyldecyl 6-(decylamino)hexanoate

2-Hexyldecyl 6-(decylamino)hexanoate was prepared from 2-hexyldecyl 6-bromohexanoate (1.72 mmol, 720 mg), decyl amine (6.89 mmol, 1.08 g) and DIPEA (3.43 mmol, 444 mg, 600 μL) in ACN (13.7 mL,) by following the procedures outlined herein. Yield (499 mg, 59%). ESI-MS: m/z calculated for C₃₂H₆₅NO₂=495.5, found [M+H]⁺=496.7.

### Synthesis of 2-hexyldecyl 6-(8-bromo-N-decyloctanamido)hexanoate

2-hexyldecyl 6-(8-bromo-N-decyloctanamido)hexanoate from 2-hexyldecyl 6-(decylamino)hexanoate (1.01 mmol, 500 mg), 8-bromooctanoic acid (1.11 mmol, 247 mg), DIPEA (2.02 mmol, 262 mg, 352 μL) and HATU (1.31 mmol, 498 mg) by following the procedures outlined herein. Yield (601 mg, 85%). ESI-MS: m/z calculated for C₄₀H₇₈BrNO₃=699.5, found [M+H]⁺=700.7.

### Synthesis of Compound I-40

The title compound was prepared from 2-hexyldecyl 6-(8-bromo-N-decyloctanamido)hexanoate (499 μmol, 350 mg), methyl amine (275 μmol, 2M THE solution, 137 μL) and DIPEA (999 μmol, 129 mg, 174 μL) in ACN (0.42 mL), by following the procedures outlined herein. Yield (100 mg, 32%).

¹H NMR (400 MHz, CDCl₃) δ 3.97 (dd, J=7.9, 5.8 Hz, 4H), 3.32-3.14 (m, 8H), 2.36-2.21 (m, 12H), 2.19 (s, 3H), 1.72-1.38 (m, 28H), 1.36-1.14 (m, 94H), 0.95-0.81 (m, 18H). ESI-MS: m/z calculated for C₈₁H₁₅₉N₃O₆=1270.2, (m+2)/2z calculated for C₈₁H₁₆₁N₃O₆²⁺=636.1, found 636.6.

### Synthetic Example 31

### Bis(2-Hexyldecyl) 6,6′-((8,8′-(Methylazanediyl)Bis(Octanoyl))Bis(Octylazanediyl))Dihexanoate (Compound I-41)

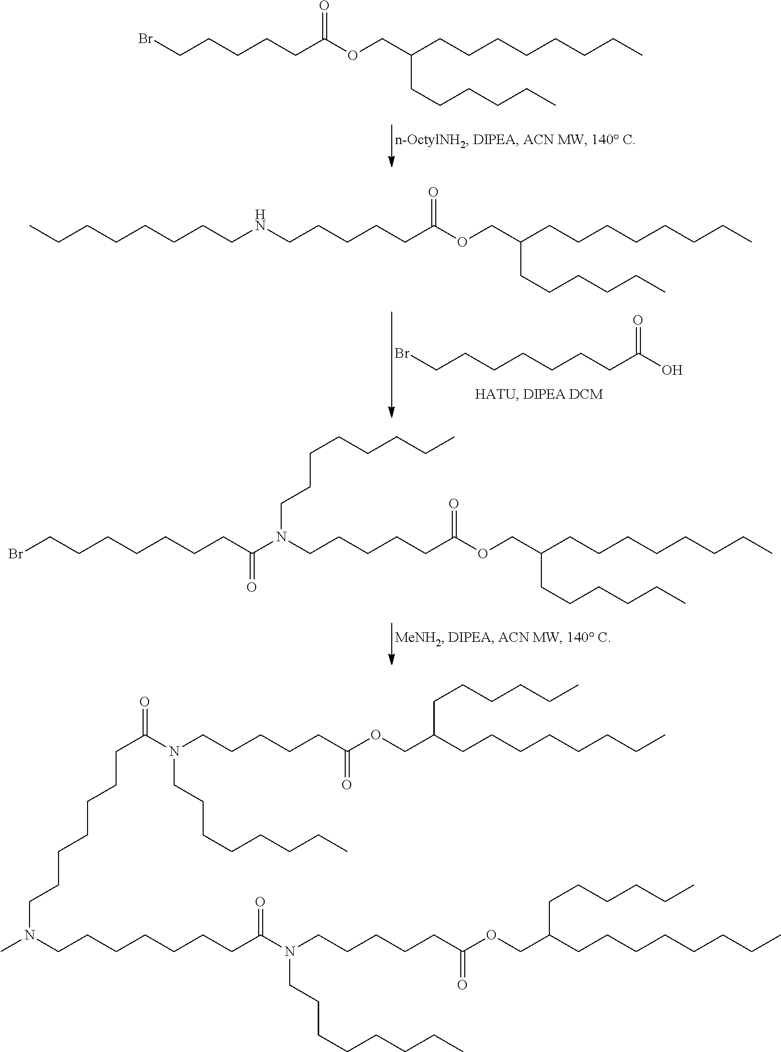

### Synthesis of 2-hexyldecyl 6-(octylamino)hexanoate

2-Hexyldecyl 6-(octylamino)hexanoate was prepared from 2-hexyldecyl 6-bromohexanoate (1.91 mmol, 800 mg), decyl amine (7.63 mmol, 986 mg) and DIPEA (3.81 mmol, 493 mg, 666 μL) in ACN (15.3 mL,) by following the procedures outlined herein. Yield (499 mg, 59%). ESI-MS: m/z calculated for C₃₀H₆₁NO₂=467.5, found [M+H]⁺=468.7.

### Synthesis of 2-hexyldecyl 6-(8-bromo-N-octyloctanamido)hexanoate

2-Hexyldecyl 6-(8-bromo-N-octyloctanamido)hexanoate from 2-hexyldecyl 6-(octylamino)hexanoate (1.39 mmol, 650 mg), 8-bromooctanoic acid (1.53 mmol, 341 mg), DIPEA (2.78 mmol, 359 mg, 485 μL) and HATU (1.81 mmol, 687 mg) by following the procedures outlined herein. Yield (750 mg, 80%). ESI-MS: m/z calculated for C₃₈H₇₄BrNO₃=671.5, found [M+H]⁺=672.7.

### Synthesis of Compound I-41

The title compound was prepared from 2-hexyldecyl 6-(8-bromo-N-octyloctanamido)hexanoate (520 μmol, 350 mg), methyl amine (286 μmol, 2M THE solution, 143 μL) and DIPEA (1.04 mmol, 134 mg, 182 μL) in ACN (0.44 mL), by following the procedures outlined herein. Yield (65 mg, 21%).

¹H NMR (400 MHz, CDCl₃) δ 3.97 (dd, J=7.9, 5.8 Hz, 4H), 3.32-3.14 (m, 8H), 2.41-2.23 (m, 12H), 2.19 (s, 3H), 1.72-1.38 (m, 28H), 1.38-1.17 (m, 87H), 0.92-0.83 (m, 18H). ESI-MS: m/z calculated for C₈₁H₁₅₉N₃O₆=1214.2, found [M+H]⁺=1215.3.

### Synthetic Example 32

### Bis(2-Butyloctyl) 6,6′-((8,8′-(Methylazanediyl)Bis(Octanoyl))Bis(Hexylazanediyl))Dihexanoate (Compound I-33)

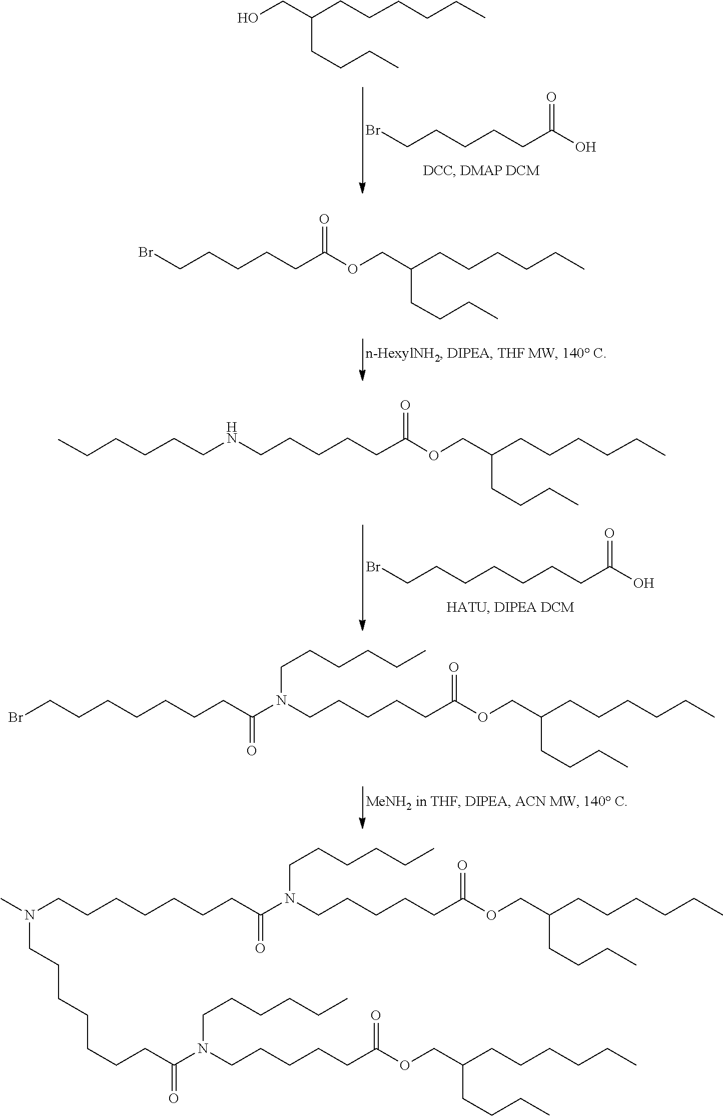

### Synthesis of 2-butyloctyl 6-bromohexanoate

2-Butyloctyl 6-bromohexanoate was prepared from 6-bromohexanoic acid (5.13 mmol, 1.0 g), 2-butyloctan-1-ol (6.66 mmol, 1.24 g, 1.49 mL), DCC (6.15 mmol, 1.25 g) and DMAP (513 mol, 63 mg) by following the procedures outlined herein. Yield (1.55 g, 83%). ESI-MS: m/z calculated for C₁₈H₃₅BrO₂=362.2, found [M+H]⁺=363.4.

### Synthesis of 2-butyloctyl 6-(hexylamino)hexanoate

2-Butyloctyl 6-(hexylamino)hexanoate was prepared from 2-butyloctyl 6-bromohexanoate (3.34 mmol, 1.21 g), hexyl amine (8.34 mmol, 844 μL) and DIPEA (6.67 mmol, 862 μg, 1.17 mL) in ACN (16.7 mL), by following the procedures outlined herein. Yield (900 mg, 70%). ESI-MS: m/z calculated for C₂₄H₄₉NO₂=383.4, found [M+H]⁺=384.7.

### Synthesis of 2-butyloctyl 6-((8-bromooctyl)(hexyl)amino)hexanoate

2-Butyloctyl 6-((8-bromooctyl)(hexyl)amino)hexanoate was prepared from 2-butyloctyl 6-(hexylamino)hexanoate (1.05 mmol, 402 mg), 8-bromooctanoic acid (1.15 mmol, 257 mg), DIPEA (2.10 mmol, 271 mg, 366 μL) and HATU (1.36 mmol, 518 mg) by following the procedures outlined herein. Yield (533 mg, 86%). ESI-MS: m/z calculated for C₃₂H₆₄BrNO₃=587.4, found [M+H]⁺=588.6.

### Synthesis of Compound I-33

The title compound was prepared from 2-butyloctyl 6-((8-bromooctyl)(hexyl)amino)hexanoate (510 μmol, 300 mg), methyl amine (306 μmol, 2M THF solution, 153 μL) and DIPEA (1.02 mmol, 132 mg, 178 μL) in ACN (0.30 mL), by following the procedures outlined herein. Yield (65 mg, 24%).

¹H NMR (400 MHz, CDCl₃) δ 3.97 (dd, J=7.8, 5.8 Hz, 4H), 3.32-3.14 (m, 8H), 2.36-2.21 (m, 12H), 2.19 (s, 3H), 1.78-1.40 (m, 30H), 1.40-1.19 (m, 62H), 0.93-0.83 (m, 18H). ESI-MS: m/z calculated for C₆₅H₁₂₇N₃O₆=1045.9, found [M+H]⁺=1047.1.

### Synthetic Example 33

### Bis(2-Ethylhexyl) 6,6′-((8,8′-(Methylazanediyl)Bis(Octanoyl))Bis(Hexylazanediyl))Dihexanoate (Compound I-42)

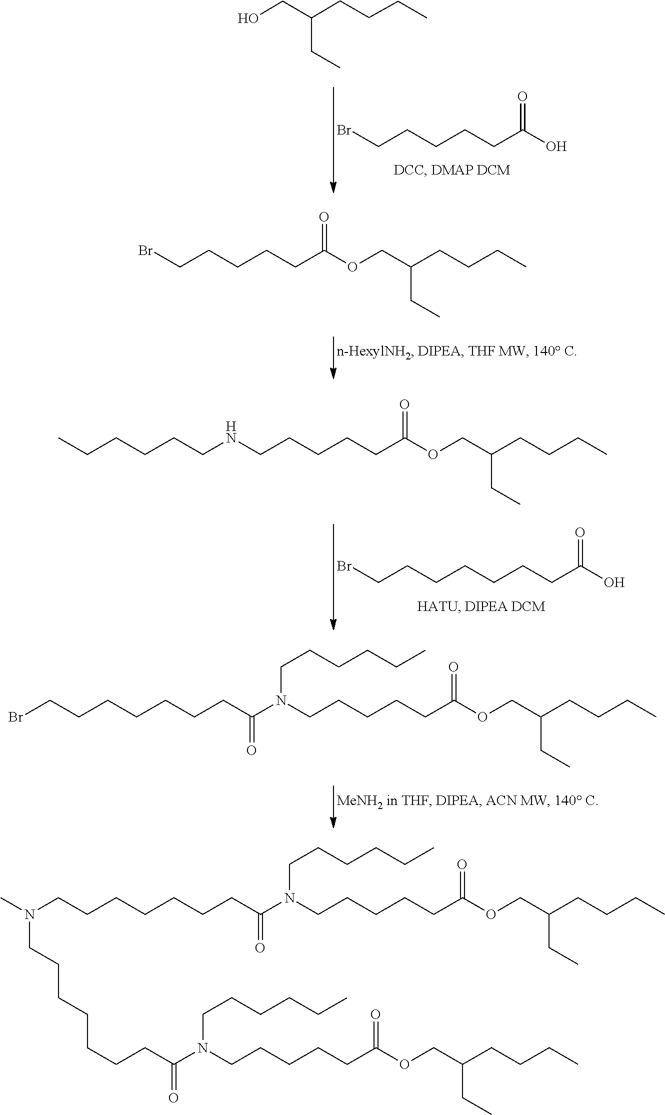

### Synthesis of 2-ethylhexyl 6-bromohexanoate

2-Ethylhexyl 6-bromohexanoate was prepared from 6-bromohexanoic acid (5.13 mmol, 1.0 g), 2-ehylhexan-1-ol (6.66 mmol, 1.04 g, 868 μL), DCC (6.15 mmol, 1.25 g) and DMAP (513 μmol, 63 mg) by following the procedures outlined herein. Yield (1.48 g, 94%).

### Synthesis of 2-ethylhexyl 6-(hexylamino)hexanoate

2-Ethylhexyl 6-(hexylamino)hexanoate was prepared from 2-ethylhexyl 6-bromohexanoate (3.34 mmol, 1.02 g), hexyl amine (8.34 mmol, 844 μL) and DIPEA (6.67 mmol, 862 μg, 1.17 mL) in ACN (16.7 mL), by following the procedures outlined herein. Yield (730 mg, 67%). ESI-MS: m/z calculated for C₂₀H₄₁NO₂=327.3, found [M+H]⁺=328.3.

### Synthesis 2-ethylhexyl 6-((8-bromooctyl)(hexyl)amino)hexanoate

2-Ethylhexyl 6-((8-bromooctyl)(hexyl)amino)hexanoate was prepared from 2-ethylhexyl 6-(hexylamino)hexanoate (2.23 mmol, 730 mg), 8-bromooctanoic acid (2.45 mmol, 547 mg), DIPEA (4.46 mmol, 576 mg, 779 μL) and HATU (2.90 mmol, 1.1 g) by following the procedures outlined herein. Yield (903 mg, 76%). ESI-MS: m/z calculated for C₂₈H₅₆BrNO₃=531.3, found [M+H]⁺=532.4.

### Synthesis of Compound I-42

The title compound was prepared from 2-ethylhexyl 6-((8-bromooctyl)(hexyl)amino)hexanoate (657 μmol, 350 mg), methyl amine (427 μmol, 2M THF solution, 214 μL) and DIPEA (1.31 mmol, 170 mg, 230 μL) in ACN (0.70 mL), by following the procedures outlined herein. Yield (40 mg, 13%).

¹H NMR (400 MHz, CDCl₃) δ 4.04-3.92 (m, 4H), 3.32-3.14 (m, 8H), 2.36-2.21 (m, 12H), 2.19 (s, 3H), 1.72-1.43 (m, 25H), 1.40-1.17 (m, 46H), 0.95-0.83 (m, 18H). ESI-MS: m/z calculated for C₅₇H₁₁N₃O₆=933.8, found [M+H]⁺=935.0.

### Synthetic Example 34

### (((Methylazanediyl)Bis(Hexane-6,1-Diyl))Bis(Butyrylazanediyl))Bis(Octane-8,1-Diyl) Bis(2-Hexyldecanoate) (Compound I-47)

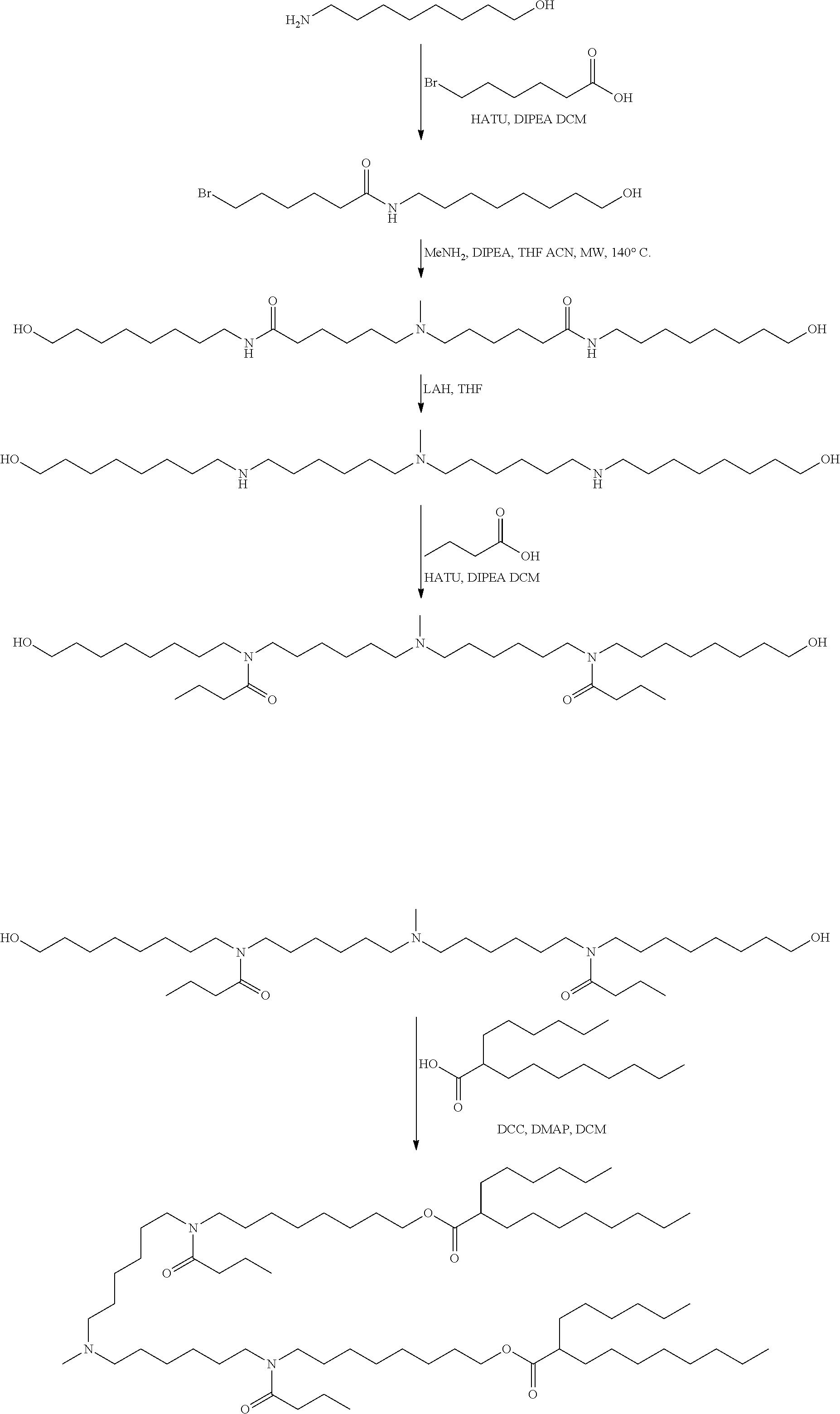

### Synthesis 6-bromo-N-(8-hydroxyoctyl)hexanamide

6-Bromo-N-(8-hydroxyoctyl)hexanamide was prepared from 8-amino-1-octanol (11.3 mmol, 1.64 g), 6-bromohexanoic acid (10.3 mmol, 2.00 g), DIPEA (20.50 mmol, 2.65 g, 3.58 mL) and HATU (13.3 mmol, 5.07 g) by following the procedures outlined herein. Yield (3.0 g, 91%). ESI-MS: m/z calculated for C₁₄H₂₈BrNO₂=321.1, found [M+H]⁺=322.3.

### Synthesis of 6,6′-(methylazanediyl)bis(N-(8-hydroxyoctyl)hexanamide)

6,6′-(Methylazanediyl)bis(N-(8-hydroxyoctyl)hexanamide) was prepared from 6-bromo-N-(8-hydroxyoctyl)hexanamide (3.01 mmol, 970 mg), methyl amine (1.66 mmol, 8M EtOH solution, 207 μL) and DIPEA (6.02 mmol, 778 mg, 1.05 mL) in ACN (15.2 mL), by following the procedures outlined herein. Yield (390 mg, 50%). ESI-MS: m/z calculated for C₂₉H₅₉N₃O₄=513.5, found [M+H]⁺=514.6.

### Synthesis of 8,8′-(((methylazanediyl)bis(hexane-6,1-diyl))bis(azanediyl))bis(octan-1-ol)

8,8′-(((methylazanediyl)bis(hexane-6,1-diyl))bis(azanediyl))bis(octan-1-ol) was prepared from 6,6′-(methylazanediyl)bis(N-(8-hydroxyoctyl)hexanamide) (2.28 mmol, 1.17 g), and LAH (11.8 mmol, 446 mg) by following the procedures outlined herein. Yield (550 mg, 50%). ESI-MS: m/z calculated for C₂₉H₆₃N₃O₂=485.5, found [M+H]⁺=486.7.

### Synthesis N,N-((methylazanediyl)bis(hexane-6,1-diyl))bis(N-(8-hydroxyoctyl)butyramide)

N,N-((methylazanediyl)bis(hexane-6,1-diyl))bis(N-(8-hydroxyoctyl)butyramide) was prepared from 8,8′-(((methylazanediyl)bis(hexane-6,1-diyl))bis(azanediyl))bis(octan-1-ol) (1.13 mmol, 550 mg), butyric acid (2.83 mmol, 249 mg), DIPEA (4.53 mmol, 585 mg, 791 μL) and HATU (3.17 mmol, 1.21 g) by following the procedures outlined herein. Yield (85 mg, 12%). ESI-MS: m/z calculated for C₃₇H₇₅N₃O₄=625.6, found [M+H]⁺=626.8.

### Synthesis of Compound I-47

The title compound was prepared from N,N-((methylazanediyl)bis(hexane-6,1-diyl))bis(N-(8-hydroxyoctyl)butyramide) (112 μmol, 70 mg), 2-hexyldecanoic acid (257 μmol, 65.9 mg, 75.5 L), DCC (291 mmol, 60 mg) and DMAP (34 μmol, 4 mg) by following the procedures outlined herein. Yield (50 mg, 41%)

¹H NMR (400 MHz, CDCl₃) δ 4.09-4.00 (m, 4H), 3.33-3.24 (m, 4H), 3.22-3.12 (m, 4H), 2.36-2.15 (m, 13H), 1.81-1.38 (m, 33H), 1.38-1.16 (m, 66H), 0.95 (t, J=7.4 Hz, 6H), 0.88 (p, J=6.6 Hz, 12H). ESI-MS: m/z calculated for C₆₉H₁₃₅N₃O₆=1102.0, found [M+H]⁺=1103.2.

### Synthetic Example 35

### 2-Hexyldecyl 6-(8-((8-(Hexyl(6-((2-Hexyldecyl)Oxy)-6-Oxohexyl)Amino)-8-Oxooctyl)(Methyl)Amino)Octanamido)Hexanoate (Compound I-11)

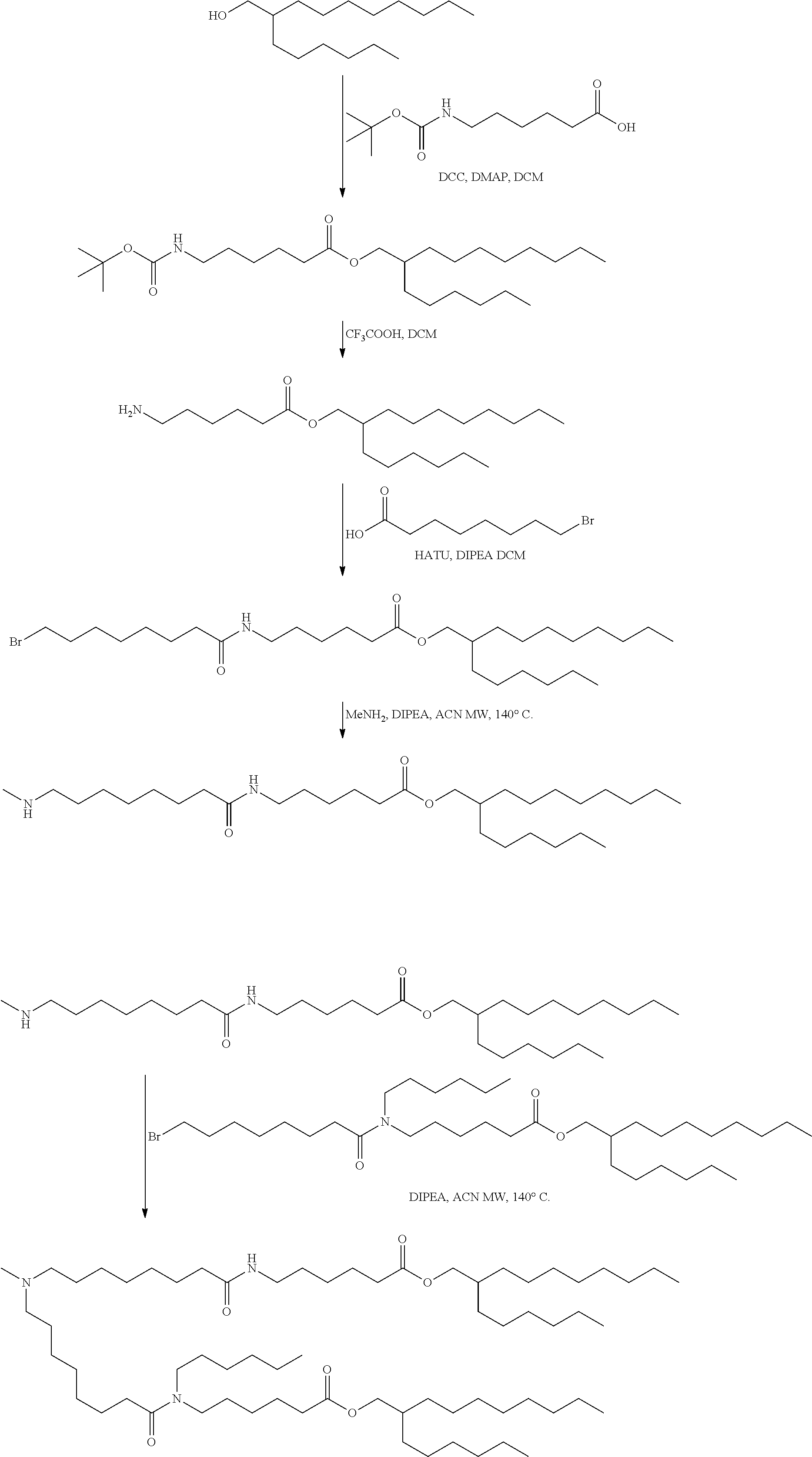

### Synthesis of 2-hexyldecyl 6-((tert-butoxycarbonyl)amino)hexanoate

2-Hexyldecyl 6-((tert-butoxycarbonyl)amino)hexanoate was prepared from commercial Boc-protected 6-aminohexanoic acid (10.8 mmol, 2.50 g), 2-hexyl-1-decanol (13.0 mmol, 3.14 g), DCC (13.0 mmol, 2.68 g) and DMAP (2.16 mol, 264 mg) by following the procedures outlined herein. Yield (4.1 g, 83%).

### Synthesis of 2-hexyldecyl 6-aminohexanoate

To 2-hexyldecyl 6-((tert-butoxycarbonyl)amino)hexanoate (4.39 mmol, 2.00 g) in DCM (20 ml) was added trifluoroacetic acid (65.8 mmol, 7.51 g, 5.04 mL). The reaction mixture was stirred at room temperature over night. After the addition of 10 mL of water, reaction mixture was neutralized with solid NaHCO₃ and pH adjusted (pH stripes) to 12 by addition of 1M aqueous KOH. Phases were separated and aqueous layer extracted 3 times with 10 mL DCM. Combined organic phase was dried over anhydrous sodium sulfate, filtered and the solvent removed under vacuum to give crude 2-hexyldecyl 6-aminohexanoate (1.3 g, 83%) which was used in the next step without further purification. ESI-MS: m/z calculated for C₂₂H₄₅NO₂=355.3, found [M+H]⁺=356.5.

### Synthesis 2-hexyldecyl 6-(8-bromooctanamido)hexanoate

2-Hexyldecyl 6-(8-bromooctanamido)hexanoate was prepared from 2-hexyldecyl 6-aminohexanoate (3.09 mmol, 1.10 g), 8-bromooctanoic acid (3.40 mmol, 759 mg), DIPEA (6.19 mmol, 800 mg, 1.08 mL) and HATU (4.02 mmol, 1.53 g) by following the procedures outlined herein. Yield (953 mg, 55%). ESI-MS: m/z calculated for C₃₀H₅₈BrNO₃=559.4, found [M+H]⁺=560.5.

### Synthesis of 2-hexyldecyl 6-(8-(methylamino)octanamido)hexanoate

2-Hexyldecyl 6-(8-(methylamino)octanamido)hexanoate was prepared from 2-hexyldecyl 6-(8-bromooctanamido)hexanoate (1.47 mmol, 823 mg), methyl amine (4.40 mmol, 8M solution in EtOH, 550 μL) and DIPEA (2.94 mmol, 379 g, 513 mL) in ACN (5.0 mL), by following the procedures outlined herein. Yield (550 mg, 73%). ESI-MS: m/z calculated for C₃₁H₆₂N₂O₃=510.5, found [M+H]⁺=511.6.

### Synthesis of Compound I-11

The title compound was prepared from 2-ethylhexyl 6-bromohexanoate (540 μmol, 276 mg), 2-hexyldecyl 6-(8-bromo-N-hexyloctanamido)hexanoate (270 μmol, 174 mL) and DIPEA (51.2 μmol, 69.7 μg, 94.3 μL) in ACN (1.1 mL), by following the procedures outlined herein. Yield (55 mg, 19%).

¹H NMR (400 MHz, CDCl₃) δ 4.00-3.92 (m, 4H), 3.32-3.14 (m, 6H), 2.35-2.20 (m, 10H), 2.22-2.07 (m, 5H), 1.72-1.17 (m, 98H), 0.94-0.83 (m, 15H). ESI-MS: m/z calculated for C₆₇H₁₃₁N₃O₆=1074.0, found [M+H]⁺=1075.2.

### Synthetic Example 36

### Bis(2-Hexyldecyl) 6,6′-(((Methylazanediyl)Bis(Octane-8,1-Diyl))Bis(Hexanoylazanediyl))Dihexanoate (Compound I-43)

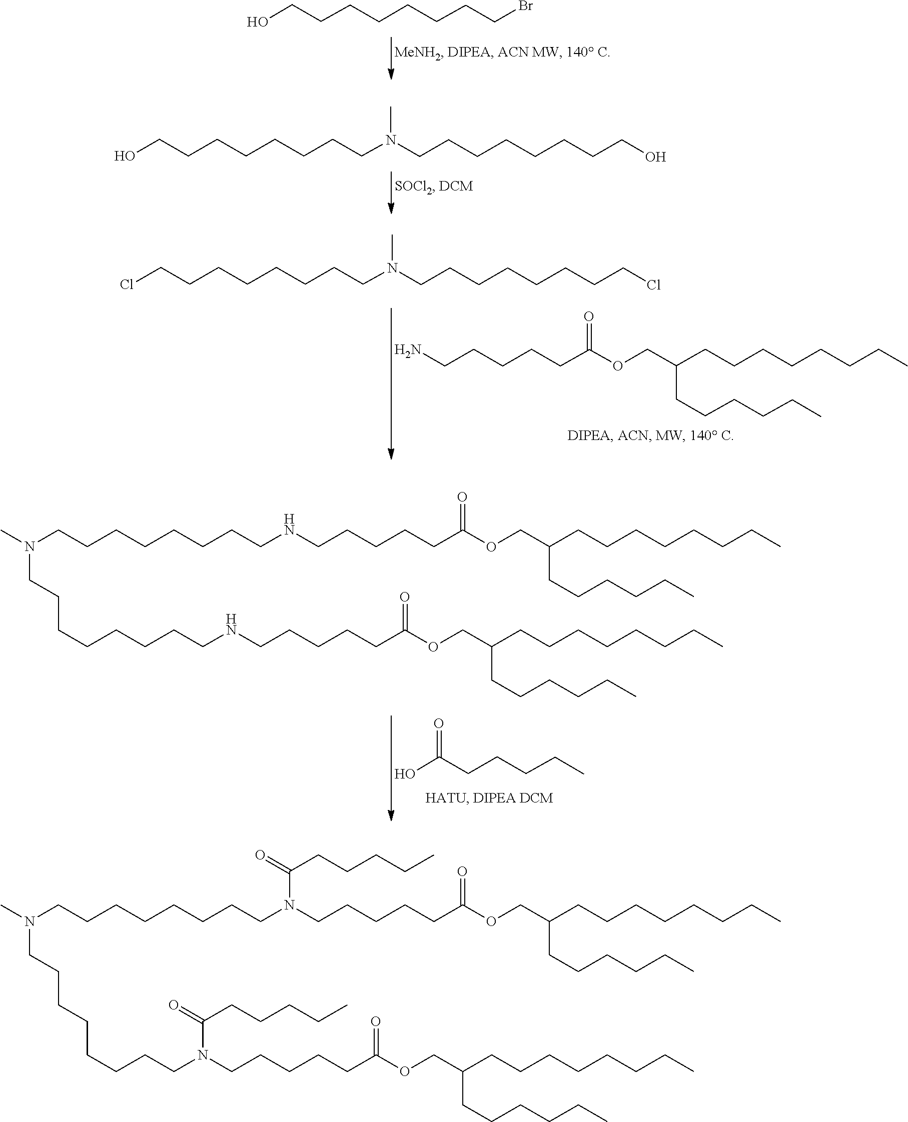

### Synthesis of 8,8′-(methylazanediyl)bis(octan-1-ol)

8,8′-(Methylazanediyl)bis(octan-1-ol) was prepared from 8-bromo-1-octanol (19.1 mmol, 4.0 g), methyl amine (10.5 mmol, 8M EtOH solution, 1.3 mL) and DIPEA (38.2 mmol, 4.93 g, 6.7 mL) in ACN (8.0 mL), by following the procedures outlined herein. Yield (1.5 g, 36%). ESI-MS: m/z calculated for C₂₉H₅₉N₃O₄=287.3, found [M+H]⁺=288.3.

### Synthesis of 8-chloro-N-(8-chlorooctyl)-N-methyloctan-1

To 8,8′-(methylazanediyl)bis(octan-1-ol) (3.48 mmol, 1.00 g) in DCM (30 mL), SOCl₂ (24.8 mmol, 4.11 g, 2.52 mL) added. Reaction mixture was stirred over night at room temperature. The solvent and the excess thionyl chloride were removed under vacuum. The residue was taken in hexanes containing 1% Et3N; white precipitate formed and was removed by filtration. The filtrate was concentrated and purified via flash chromatography (0% to 10% MeOH in chloroform) to give 8-chloro-N-(8-chlorooctyl)-N-methyloctan-1-amine (1.1 g, 97%). ESI-MS: m/z calculated for C₁₇H₃₅C₂N=323.2, found [M+H]⁺=324.3.

### Synthesis of bis(2-hexyldecyl) 6,6′-(((methylazanediyl)bis(octane-8,1-diyl))bis(azanediyl))dihexanoate

bis(2-hexyldecyl) 6,6′-(((methylazanediyl)bis(octane-8,1-diyl))bis(azanediyl))dihexanoate was prepared from 8-chloro-N-(8-chlorooctyl)-N-methyloctan-1-amine (1.54 mmol, 500 mg), 2-hexyldecyl 6-aminohexanoate (6.17 mmol, 2.19 g) and DIPEA (6.17 mmol, 797 mg, 1.08 mL) in ACN (5.0 mL), by following the procedures outlined herein. Yield (230 mg, 16%). ESI-MS: m/z calculated for C₆₁H₁₂₃N₃O₄=962.0, found [M+H]⁺=963.1.

### Synthesis Compound I-43

The title compound was prepared from bis(2-hexyldecyl) 6,6′-(((methylazanediyl)bis(octane-8,1-diyl))bis(azanediyl))dihexanoate (177 μmol, 170 mg), hexanoic acid (389 μml, 45 mg), DIPEA (706 μmol, 91 mg, 123 μL) and HATU (459 μmol, 175 mg) by following the procedures outlined herein. Yield (150 mg, 73%).

¹H NMR (400 MHz, CDCl₃) δ 4.0-3.91 (m, 4H), 3.32-3.14 (m, 8H), 2.39-2.22 (m, 12H), 2.19 (s, 3H), 1.74-1.40 (m, 27H), 1.40-1.15 (m, 77H), 0.93-0.83 (m, 18H). ESI-MS: m/z calculated for C₇₃H₁₄₃N₃O₆=1158.1, found [M+H]⁺=1159.2.

### Synthetic Example 37

### Bis(2-Hexyldecyl) 6,6′-(((Methylazanediyl)Bis(Octane-8,1-Diyl))Bis(Decanoylazanediyl))Dihexanoate (Compound I-44)

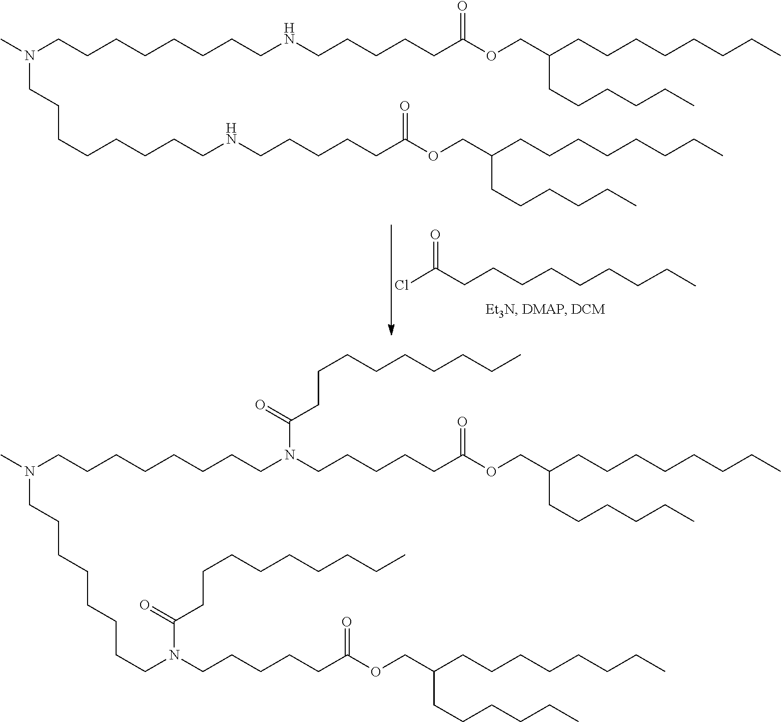

### Synthesis Compound I-44

The title compound was prepared from bis(2-hexyldecyl) 6,6′-(((methylazanediyl)bis(octane-8,1-diyl))bis(azanediyl))dihexanoate (62 μmol, 60 mg), commercial decanoyl chloride (156 μmol, 30 mg, 32 μL), DMAP (18 μmol, 2 mg) and triethyl amine (436 μmol, 44 mg, 61 μL) by following the procedures outlined herein. Yield (54 mg, 68%).

¹H NMR (400 MHz, CDCl₃) δ 4.0-3.91 (m, 4H), 3.32-3.14 (m, 8H), 2.45-2.23 (m, 12H), 2.20 (s, 3H), 1.75-1.39 (m, 30H), 1.39-1.11 (m, 95H), 0.93-0.83 (m, 18H). ESI-MS: m/z calculated for C₈₁H₁₅₉N₃O₆=1270.3, (m+2)/2z calculated for C₈₁H₁₆₁N₃O₆²⁺=636.1, found 636.6.

### Synthetic Example 38

### ((8,8′-((2-(Dimethylamino)Ethyl)Azanediyl)Bis(Octanoyl))Bis(Hexylazanediyl))Bis(Hexane-6,1-Diyl) Bis(2-Hexyldecanoate) (Compound I-34)

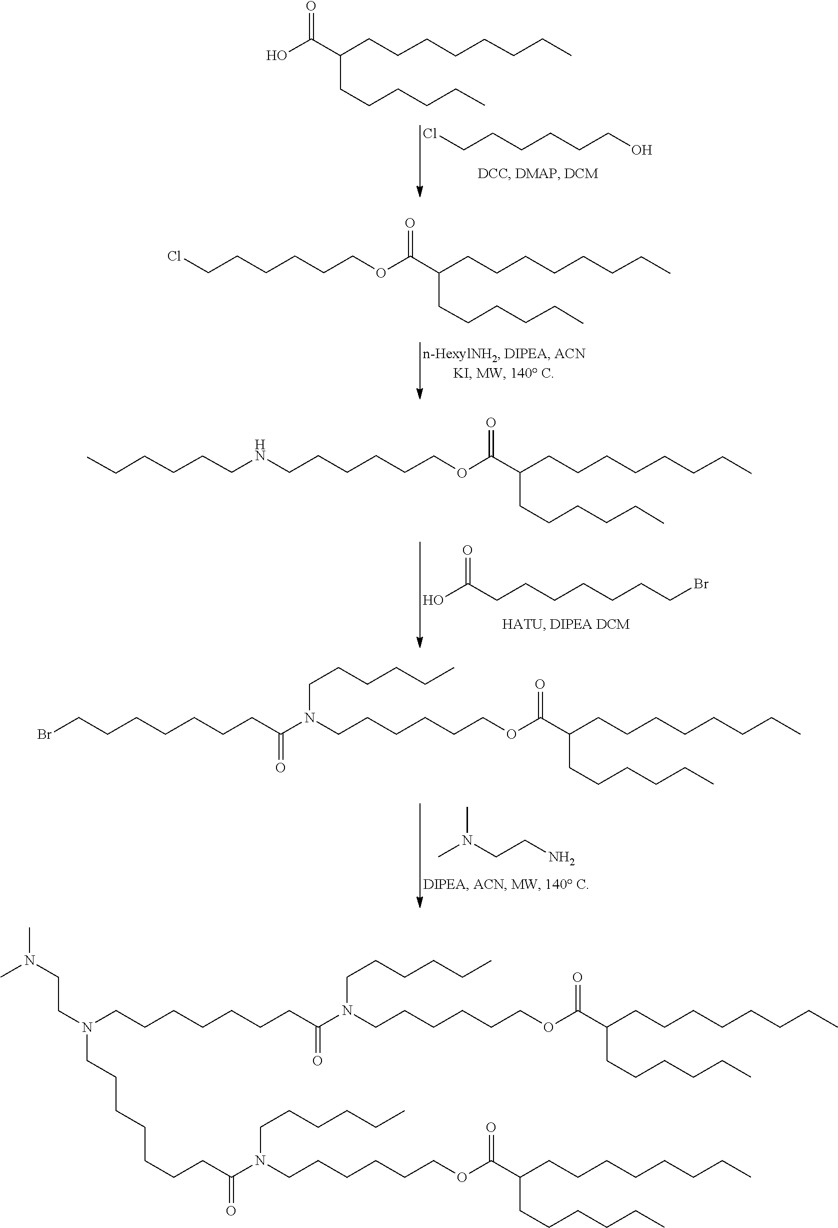

### Synthesis of 6-chlorohexyl 2-hexyldecanoate

6-Chlorohexyl 2-hexyldecanoate was prepared from 2-hexyldecanoic acid (40.3 mmol, 10.3 g), 6-chloro-1-hexanol (36.6 mmol, 5.0 g), DMAP (3.6 mmol, 439 mg) and DCC (43.9 mmol, 9.1 g) by following the procedures outlined herein. Yield (12.2 g, 89%). ESI-MS: m/z calculated for C₂₂H₄₃ClO₂=374.3, found [M+H]⁺=375.5.

### Synthesis of 6-(hexylamino)hexyl 2-hexyldecanoate

6-(Hexylamino)hexyl 2-hexyldecanoate was prepared from 6-chlorohexyl 2-hexyldecanoate (4.00 mmol, 1.50 g), 1-hexylamine (8.00 mmol, 809 mg), potassium iodide (4.00 mmol, 664 mg) and DIPEA (8.00 mmol, 1.03 g, 1.4 mL) in ACN (16.0 mL), by following the procedures outlined herein. Yield (1.45 g, 82%). ESI-MS: m/z calculated for C₂₈H₅₇NO₂=439.4, found [M+H]⁺=440.7.

### Synthesis 6-(8-bromo-N-hexyloctanamido)hexyl 2-hexyldecanoate

6-(8-Bromo-N-hexyloctanamido)hexyl 2-hexyldecanoate was prepared from 6-(hexylamino)hexyl 2-hexyldecanoate (1.13 mmol, 498 mg), 8-bromooctanoic acid (1.36 mmol, 303 mg), DIPEA (2.26 mmol, 293 mg, 395 μL) and HATU (1.47 mmol, 559 mg) by following the procedures outlined herein. Yield (521 mg, 71%). ESI-MS: m/z calculated for C₃₆H₇₀BrNO₃=643.4, found [M+H]⁺=644.7.

### Synthesis of Compound I-34

The title compound was prepared from 6-(8-bromo-N-hexyloctanamido)hexyl 2-hexyldecanoate (444 μmol, 286 mg), N,N-dimethylethane-1,2-diamine (244 μmol, 22 mg, 27 μL) and DIPEA (887 μmol, 115 mg, 155 μL) in ACN (0.49 mL), by following the procedures outlined herein. Yield (45 mg, 17%).

¹H NMR (400 MHz, CDCl₃) δ 4.09-4.00 (m, 4H), 3.31-3.24 (m, 4H), 3.23-3.15 (m, 4H), 2.70-2.20 (m, 19H), 1.68-1.14 (m, 107H), 0.93-0.80 (m, 18H). ESI-MS: m/z calculated for C₇₆H₁₅₀N₄O₆=1215.2, found [M+H]⁺=1216.3.

### Synthetic Example 39

### 7,23,32-Trihexyl-16-Methyl-8,22,29-Trioxo-30-Oxa-7,16,23-Triazatetracontyl 2-Hexyldecanoate (Compound I-46)

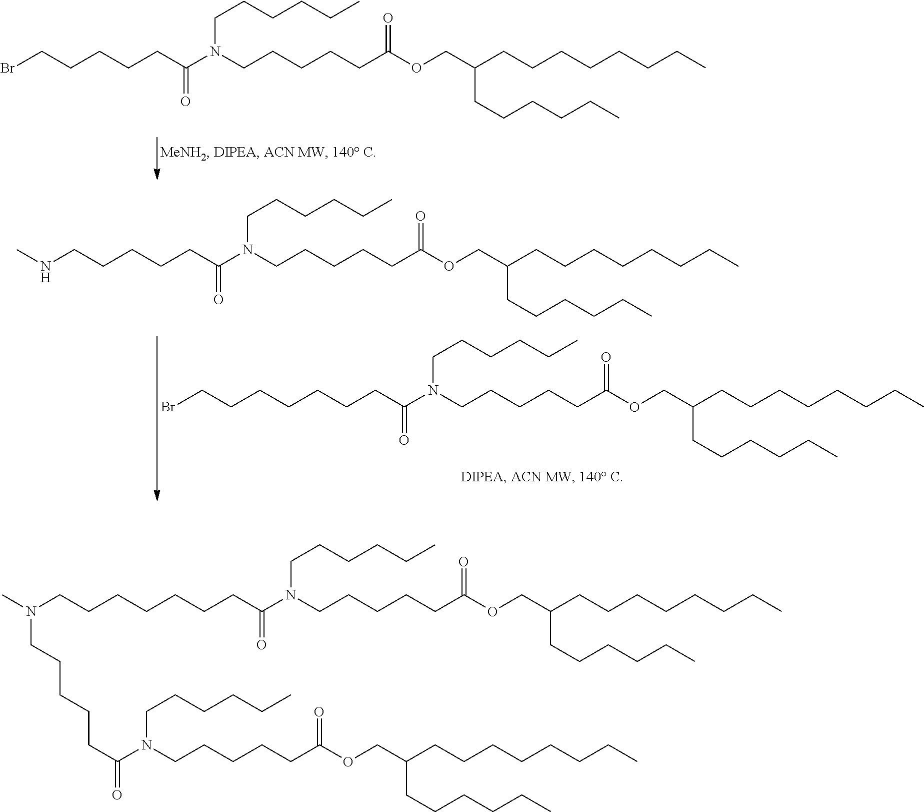

### Synthesis of 2-hexyldecyl 6-(N-hexyl-6-(methylamino)hexanamido)hexanoate

2-hexyldecyl 6-(N-hexyl-6-(methylamino)hexanamido)hexanoate was prepared from 2-hexyldecyl 6-(6-bromo-N-hexylhexanamido)hexanoate (1.55 mmol, 956 mg) and methylamine (10.1 mmol, 2M THF solution, 5.04 mL) in ACN (15 mL), by following the procedures outlined herein. Yield (710 mg, 81%). ESI-MS: m/z calculated for C₃₅H₇₀N₂O₃=566.5, found [M+H]⁺=567.7.

### Synthesis of Compound I-46

The title compound was prepared from 6-(8-bromo-N-hexyloctanamido)hexyl 2-hexyldecanoate (171 μmol, 110 mg), 2-hexyldecyl 6-(N-hexyl-6-(methylamino)hexanamido)hexanoate (341 μmol, 203 mL) and DIPEA (341 μmol, 40 mg, 60 μL) in ACN (0.68 mL), by following the procedures outlined herein. Yield (65 mg, 34%).

4.06 (dt, J=8.9, 6.6 Hz, 2H), 3.96 (dd, J=7.9, 5.8 Hz, 2H), 3.32-3.14 (m, 8H), 2.36-2.22 (m, 11H), 2.18 (s, 3H), 1.74-1.37 (m, 28H), 1.37-1.16 (m, 67H), 0.94-0.83 (td, 18H). ESI-MS: m/z calculated for C₇₁H₁₃₉N₃O₆=1130.1, found [M+H]⁺=1131.2.

### Synthetic Example 40

### (((methylazanediyl)bis(hexane-6,1-diyl))bis(hexylazanediyl))bis(9-oxononane-9,1-diyl) bis(2-hexyldecanoate) (Compound I-13)

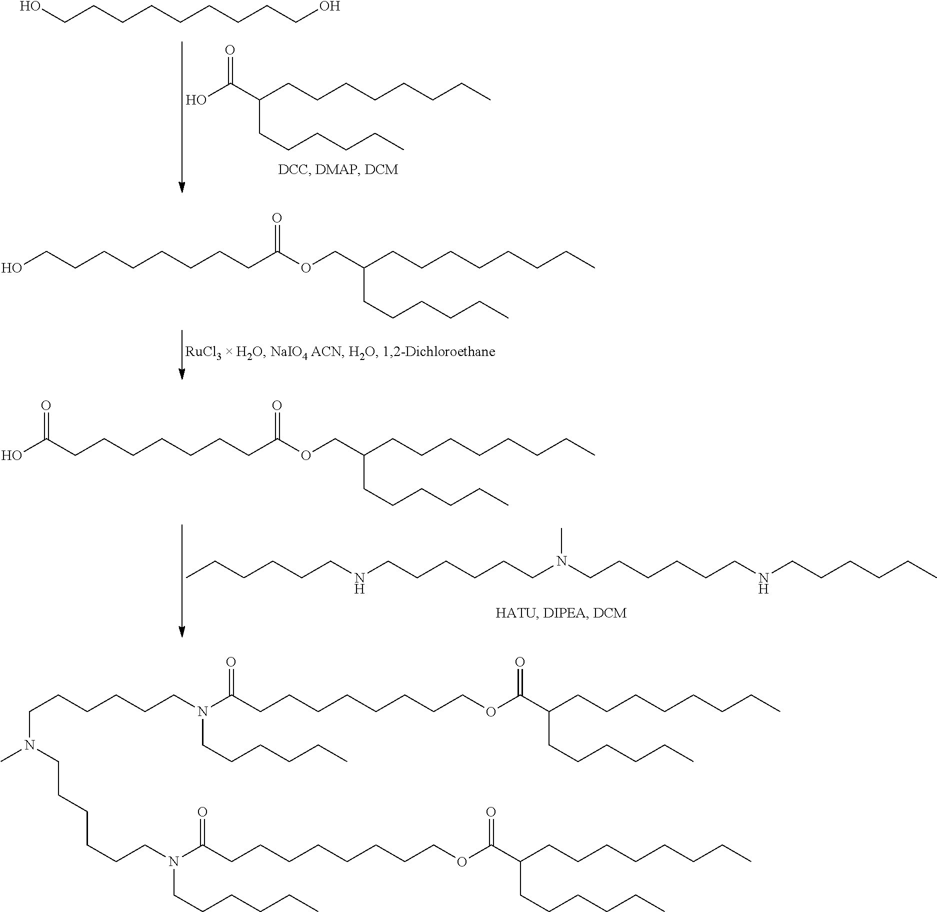

### Synthesis of 2-hexyldecyl 9-hydroxynonanoate

2-Hexyldecyl 9-hydroxynonanoate was prepared from 2-hexyldecanoic acid (31.8 mmol, 8.14 g), 1,9-nonadiol (31.8 mmol, 5.09 g), DMAP (953 μmol, 116 mg) and DCC (31.8 mmol, 6.55 g) by following the procedures outlined herein. Yield (5.8 g, 46%). ESI-MS: m/z calculated for C₂₅H₅₀O₃=398.4, found [M+H]⁺=399.6.

### Synthesis of 9-((2-hexyldecyl)oxy)-9-oxononanoic acid

To 2-hexyldecyl 9-hydroxynonanoate (2.51 mmol, 1.00 g) in a mixture of water (5.28 mL), ACN (3.96 mL) and 1,2-dichloroethane (3.96 min) were added RuCl₃×H₂O (125 μmol, 28 mg) and NaIO₄ (7.52 mmol, 1.61 g) and reaction was stirred at room temperature for 15 min. The mixture was passed through a small pad of Celite, diluted with EtOAc and dried over anhydrous MgSO₄. Purification via flash chromatography (0% to 10% MeOH in DCM) give 9-((2-hexyldecyl)oxy)-9-oxononanoic acid (950 mg, 92%). ESI-MS: m/z calculated for C₂₅H₄₈O₄=412.4, found [M+H]⁺=413.6.

### Synthesis of Compound I-13

The title compound was prepared from 9-((2-hexyldecyl)oxy)-9-oxononanoic acid (553 μmol, 228 mg), 14-methyl-7,14,21-triazaheptacosane (251 μmol, 100 mg), DIPEA (1.01 mmol, 130 mg, 176 μL) and HATU (704 μmol, 268 mg) by following the procedures outlined herein. Yield (105 mg, 35%).

¹H NMR (400 MHz, CDCl₃) δ 4.09-4.01 (m, 4H), 3.41-3.24 (m, 4H), 3.24-3.10 (m, 4H), 2.36-2.22 (m, 10H), 2.22-2.16 (m, 3H), 1.74-1.38 (m, 35H), 1.38-1.18 (m, 80H), 0.95-0.83 (m, 18H). ESI-MS: m/z calculated for C₇₅H₁₄₇N₃O₆=1186.1, found [M+H]⁺=1187.4.

### Synthetic Example 41

### Bis(2-Hexyldecyl) 6,6′-(((Methylazanediyl)Bis(Octane-8,1-Diyl))Bis(Hexylazanediyl))Bis(6-Oxohexanoate) (Compound I-45)

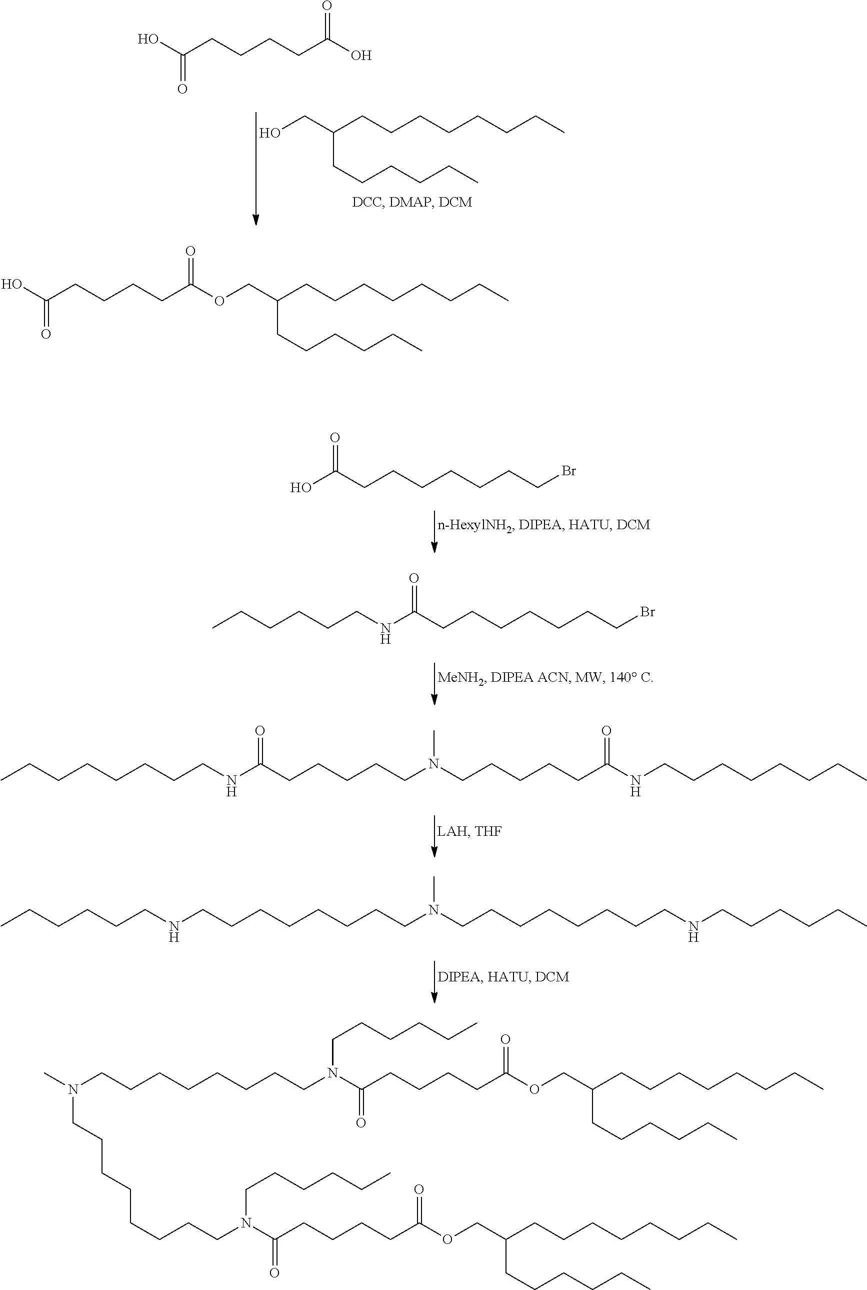

### Synthesis of 6-((2-hexyldecyl)oxy)-6-oxohexanoic acid

6-((2-Hexyldecyl)oxy)-6-oxohexanoic acid was prepared from 2-hexyl-1-decanol (12.4 mmol, 3.00 g), adipic acid (24.7 mmol, 3.62 g), DMAP (124 μmol, 15 mg) and DCC (16.1 mmol, 3.32 g) by following the procedures outlined herein. Yield (1.5 g, 33%). ESI-MS: m/z calculated for C₂₂H_{4204=370.3}, found [M+H]⁺=371.5.

### Synthesis of 8-bromo-N-hexyloctanamide

8-Bromo-N-hexyloctanamide was prepared from 1-hexylamine (17.5 mmol, 1.77 g, 2.31 mL), 8-bromooctanoic acid (13.4 mmol, 3.00 g), DIPEA (26.9 mmol, 3.48 g, 4.7 mL) and HATU (17.5 mmol, 6.65 g) by following the procedures outlined herein. Yield (3.0 g, 73%). ESI-MS: m/z calculated for C₁₄H₂₈BrNO=305.1, found [M+H]⁺=306.2.

### Synthesis of 8,8′-(methylazanediyl)bis(N-hexyloctanamide)

8,8′-(Methylazanediyl)bis(N-hexyloctanamide) was prepared from 8-bromo-N-hexyloctanamide (8.10 mmol, 2.48 g), methylamine (4.45 mmol, 2M THF solution, 2.23 mL) and DIPEA (16.20 mmol, 2.09 g, 2.83 mL) in ACN (6.7 mL), by following the procedures outlined herein. Yield (620 mg, 32%). ESI-MS: m/z calculated for C₂₉H₅₉N₃O₂=481.5, found [M+H]⁺=482.6.

### Synthesis of 16-methyl-7, 16, 25-triazahentriacontane

16-Methyl-7, 16, 25-triazahentriacontane was prepared from 8,8′-(methylazanediyl)bis(N-hexyloctanamide) (1.25 mmol, 600 mg), and LAH (4.98 mmol, 189 mg) by following the procedures outlined herein. Yield (450 mg, 80%). ESI-MS: m/z calculated for C₂₉H₆₃N₃=453.5, found [M+H]⁺=454.6.

### Synthesis of Compound I-45

The title compound was prepared from 16-methyl-7, 16, 25-triazahentriacontane (209 μmol, 95 mg), 6-((2-hexyldecyl)oxy)-6-oxohexanoic acid (461 μmol, 171 mg), DIPEA (1.05 mmol, 135 mg, 183 μL) and HATU (544 μmol, 207 mg) by following the procedures outlined herein. Yield (70 mg, 29%).

¹H NMR (400 MHz, CDCl₃) δ 3.96 (d, J=5.8 Hz, 4H), 3.30-3.24 (m, 4H), 3.21-3.15 (m, 4H), 2.37-2.25 (m, 12H), 2.19 (bs, 3H), 1.70-1.63 (m, 9H), 1.63-1.38 (m, 19H), 1.37-1.19 (m, 79H), 0.94-0.83 (m, 18H). ESI-MS: m/z calculated for C₇₃H₁₄₃N₃O₆=1158.1, found [M+H]⁺=1159.3.

### Synthetic Example 42

### 2-Hexyldecyl 6-((3-((8-((6-((2-Butyloctyl)Oxy)-6-Oxohexyl)(Decyl)Amino)-8-Oxooctyl)(Methyl)Amino)Propyl)(Hexadecyl)Amino)-6-Oxohexanoate (Compound I-38)

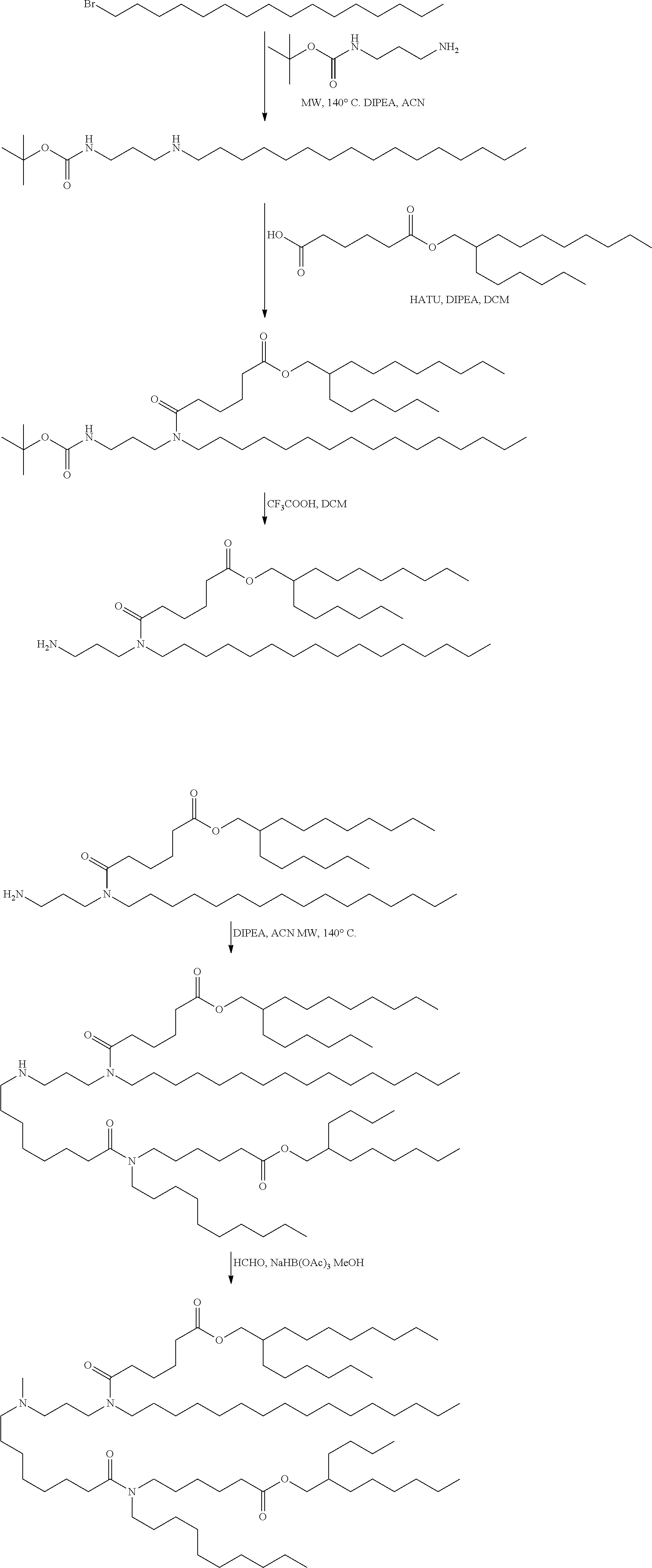

### Synthesis of tert-butyl (3-(hexadecylamino)propyl)carbamate

tert-Butyl (3-(hexadecylamino)propyl)carbamate was prepared from tert-butyl (3-aminopropyl)carbamate (7.86 mmol, 1.37 g), 1-bromohexadecane (3.93 mmol, 1.20 g) and DIPEA (7.86 mmol, 1.02 g, 1.37 mL) in ACN (15.7 mL), by following the procedures outlined herein. Yield (830 mg, 53%). ESI-MS: m/z calculated for C₂₄H₅₀N₂O₂=398.4, found [M+H]⁺=399.6.

### Synthesis of 2-hexyldecyl 6-((3-((tert-butoxycarbonyl)amino)propyl)(hexadecyl)amino)-6-oxohexanoate

2-hexyldecyl 6-((3-((tert-butoxycarbonyl)amino)propyl)(hexadecyl)amino)-6-oxohexanoate was prepared from tert-butyl (3-(hexadecylamino)propyl)carbamate (1.15 mmol, 460 mg), 6-((2-hexyldecyl)oxy)-6-oxohexanoic acid (432 μmol, 117 mg), DIPEA (2.31 mmol, 298 mg, 403 μL) and HATU (1.5 mmol, 570 mg) by following the procedures outlined herein. Yield (595 mg, 69%). ESI-MS: m/z calculated for C₄₆H₉₀rN₂O₅=750.7, found [M+H]⁺=751.8.

### Synthesis of 2-Hexyldecyl 6-((3-aminopropyl)(hexadecyl)amino)-6-oxohexanoate

2-Hexyldecyl 6-((3-aminopropyl)(hexadecyl)amino)-6-oxohexanoate was prepared from 2-hexyldecyl 6-((3-((tert-butoxycarbonyl)amino)propyl)(hexadecyl)amino)-6-oxohexanoate (779 μmol, 585 mg), by following the procedures outlined herein. Yield (460 mg, 91%). ESI-MS: m/z calculated for C₄₁H₈₂N₂O₃=650.6, found [M+H]⁺=651.8.

### Synthesis of 2-hexyldecyl 6-((3-((8-((6-((2-butyloctyl)oxy)-6-oxohexyl)(decyl)amino)-8-oxooctyl)amino)propyl)(hexadecyl)amino)-6-oxohexanoate

2-hexyldecyl 6-((3-((8-((6-((2-butyloctyl)oxy)-6-oxohexyl)(decyl)amino)-8-oxooctyl)amino)propyl)(hexadecyl)amino)-6-oxohexanoate was prepared from 2-hexyldecyl 6-(8-bromo-N-decyloctanamido)hexanoate (205 μmol, 132 mg), 2-hexyldecyl 6-((3-aminopropyl)(hexadecyl)amino)-6-oxohexanoate (409 μmol, 267 mg) and DIPEA (409 mmol, 53 mg, 72 μL) in ACN (0.82 mL), by following the procedures outlined herein. Yield (144 mg, 58%). ESI-MS: m/z calculated for C₇₇H₁₅₁N₃O₆=1214.2, found [M+H]⁺=1215.3.

### Synthesis of Compound I-48

A solution of 2-hexyldecyl 6-((3-((8-((6-((2-butyloctyl)oxy)-6-oxohexyl)(decyl)amino)-8-oxooctyl)amino)propyl)(hexadecyl)amino)-6-oxohexanoate (119 μmol, 144 mg) and formaldehyde (1.48 mmol, 37% water solution, 120 μL) in MeOH (1 mL) was stirred for 10 min at room temperature and then sodium triacetoxyborohydride (356 μmol, 75 mg) was added. The reaction mixture was stirred at room temperature for 36 h, during which time four additional portions of sodium triacetoxyborohydride (356 μmol, 75 mg each) were added. The mixture was diluted with DCM, washed with saturated aqueous sodium bicarbonate solution, dried over anhydrous sodium sulfate, and concentrated. Purification via flash chromatography (0% to 80% EtOAc in Hexanes spiked with 1% Et₃N) gave the title compound (35 mg, 24%).

¹H NMR (400 MHz, CDCl₃) δ 4.01-3.92 (m, 4H), 3.35-3.21 (m, 4H), 3.23-3.11 (m, 4H), 2.36-2.22 (m, 12H), 2.18 (d, J=1.7 Hz, 3H), 1.85-1.39 (m, 23H), 1.38-1.16 (m, 91H), 0.93-0.84 (m, 18H). ESI-MS: m/z calculated for C₇₈H₁₅₃N₃O₆=1228.2, found [M+H]⁺=1229.4.

The various embodiments described above can be combined to provide further embodiments. All the U.S. patents, U.S. patent application publications, U.S. patent applications, foreign patents, foreign patent applications and non-patent publications referred to in this specification are incorporated herein by reference, in their entirety. Aspects of the embodiments can be modified, if necessary to employ concepts of the various patents, applications, and publications to provide yet further embodiments.

These and other changes can be made to the embodiments considering the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1. A compound having a structure of Formula (I): 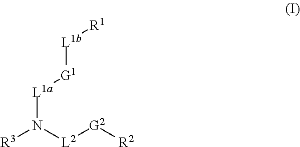
or a pharmaceutically acceptable salt, tautomer, or stereoisomer thereof, wherein:
G¹ is —NR^{1a}—C(═O)—, —C(═O)—NR^{1b}—, or —N(C(═O)R^{1d})—;
G² is —NR^{2a}—C(═O)—, —C(═O)—NR^{2b}—, or —N(C(═O)R^{2e})—;
R¹ is —O—C(═O)R^{1c} or —C(═O)—OR^{1c};
R² is C₁-C₁₂ alkyl, C₂-C₁₂ alkenyl, C₂-C₁₂ alkynyl, or -L^{2a}-R^{2c};
R^{1a}, R¹¹, R^{2a}, and R^{2b} are each independently hydrogen, C₁-C₁₆ alkyl, C₂-C₁₂ alkenyl, or C₂-C₁₂ alkynyl;
R^{1c} is C₆-C₂₄ alkyl, C₆-C₂₄ alkenyl, or C₆-C₂₄ alkynyl;
R^{2c} is —O—C(═O)R^{2d} or —C(═O)—OR^{2d};
R^{2d} is C₁-C₂₄ alkyl, C₂-C₂₄ alkenyl, or C₂-C₂₄ alkynyl;
R^{1d} and R^{2e} are each independently C₁-C₁₂ alkyl;
R³ is C₁-C₈ alkyl or C₃-C₈ cycloalkyl, each of which is optionally substituted with one or more substituents selected from the group consisting of C₁-C₄ alkoxy, oxo, —OH, —N(R^{3a})R^{3b}, cycloalkyl, heterocyclyl, or heteroaryl;
R^{3a} and R^{3b} are each independently hydrogen, C₁-C₄ alkyl, or C₃-C₈ cycloalkyl;
L^{1a} and L² are each independently C₂-C₁₂ alkylene;
L^{1b} is C₁-C₁₂ alkylene, C₂-C₁₂ alkenylene, or C₂-C₁₂ alkynylene; and
L^{2a} is C₁-C₁₂ alkylene, C₂-C₁₂ alkenylene, or C₂-C₁₂ alkynylene,
wherein each alkyl, alkenyl, alkynyl, alkylene, alkenylene, alkynylene, cycloalkyl, heterocyclyl and heteroaryl is optionally substituted with one or more fluoro.

2. The compound of any one of claim 1, wherein G¹ is —NR^{1a}—C(═O)— and R^{1a} is hydrogen.

3-4. (canceled)

5. The compound of claim 2, wherein R^{1a} is unsubstituted n-hexyl.

6. The compound of claim 1, wherein G¹ is —C(═O)—NR^{1b}— and R^{1b} is hydrogen or C₁-C₆ alkyl.

7-8. (canceled)

9. The compound of claim 6, wherein R^{1b} is unsubstituted methyl, unsubstituted ethyl, unsubstituted isopropyl, or unsubstituted n-hexyl.

10. The compound of claim 1, wherein R¹ is —O—C(═O)R^{1c} or —C(═O)—OR^{1c} and R^{1c} is branched and unsubstituted C₆-C₂₄ alkyl.

11-13. (canceled)

14. The compound of claim 1, wherein R^{1c} is branched and unsubstituted C₈-C₁₈ alkyl.

15. The compound of claim 1, wherein G¹ is —N(C(═O)R^{1d})— and R^{1d} is unbranched and unsubstituted C₃, C₅, or C₉ alkyl.

16. The compound of claim 1, wherein R¹ has one of the following structures:

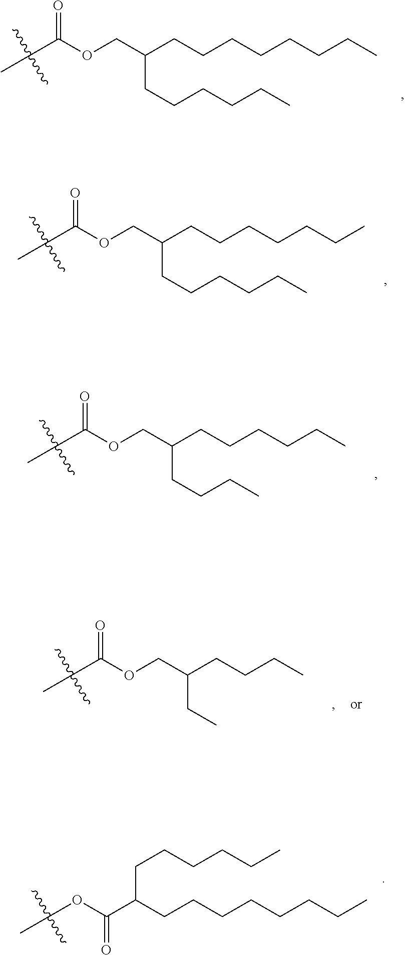 [SMILES: *C(=O)OCC(CCCCCC)CCCCCCCC ; *C(=O)OCC(CCCCCC)CCCCCCC ; *C(=O)OCC(CCCC)CCCCCC ; *C(=O)OCC(CC)CCCC ; *OC(=O)C(CCCCCC)CCCCCCCC]

17. The compound of claim 1, wherein G² is —NR^{2a}—C(═O)— and R^{2a} is hydrogen or C₁-C₆ alkyl.

18-19. (canceled)

20. The compound of claim 17, wherein R^{2a} is unsubstituted n-hexyl.

21. (canceled)

22. The compound of claim 1, wherein G² is —C(═O)—NR^{2b}— and R^{2b} is hydrogen or C₁-C₆ alkyl.

23-26. (canceled)

27. The compound of claim 22, wherein R^{2b} is unsubstituted methyl, unsubstituted ethyl, unsubstituted isopropyl, unsubstituted n-hexyl, unbranched and unsubstituted C₁₀ alkyl or unsubstituted and unbranched C₁₆ alkyl.

28-30. (canceled)

31. The compound of claim 1, wherein R² is C₁-C₁₂ alkyl or -L^{2a}-R^{2c}.

32-33. (canceled)

34. The compound of claim 1, wherein R² is unbranched and unsubstituted C₈-C₁₂ alkyl.

35-37. (canceled)

38. The compound of claim 1, wherein L²a is unsubstituted C₁ or C₂ alkylene or unbranched and unsubstituted C₄, C₅, C₆, C₇, C₅, or C₉ alkylene.

39-40. (canceled)

41. The compound of claim 1, wherein R^{2c} is —O—C(═O)R^{2d} or —C(═O)—OR^{2d} and R^{2d} is branched and unsubstituted C₁-C₂₄ alkyl.

42-46. (canceled)

47. The compound of claim 1, wherein G² is —N(C(═O)R^{1d})— and R^{1d} is unbranched and unsubstituted C₃, C₅, or C₉ alkyl.

48. The compound of claim 1, wherein R^{2c} has one of the following structures:

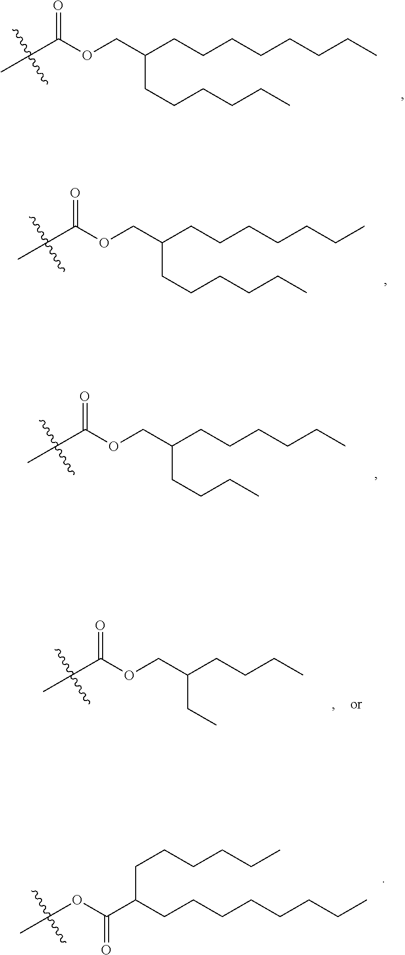 [SMILES: *C(=O)OCC(CCCCCC)CCCCCCCC ; *C(=O)OCC(CCCCCC)CCCCCCC ; *C(=O)OCC(CCCC)CCCCCC ; *C(=O)OCC(CC)CCCC ; *OC(=O)C(CCCCCC)CCCCCCCC]

49. (canceled)

50. The compound of claim 1, wherein R³ is C₅-C₆ cycloalkyl, methyl, ethyl, or n-propyl optionally substituted with —OH, 5-membered heterocyclyl, 5-membered heteroaryl, —N(CH₃)₂, or methoxy.

51-53. (canceled)

54. The compound of claim 1, wherein R³ is —CH₃ or has one of the following structures:

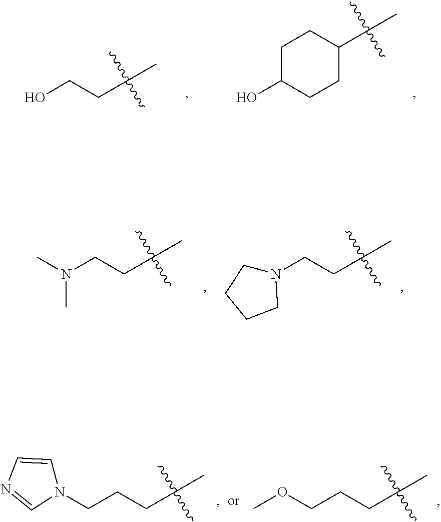

55. The compound of claim 1, wherein L^{1a} is C₂-C₁₂ alkylene.

56. The compound of claim 1, wherein L² is C₂-C₁₂ alkylene.

57-60. (canceled)

61. The compound of claim 1, wherein L^{1a} is unbranched and unsubstituted C₂, C₃, C₄, C₅, C₆, C₇, C₅, or C₉ alkylene.

62. The compound of claim 1, wherein L² is unbranched and unsubstituted C₂, C₃, C₄, C₅, C₆, C₇, C₅, or C₉ alkylene.

63. The compound of claim 1, wherein L^{1a} is unbranched and unsubstituted C₇ alkylene and L² is unbranched and unsubstituted C₂ or C₃ alkylene or L^{1a} and L² are both unbranched and unsubstituted C₂, C₃, C₄, C₅, C₆, C₇, C₅, or C₉ alkylene.

64-68. (canceled)

69. The compound of claim 1, wherein L^{1b} is unbranched and unsubstituted C₁, C₂, C₄, C₅, C₆, C₇, C₅, or C₉ alkylene.

70-73. (canceled)

74. The compound of claim 1, wherein the compound has one of the following structures: 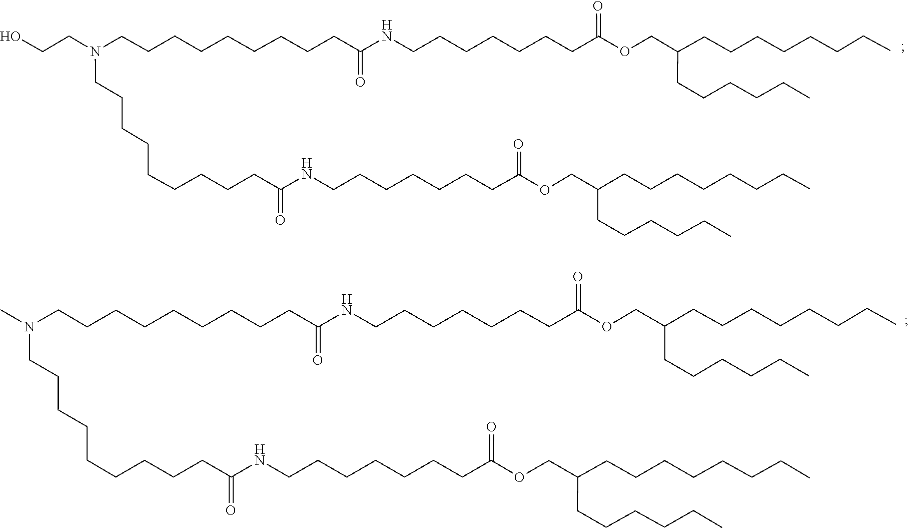 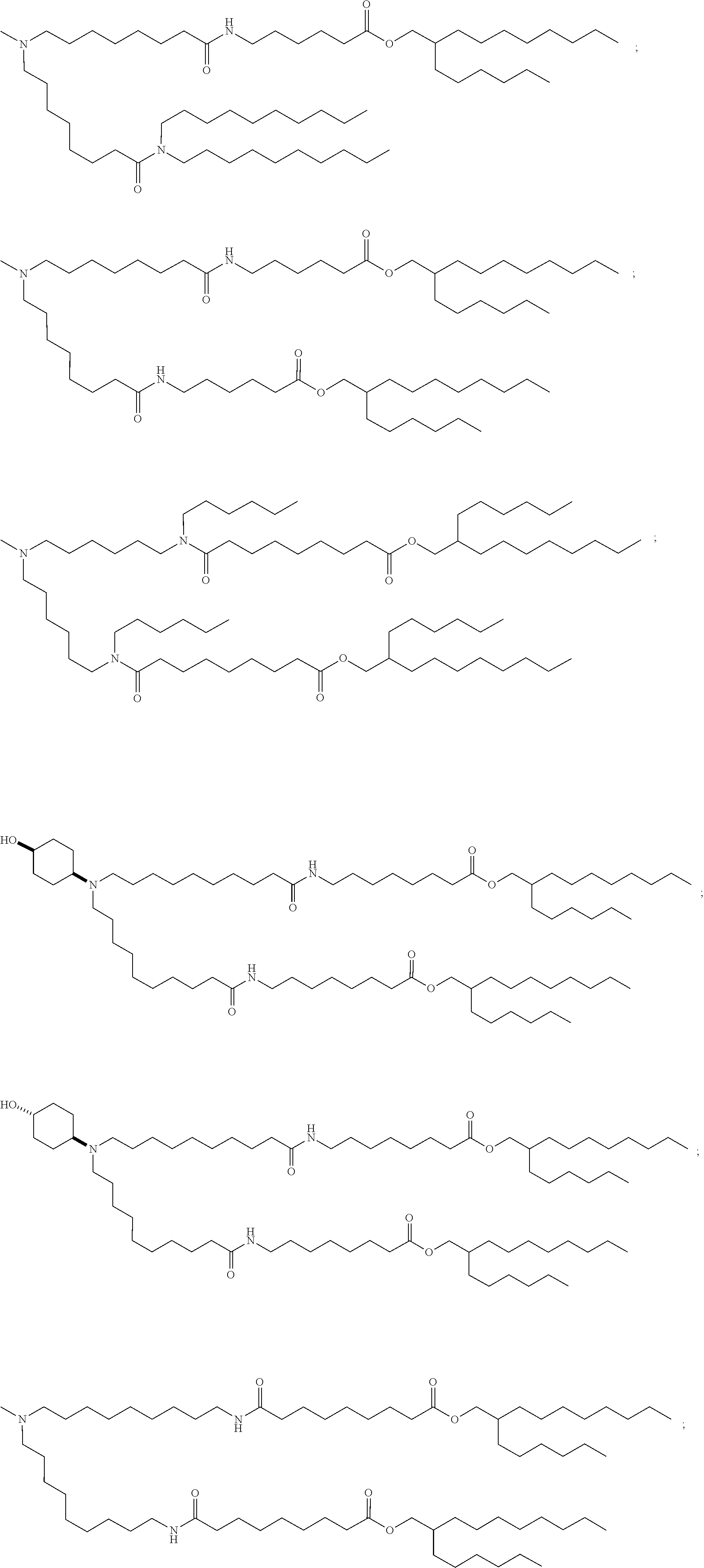 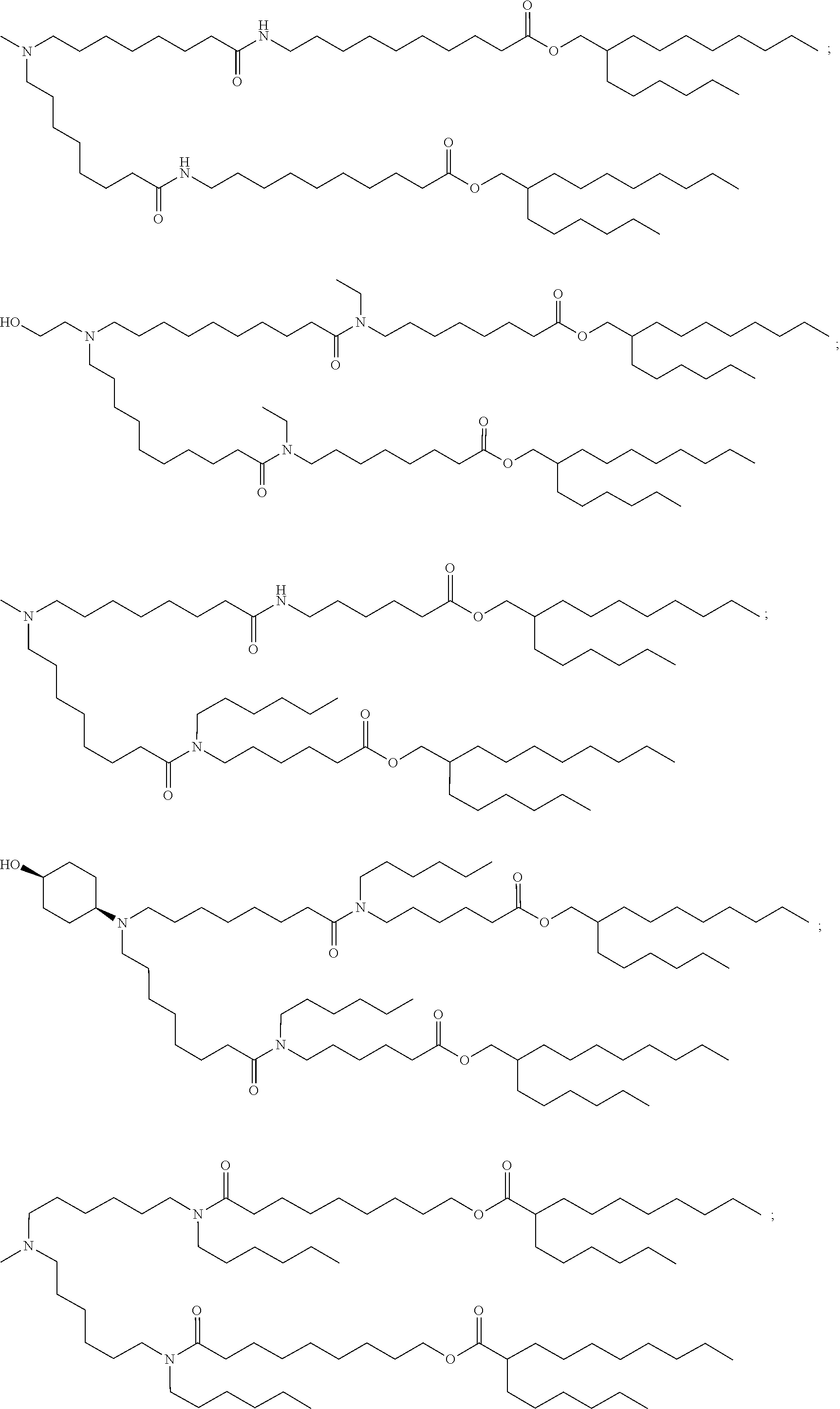 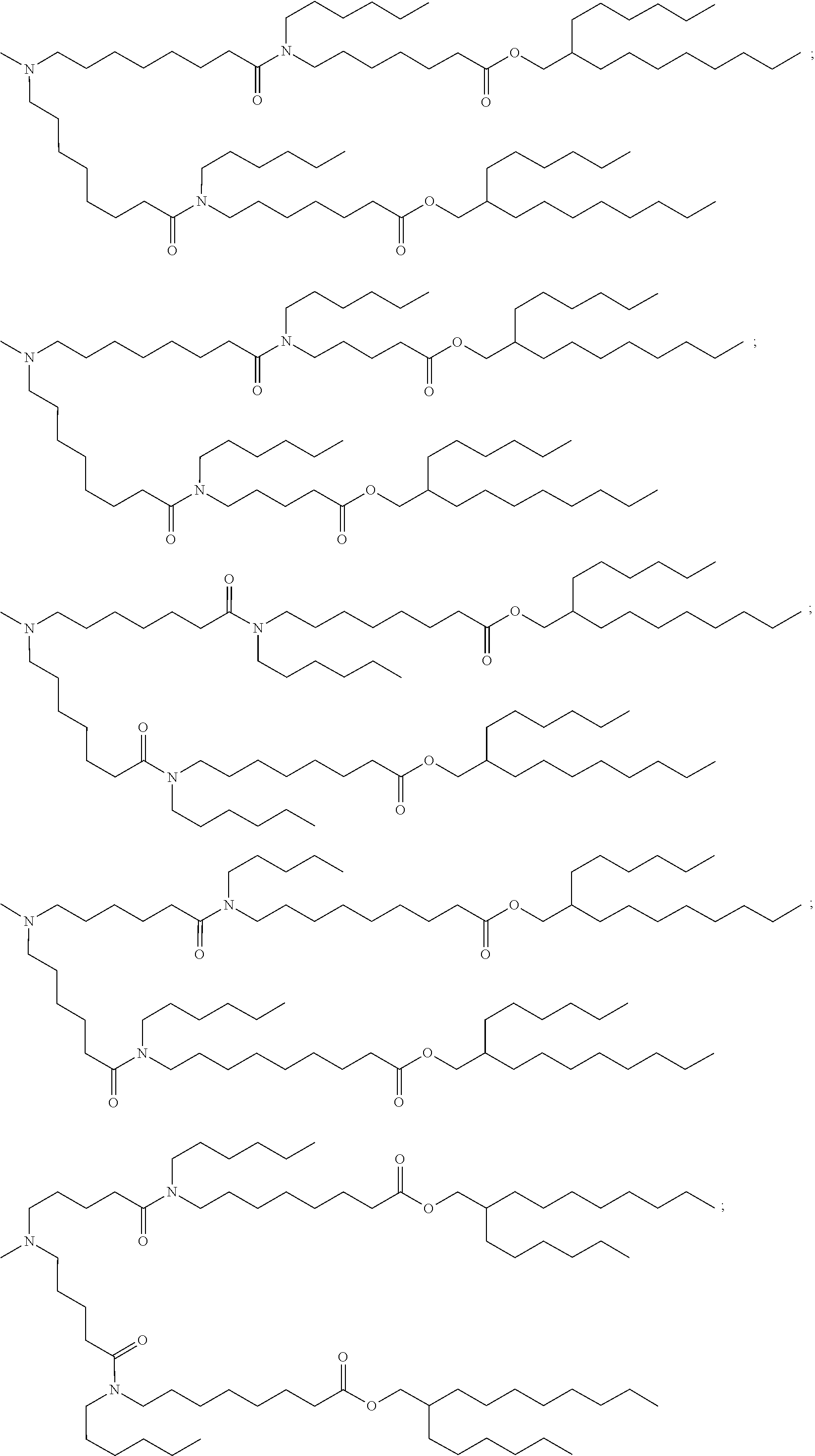 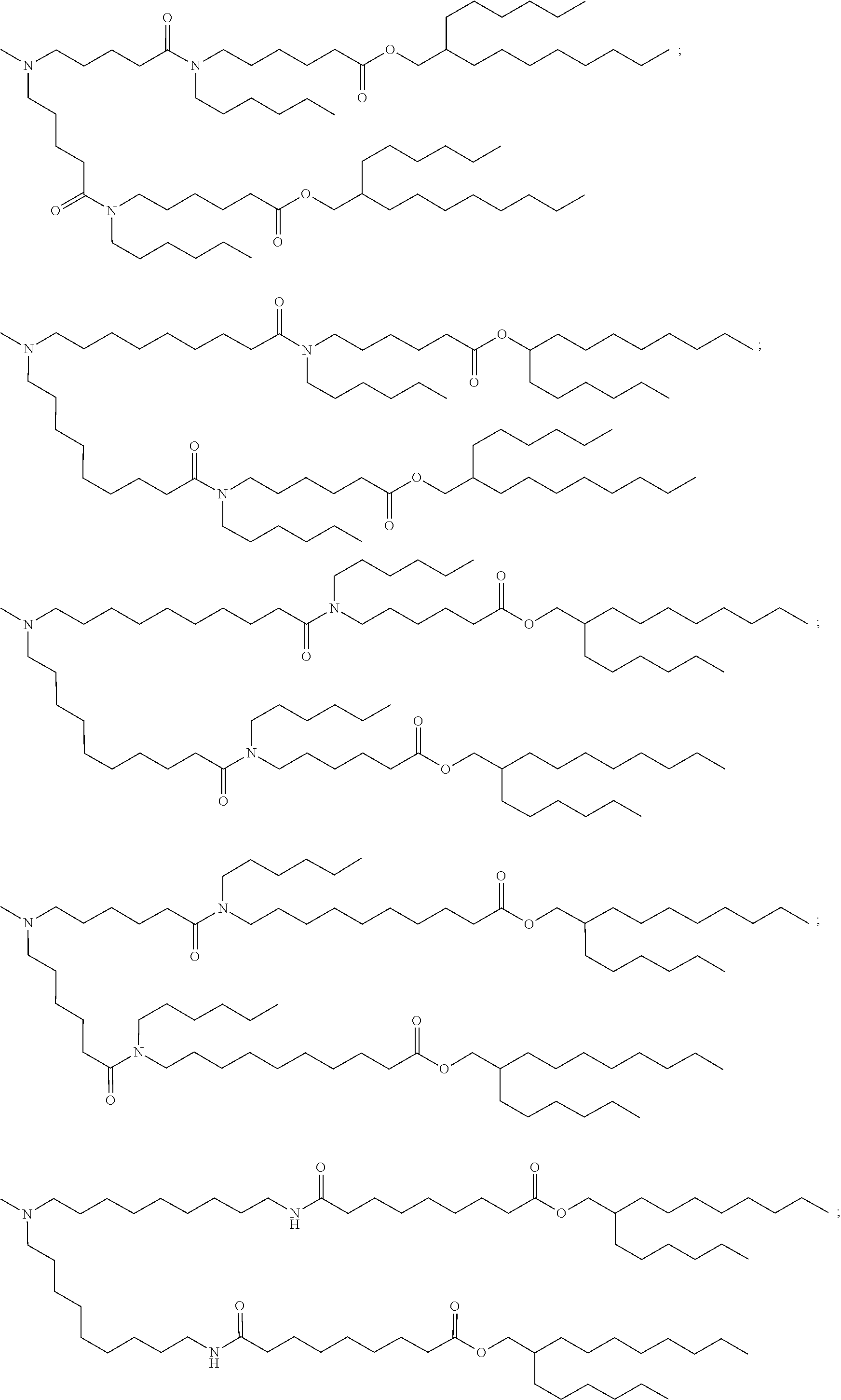 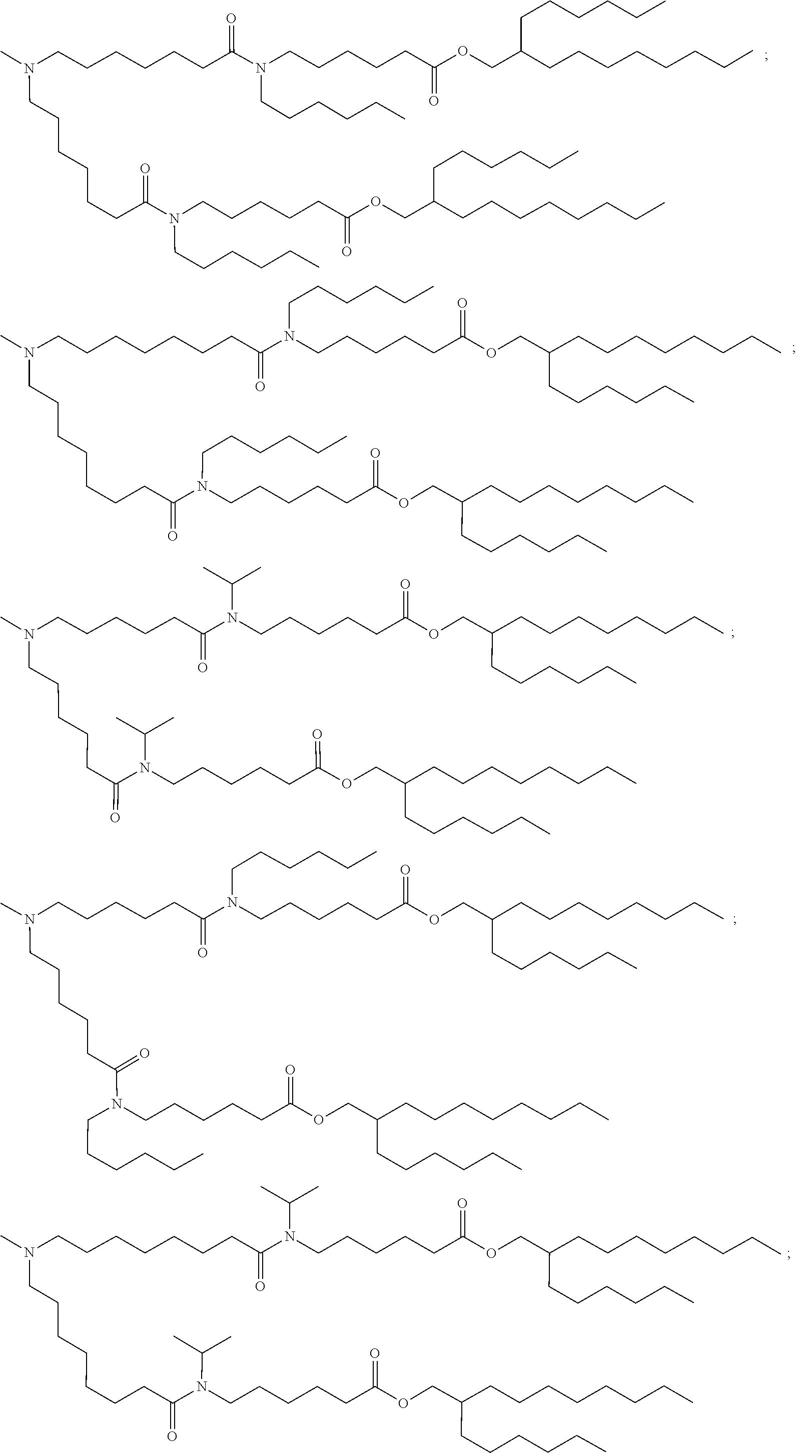 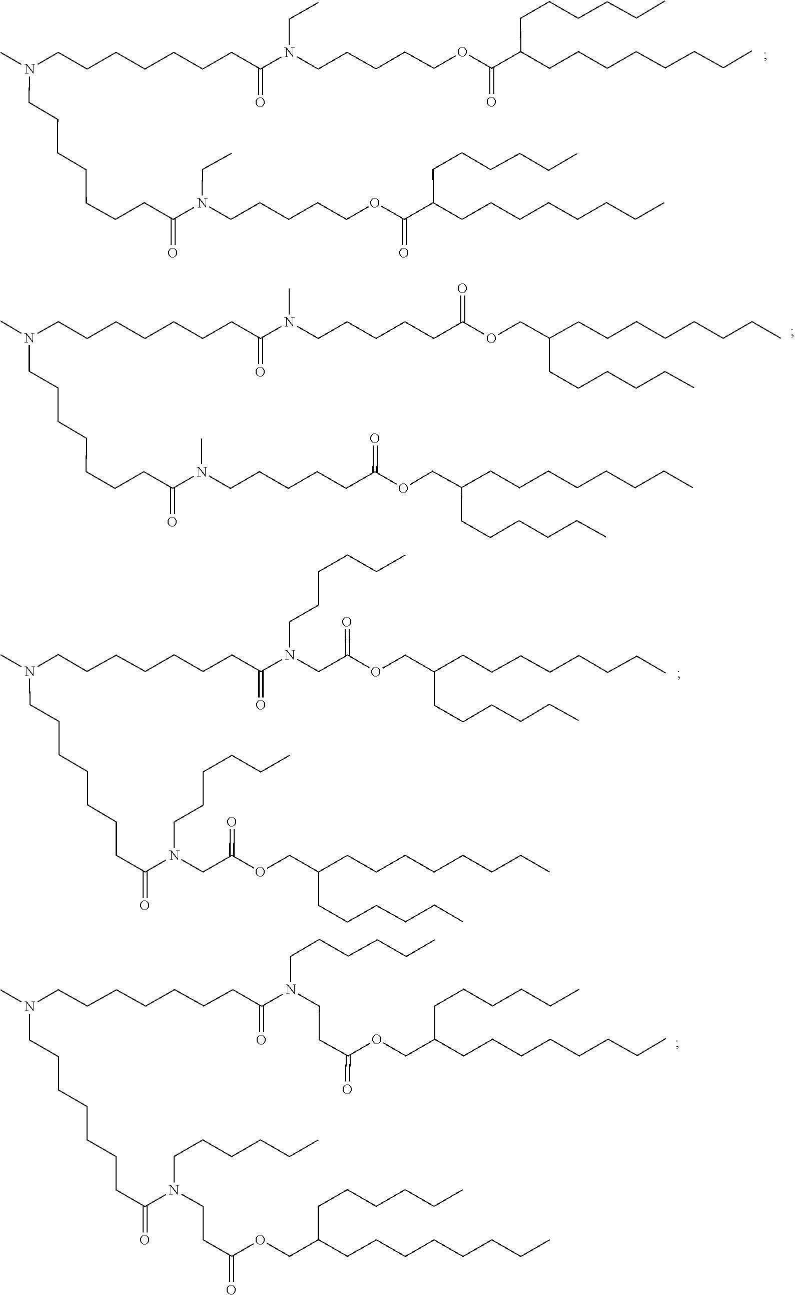 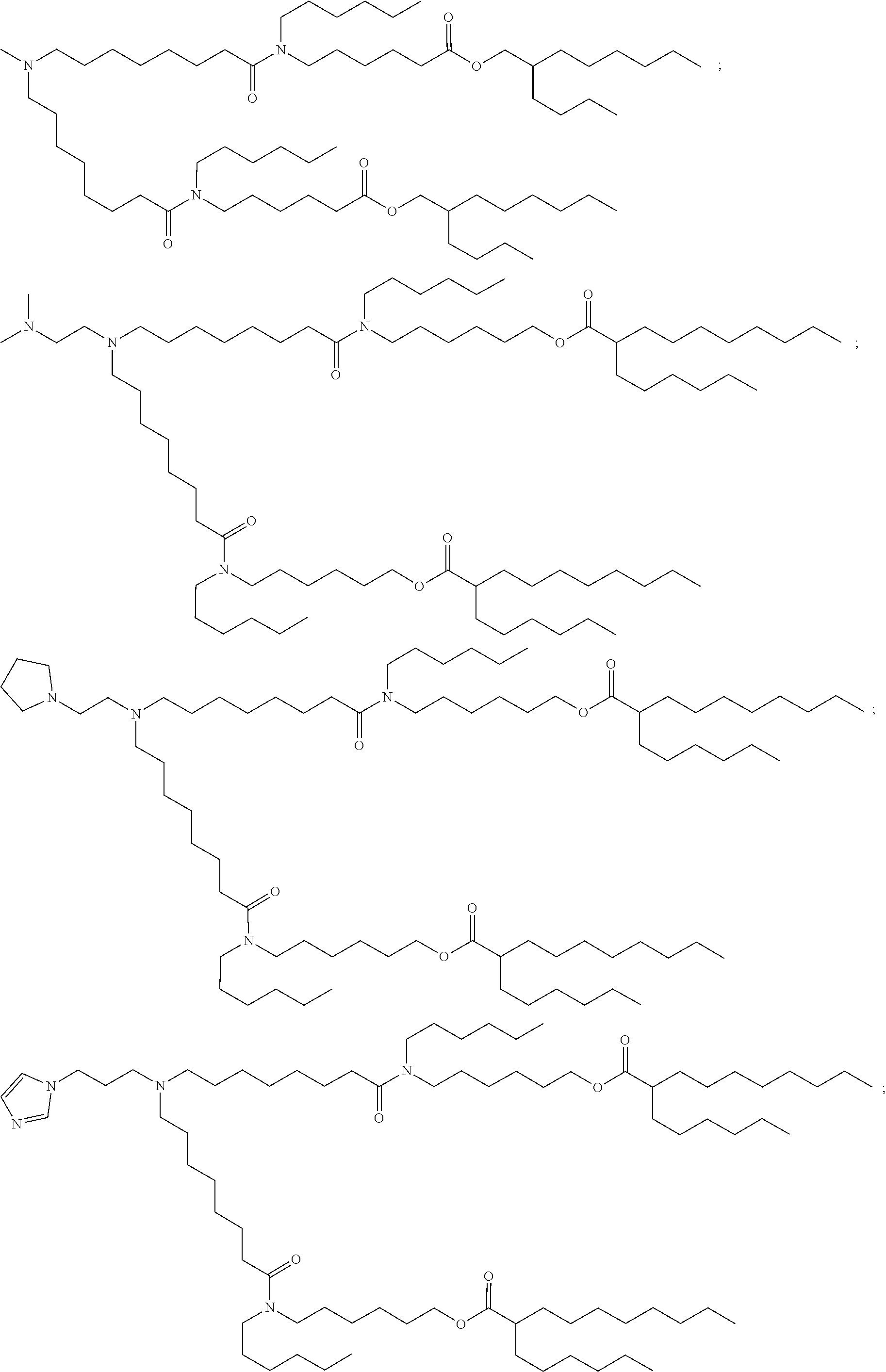 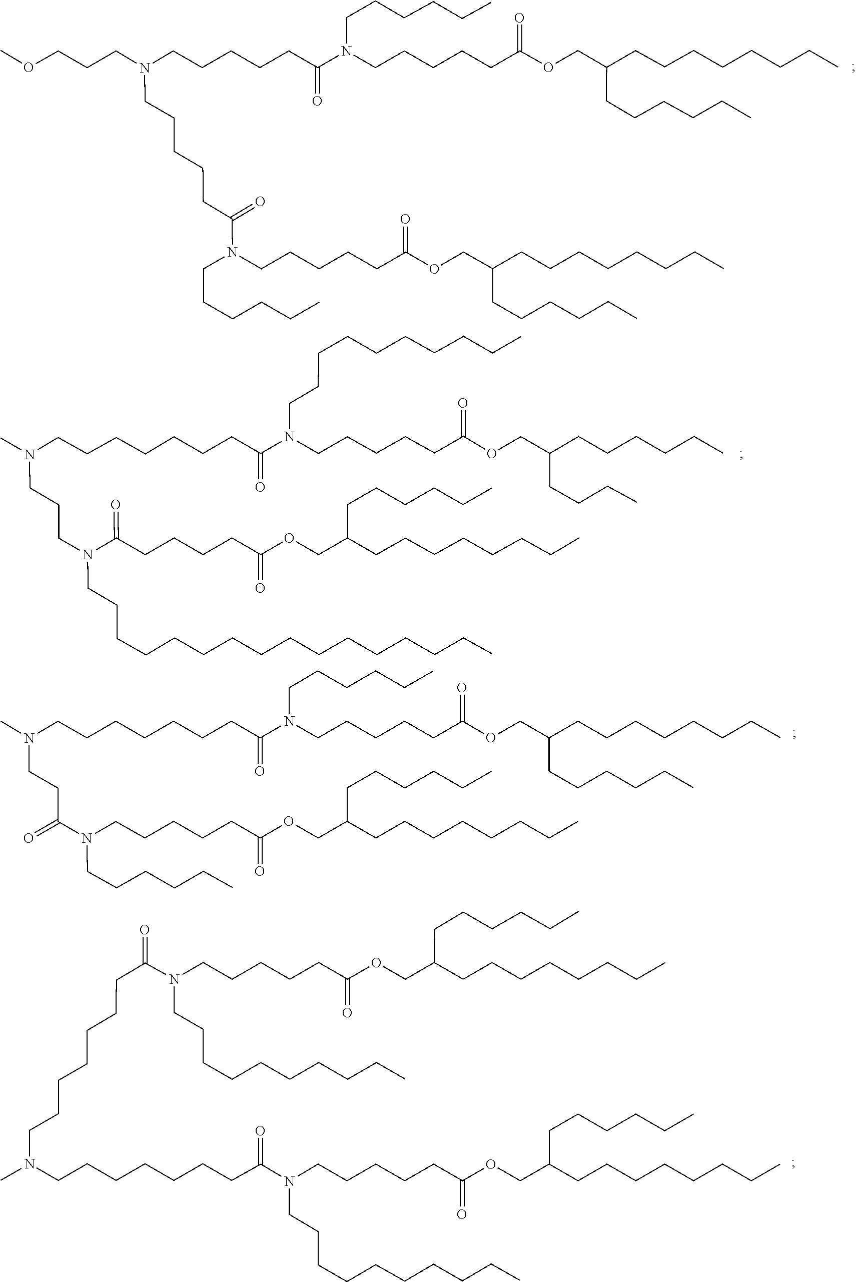 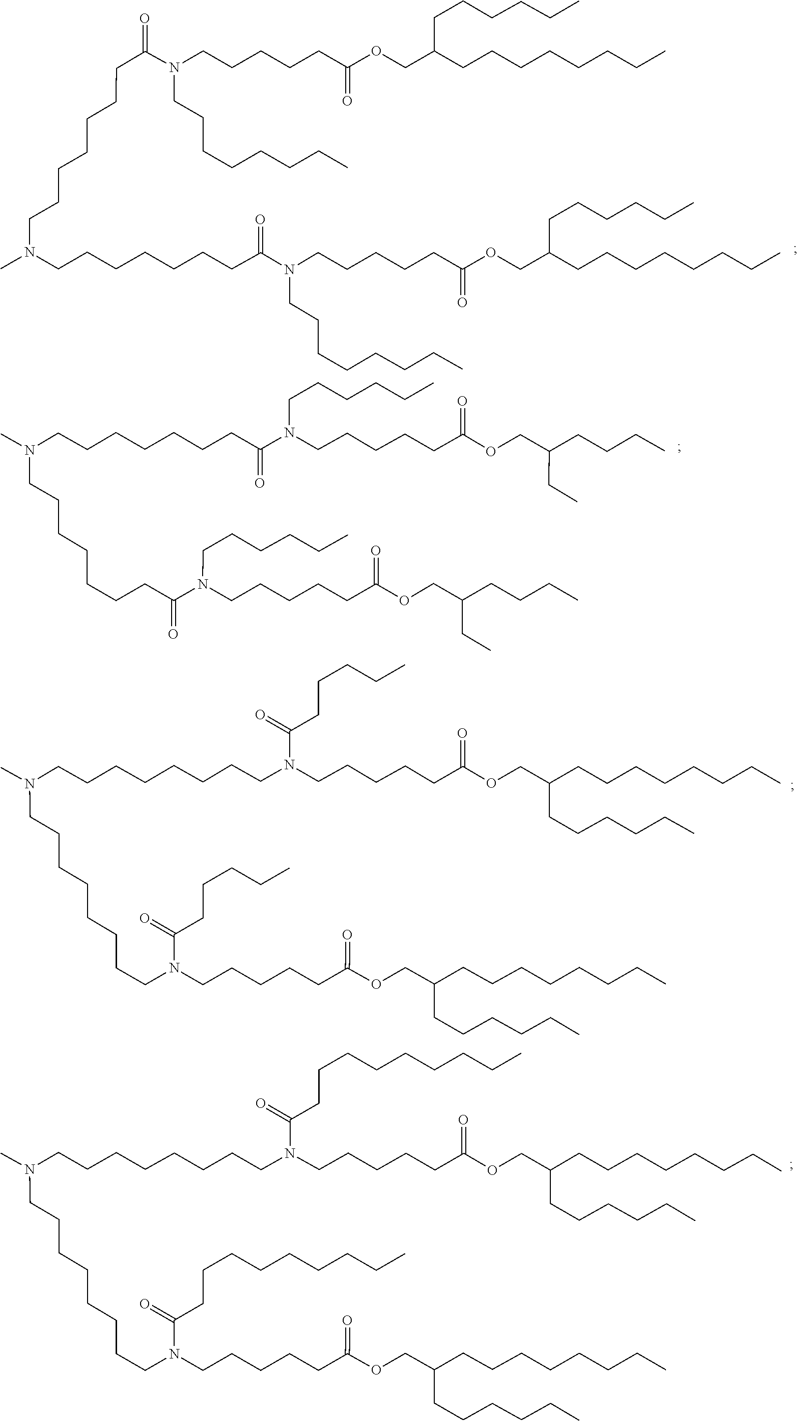 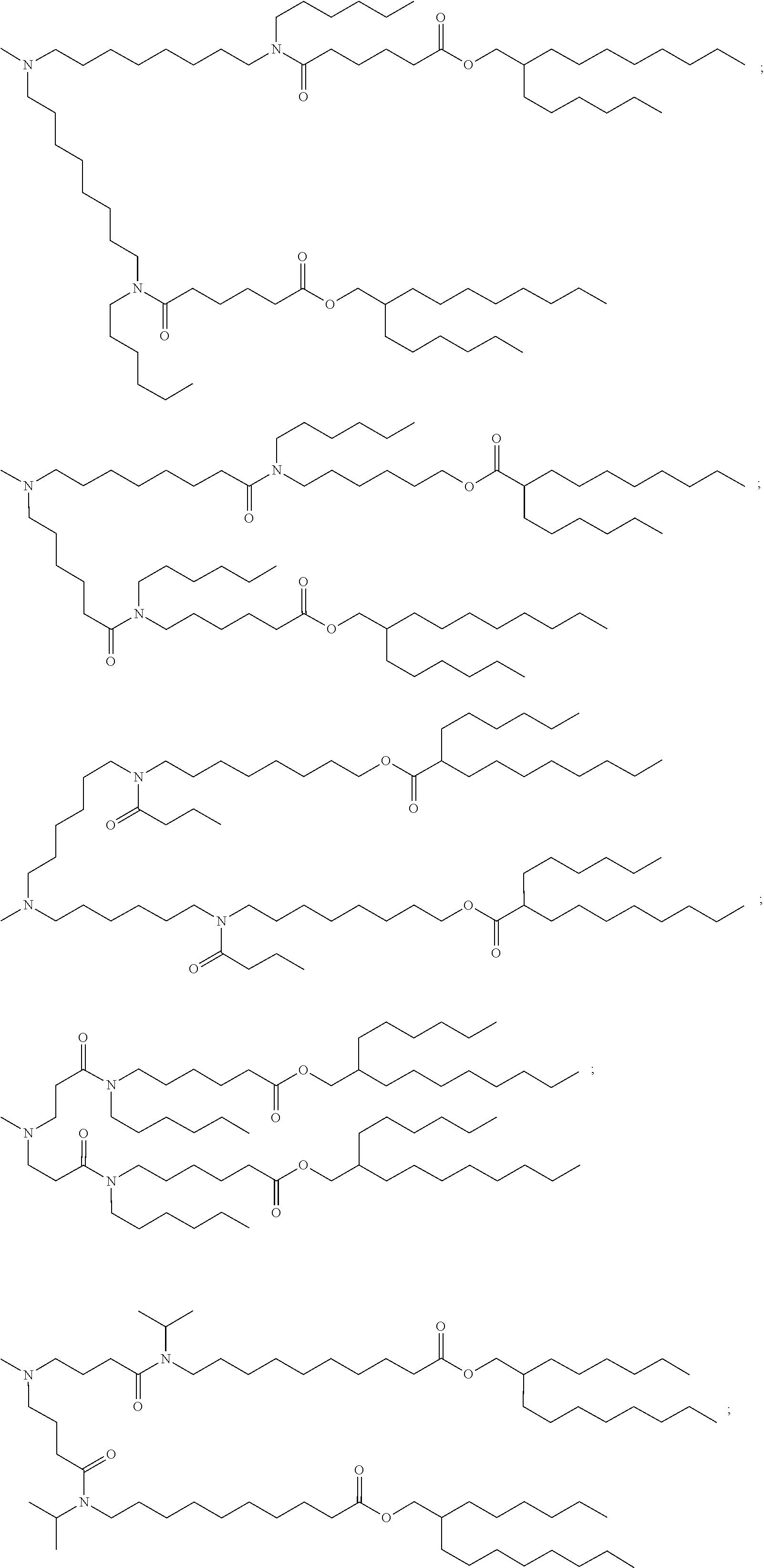 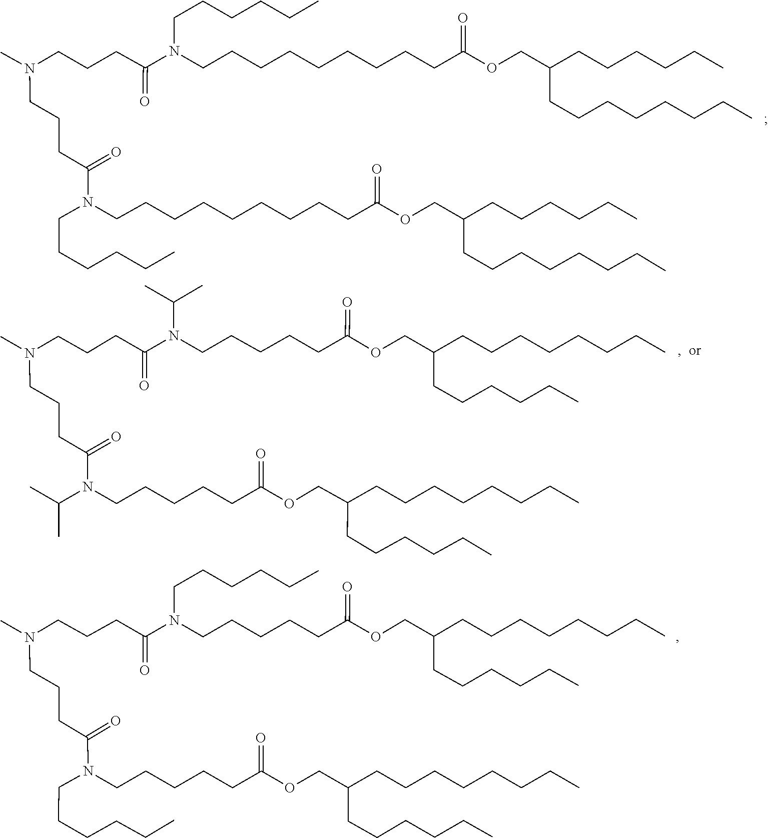
or a pharmaceutically acceptable salt, tautomer, or stereoisomer thereof.

75. A lipid nanoparticle comprising the compound of any claim 1 and a therapeutic agent.

76. A composition comprising the compound of claim 1 and a therapeutic agent.

77-90. (canceled)

91. A method for inducing expression of a desired protein in a subject in need thereof, the method comprising administering a therapeutically effective amount of the lipid nanoparticle of claim 75 to the subject, wherein the therapeutic agent comprises a nucleic acid.

92-95. (canceled)
